(19) United States
(12) Patent Application Publication
(10) Pub. No.: US 2025/0304539 A1
(43) Pub. Date: Oct. 2, 2025

(54) TYPE OF NOVEL LIPID COMPOUND AND USE THEREOF

(71) Applicant: NANJING VAZYME BIOTECH CO., LTD., NANJING (CN)

(72) Inventors: Yan ZHANG, NANJING (CN); Xiaoyu XU, NANJING (CN); Jinbo LI, NANJING (CN); Yi FENG, NANJING (CN); Hao KONG, NANJING (CN)

(21) Appl. No.: 18/864,118

(22) PCT Filed: May 22, 2023
(86) PCT No.: PCT/CN2023/095573

§ 371 Date: Nov. 8, 2024

(30) Foreign Application Priority Data
Jun. 16, 2022 (CN) 202210679273.8

(51) Int. Cl. C07D 249/04; A61K 9/1272; A61K 48/00; C07D 249/06; C07D 405/12

(52) U.S. Cl. CPC C07D 249/04; A61K 9/1272; A61K 48/0033; C07D 249/06; C07D 405/12

(57) ABSTRACT

The present disclosure relates to a type of novel lipid compound and a use thereof, and specifically relates to a type of novel lipid compound and to a lipid nanodelivery carrier comprising the compound; the compound has improved biocompatibility, effectively reduces the toxic side effects of nucleic acid drug lipid nanoparticles, increases the variety of ionizable lipid compounds, and provides more choices for the delivery of nucleic acid drugs.

### CROSS-REFERENCE TO RELATED APPLICATIONS

This application is a U.S. National Stage Application of PCT Application No. PCT/CN2023/095573, filed on May 22, 2023, which claims priority to Chinese Patent Application No. 202210679273.8, filed on Jun. 16, 2022, the entire contents of which are hereby incorporated by reference in their entirety for all purpose.

### TECHNICAL FIELD

The present disclosure belongs to the field of biomedicine and biotechnology, and relates to a novel lipid compound and a system for constructing a lipid nanodelivery carrier by using the lipid compound to deliver active ingredients.

### BACKGROUND

When mRNA drugs are used in clinical treatment, it is necessary to overcome many obstacles in the delivery of exogenous mRNA, and therefore, safe and effective carriers are needed to deliver mRNA to target tissues, organs and cells in the body to play a corresponding role. Lipid nanoparticles (LNPs) are currently the most advanced mRNA delivery system, which are safe and efficient and represents the mainstream of mRNA carrier development in the future.

Lipid nanoparticles (LNPs) are a mature delivery platform for nucleic acids, generally including the nucleic acid that are desired to be delivered, cationic/ionizable/lipoid and some auxiliary lipids, wherein the auxiliary lipids are usually phospholipids, cholesterol and PEGylation lipids. LNPs are currently the most advanced nanodrug delivery system for nucleic acid drugs. How to prepare stable, safe and delivery-efficient LNPs, achieve rapid conversion of gene drugs and achieve targeted delivery to different tissues are key issues in this field, and the solution to these issues depends on a lipid molecule library with diverse structures and functions.

Ionizable lipids (ILs), also called pH-dependent lipids, are almost uncharged and neutral at physiological pH. Under acidic conditions, ILs are positively charged, which facilitates assembly with negatively charged mRNA by electrostatic interactions. ILs remain neutral in the neutral environment of the body fluids. ILs are protonated as the pH decreases below the pKa of the ILs during cellular internalization, and due to the proton sponge effect, LNPs osmotically swell and rupture, releasing mRNA. The chemical structure of ILs plays a decisive role in factors such as the stability, biosafety, and delivery efficiency of LNPs.

The ionizable lipid structure generally comprises three moieties: a hydrophilic head, a hydrophobic tail, and a linker moiety connecting the head and the tail. Based on current research progress and clinical conditions, degradable and multibranched tails are favorable structural properties for the future development of ionizable lipids. For example, the structure of the ionizable lipid SM-102 used by Moderna in the COVID-19 vaccine includes a tertiary amine head, three branches, and a tail containing an ester bond. As the most critical component in LNPs, screening for safer and more efficient ionizable lipids has always been the focus of improving the performance of LNPs.

### SUMMARY

The present disclosure provides a type of novel lipid compound with a simple preparation method, low toxicity and high biocompatibility, which enriches the types of lipid compounds and provides more choices for the delivery of nucleic acid drugs. The lipid compound of the present disclosure, when prepared into LNPs with other lipids, is capable of effectively delivering mRNA or drug molecules into cells to exert biological functions.

The present disclosure provides a compound of formula (I), or a salt or an isomer thereof, wherein the formula (I) has a structure as shown below:

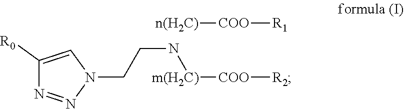

wherein R₀ is selected from C₁₋₄ alkyl, C₃₋₆ cycloalkyl, aryl or heteroaryl, the C₁₋₄ alkyl or C₃₋₆ cycloalkyl is optionally substituted with one or more —OH, —NR₀ₐR_{0b}, —NHR₀ₐ, —OR₀ₐ or 4-7 membered heterocyclyl containing 1-2 N, O or S atoms, and the aryl or heteroaryl is optionally substituted with C₁₋₃ alkyl, C₁₋₃ alkylalkoxy or halogen;
R₀ₐ and Rob are each independently selected from C₁₋₃ alkyl;
R₁ and R₂ are independently selected from C₂₋₂₀ alkyl or C₄₋₁₈ alkenyl; and
n and m are each independently selected from an integer of 1-9.

In some embodiments, R₀ in the compound of formula (I) is selected from —CH₂CH₃, —CH₂CH₂CH₃, —CH₂(CH₃)₂, —CH₂CH(CH₃)₂, —(CH₂)₃CH₃, —C(CH₃)₃, —CH(CH₃)CH₂CH₃, —CH₂CH₂OH, —CH(OH)CH₃, —CH₂CH₂CH₂OH, —CH₂CH(CH₃)OH, —CH(CH₃)CH₂OH, —C(OH)(CH₃)₂, —CH(OH)CH₂CH₃, —CH₂N(CH₂CH₃)₂, —CH₂N(CH₃)₂, —CH₂NHCH₃, —CH₂NHCH₂CH₃, —CH₂N(CH₃)CH₂CH₃, —CH(OCH₂CH₃)₂,

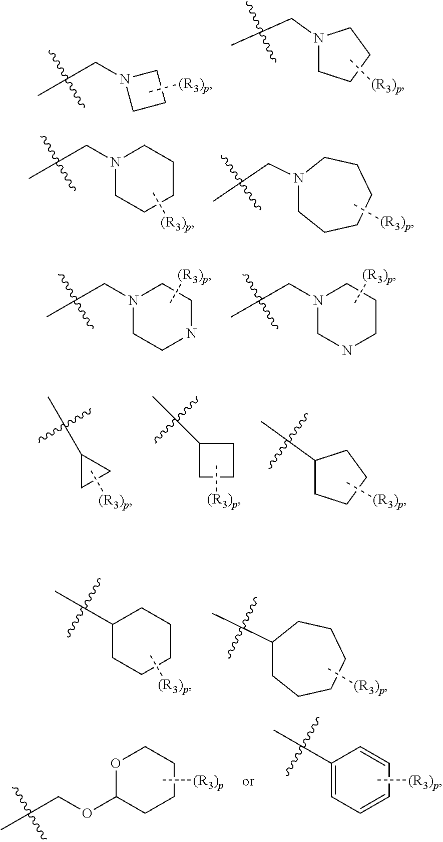

wherein R₃ is selected from C₁₋₃ alkyl, C₁₋₃ alkoxy or halogen, and p is selected from a natural number of 0-2.

In some embodiments, R₀ in the compound of formula (I) is selected from —CH₂CH₂CH₃, —CH₂CH(CH₃)₂, —CH₂CH₂OH, —CH₂CH₂CH₂OH, —CH₂CH(CH₃)OH, —CH(OH)CH₂CH₃, —CH₂N(CH₂CH₃)₂, —CH₂NHCH₃, —CH₂N(CH₃)₂, —CH(OCH₂CH₃)₂,

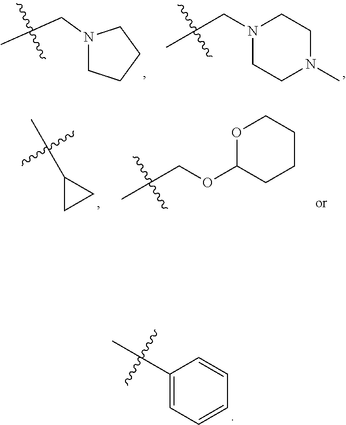

In some embodiments, R₀ in the compound of formula (I) is selected from —CH₂CH₂OH, —CH₂CH₂CH₂OH, —CH₂CH(CH₃)OH, —CH(OH)CH₂CH₃, —CH₂N(CH₂CH₃)₂, —CH₂N(CH₃)₂,

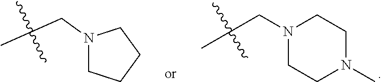

In some embodiments, R₀ in the compound of formula (I) is selected from —CH₂N(CH₂CH₃)₂, —CH₂N(CH₃)₂,

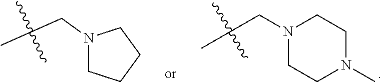

In some embodiments, R₁ in the compound of formula (I) is selected from C₈₋₂₀ alkyl or C₈₋₁₈ alkenyl.

In some embodiments, R₂ in the compound of formula (I) is selected from C₈₋₂₀ alkyl or C₈₋₁₈ alkenyl.

In some embodiments, R₁ in the compound of formula (I) is selected from —(CH₂)₇CH₃, (CH₂)₅CH₃, —(CH₂)₉CH₃, —(CH₂)₁₀CH₃, —(CH₂)₁₁CH₃, —(CH₂)₁₂CH₃, —CH((CH₂)₄CH₃)₂, —CH((CH₂)₅CH₃)₂, —CH((CH₂)₆CH₃)₂, —CH((CH₂)₇CH₃)₂, —CH((CH₂)₈CH₃)₂, (CH₂)₂CH═CH(CH₂)₄CH₃, —(CH₂)₃CH═CH(CH₂)₃CH₃, —(CH₂)₄CH═CH(CH₂)₂CH₃, (CH₂)₂CH═CH(CH₂)₃CH₃, —(CH₂)₃CH═CH(CH₂)₂CH₃, —(CH₂)₂CH═CH(CH₂)₅CH₃, (CH₂)₄CH═CH(CH₂)₃CH₃, —(CH₂)₅CH═CH(CH₂)₂CH₃, —(CH₂)₃CH═CH(CH₂)₄CH₃, (CH₂)₄CH═CH(CH₂)₄CH₃, —(CH₂)₃CH═CH(CH₂)₅CH₃, —(CH₂)₅CH═CH(CH₂)₃CH₃, (CH₂)₂CH═CH(CH₂)₆CH₃, —(CH₂)₆CH═CH(CH₂)₂CH₃, —(CH₂)₄CH═CH(CH₂)₅CH₃, (CH₂)₅CH═CH(CH₂)₄CH₃, —(CH₂)₃CH═CH(CH₂)₆CH₃, —(CH₂)₆CH═CH(CH₂)₃CH₃, (CH₂)₂CH═CH(CH₂)₈CH₃ or —(CH₂)₈CH═CH(CH₂)₂CH₃.

In some embodiments, R₁ in the compound of formula (I) is selected from —(CH₂)₇CH₃, (CH₂)₈CH₃, —(CH₂)₉CH₃, —(CH₂)₁₀CH₃, —(CH₂)₁₁CH₃, —(CH₂)₁₂CH₃, —CH((CH₂)₄CH₃)₂, —CH((CH₂)₅CH₃)₂, —CH((CH₂)₆CH₃)₂, —CH((CH₂)₇CH₃)₂, —CH((CH₂)₈CH₃)₂.

In some embodiments, R₂ in the compound of formula (I) is selected from —(CH₂)₇CH₃, (CH₂)₈CH₃, —(CH₂)₉CH₃, —(CH₂)₁₀CH₃, —(CH₂)₁₁CH₃, —(CH₂)₁₂CH₃, —CH((CH₂)₄CH₃)₂, —CH((CH₂)₆CH₃)₂, —CH((CH₂)₇CH₃)₂, —CH((CH₂)₈CH₃)₂, (CH₂)₃CH═CH(CH₂)₃CH₃, —(CH₂)₄CH═CH(CH₂)₂CH₃, —(CH₂)₂CH═CH(CH₂)₄CH₃, (CH₂)₂CH═CH(CH₂)₅CH₃, —(CH₂)₂CH═CH(CH₂)₃CH₃, —(CH₂)₃CH═CH(CH₂)₂CH₃, (CH₂)₃CH═CH(CH₂)₄CH₃, —(CH₂)₄CH═CH(CH₂)₃CH₃, —(CH₂)₅CH═CH(CH₂)₂CH₃, (CH₂)₅CH═CH(CH₂)₃CH₃, —(CH₂)₄CH═CH(CH₂)₄CH₃, —(CH₂)₃CH═CH(CH₂)₅CH₃, (CH₂)₆CH═CH(CH₂)₂CH₃, —(CH₂)₄CH═CH(CH₂)₅CH₃, —(CH₂)₂CH═CH(CH₂)₆CH₃, (CH₂)₃CH═CH(CH₂)₆CH₃, —(CH₂)₆CH═CH(CH₂)₃CH₃, —(CH₂)₅CH═CH(CH₂)₄CH₃, —CH((CH₂)₅CH₃)₂, (CH₂)₂CH═CH(CH₂)₈CH₃ or —(CH₂)₈CH═CH(CH₂)₂CH₃.

In some embodiments, R₂ in the compound of formula (I) is selected from —(CH₂)₇CH₃, (CH₂)₈CH₃, —(CH₂)₉CH₃, —(CH₂)₁₀CH₃, —(CH₂)₁₁CH₃, —(CH₂)₁₂CH₃, —CH((CH₂)₄CH₃)₂, —CH((CH₂)₅CH₃)₂, —CH((CH₂)₆CH₃)₂, —CH((CH₂)₇CH₃)₂, —CH((CH₂)₈CH₃)₂.

In some embodiments, R₁ and R₂ in the compound of formula (I) are independently selected from —(CH₂)₇CH₃, —(CH₂)₈CH₃, —(CH₂)₉CH₃, —(CH₂)₁₀CH₃, —(CH₂)₁₁CH₃, —(CH₂)₁₂CH₃, —CH((CH₂)₄CH₃)₂, —CH((CH₂)₅CH₃)₂, —CH((CH₂)₆CH₃)₂, —CH((CH₂)₇CH₃)₂, —CH((CH₂)₈CH₃)₂,
(CH₂)₂CH═CH(CH₂)₄CH₃, —(CH₂)₃CH═CH(CH₂)₃CH₃, —(CH₂)₄CH═CH(CH₂)₂CH₃,
(CH₂)₂CH═CH(CH₂)₃CH₃, —(CH₂)₃CH═CH(CH₂)₂CH₃, —(CH₂)₂CH═CH(CH₂)₅CH₃,
(CH₂)₄CH═CH(CH₂)₃CH₃, —(CH₂)₅CH═CH(CH₂)₂CH₃, —(CH₂)₃CH═CH(CH₂)₄CH₃,
(CH₂)₅CH═CH(CH₂)₃CH₃, —(CH₂)₄CH═CH(CH₂)₄CH₃, —(CH₂)₃CH═CH(CH₂)₅CH₃,
(CH₂)₂CH═CH(CH₂)₆CH₃, —(CH₂)₆CH═CH(CH₂)₂CH₃, —(CH₂)₄CH═CH(CH₂)₅CH₃,
(CH₂)₃CH═CH(CH₂)₆CH₃, —(CH₂)₆CH═CH(CH₂)₃CH₃, —(CH₂)₅CH═CH(CH₂)₄CH₃,
(CH₂)₂CH═CH(CH₂)₈CH₃ or —(CH₂)₈CH═CH(CH₂)₂CH₃.

In some embodiments, n and m in the compound of formula (I) are each independently selected from an integer of 3-9. In some embodiments, n and m in the compound of formula (I) are each independently selected from an integer of 4-8. In some embodiments, n and m in the compound of formula (I) are each independently selected from an integer of 5-7. In some embodiments, n and m in the compound of formula (I) are each independently selected from an integer of 5, 6 or 7.

In some embodiments, in the compound of formula (I), n is 5, m is 7, R₁ is —(CH₂)₁₀CH₃, and R₂ is —CH((CH₂)₈CH₃)₂.

In some embodiments, in the compound of formula (I), both n and m are 7, and both R₁ and R₂ are —CH((CH₂)₈CH₃)₂.

In some embodiments, in the compound of formula (I), n is 5, m is 7, R₁ is (CH₂)₃CH═CH(CH₂)₅CH₃, and R₂ is —CH((CH₂)₈CH₃)₂.

In some embodiments, the compound of formula (I), or the salt or the isomer thereof is selected from the following compounds LipidA-1 to LipidA-14, LipidB22-1 to LipidB22-15, LipidB23-1 to LipidB23-15, or salts or isomers thereof.

The present disclosure further provides a delivery carrier, comprising the compound of the present disclosure and an accessory molecule. In some embodiments, the accessory molecule comprises: a phospholipid, a structural lipid and a PEGylation lipid.

In some embodiments, in the delivery carrier of the present disclosure, the molar ratio of the compound of the present disclosure to the accessory molecule is 1:1.

In some embodiments, in the delivery carrier of the present disclosure, the content of the compound of the present disclosure is 20%-80%, the content of the PEGylation lipid compound is 1%-10%, the content of the structural lipid is 10%-50%, and the content of the phospholipid is 5%-30%, by mole percent. Optionally, the content of the compound of formula (I) is selected from 20%, 25%, 30%, 35%, 40%, 45%, 50%, 55%, 60%, 65%, 70%, 75% or 80% by mole percent. Optionally, the content of the compound of formula (I) is selected from 45%, 46%, 47%, 48%, 49%, 50%, 51%, 52%, 53%, 54% or 55% by mole percent. Optionally, the content of the PEGylation lipid compound is selected from 1%, 1.5%, 2%, 2.5%, 3%, 3.5%, 4%, 4.5%, 5%, 5.5%, 6%, 6.5%, 7%, 7.5%, 8%, 8.5%, 9%, 9.5% or 10% by mole percent. Optionally, the content of the PEGylation lipid compound is selected from 1%, 1.1%, 1.2%, 1.3%, 1.4%, 1.5%, 1.6%, 1.7%, 1.8%, 1.9% or 2% by mole percent. Optionally, the content of the structural lipid is 10%, 15%, 20%, 25%, 30%, 35%, 40%, 45% or 50% by mole percent. Optionally, the content of the structural lipid is 35%, 35.5%, 36%, 36.5%, 37%, 37.5%, 38%, 38.5%, 39%, 39.5% or 40% by mole percent. Optionally, the content of the phospholipid is selected from 5%, 10%, 15%, 20%, 25% or 30% by mole percent. Optionally, the content of the phospholipid is 5%, 6%, 7%, 8%, 9%, 10%, 11%, 12%, 13%, 14% or 15% by mole percent.

In some embodiments, the phospholipid is selected from at least one of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine (DOPE), 1,2-dioleoyl-sn-glycero-3-phosphoethanolamine (ME 16.0PE), 1,2-diphytanoyl-sn-glycero-3-phosphoethanolamine 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), dipalmitoyl phosphatidylglycerol (DPPG), palmitoyl oleoyl phosphatidylethanolamine (POPE), distearoyl-phosphatidylethanolamine (DSPE), dipalmitoyl phosphatidylethanolamine (DPPE), dimyristoyl phosphatidylethanolamine (DMPE), 1-stearoyl-2-oleoyl-stearoylethanolamine (SOPE), 1-stearoyl-2-oleoyl-phosphatidylcholine (SOPC), sphingomyelin, phosphatidylcholine, phosphatidylethanolamine, phosphatidylserine, phosphatidylinositol, phosphatidic acid, palmitoyloleoylphosphatidylcholine, lysophosphatidylcholine or lysophosphatidylethanolamine (LPE).

In some embodiments, the structural lipid is selected from at least one of cholesterol, coprosterol, sitosterol, ergosterol, campesterol, stigmasterol, brassicasterol, tomatine, ursolic acid or α-tocopherol.

In some embodiments, the PEGylation lipid compound is selected from at least one of PEG-modified phosphatidylethanolamine, PEG-modified phosphatidic acid, PEG-modified ceramide, PEG-modified dialkylamine, PEG-modified diacylglycerol, PEG-modified dialkylglycerol, and the above PEG-modified lipids modified with cell-targeting ligands.

In some embodiments, the delivery carrier further comprises an active ingredient, and the active ingredient is selected from at least one of DNA, RNA, a protein or a pharmaceutically active molecule.

In some embodiments, the delivery carrier is a lipid nanoparticle.

In some embodiments, the lipid nanoparticle further comprises an active ingredient, and the active ingredient is selected from at least one of DNA, RNA, a protein or a pharmaceutically active molecule.

In some embodiments, the RNA is selected from at least one of mRNA, siRNA, aiRNA, miRNA, dsRNA, aRNA, lncRNA, an antisense nucleotide (ASO) or an oligonucleotide.

In some embodiments, the protein is selected from at least one of an antibody, an enzyme, a recombinant protein, a polypeptide or a short peptide.

The present disclosure further provides a method for preparing lipid nanoparticles, the method comprising step (1) of mixing and dissolving the compound of the present disclosure, a PEGylation lipid, a structural lipid and a phospholipid in an absolute ethanol solution.

Optionally, the method further comprises step (2) of mixing the solution of the step (1) with an active ingredient to form lipid nanoparticles.

Optionally, the compound of the present disclosure, the PEGylation lipid, the structural lipid and the phospholipid are dissolved and mixed in ethanol, and then mixed with an active ingredient to form lipid nanoparticles.

In one embodiment, the present disclosure further provides the use of the compound of the present disclosure in the preparation of lipid nanoparticles.

In some embodiments, the compound of the present disclosure is selected from the following compounds, or salts or isomers thereof:

| Name | Structure |
|---|---|
| LipidA-1 | 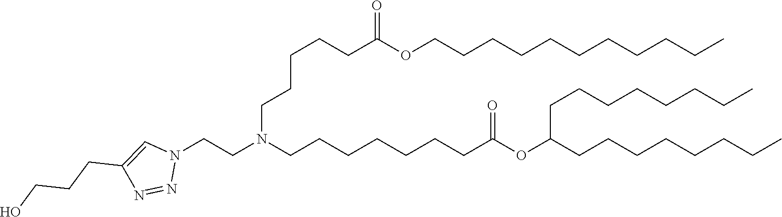 [SMILES: CCCCCCCCCCCOC(=O)CCCCCN(CCCCCCCC(=O)OC(CCCCCCCC)CCCCCCCC)CCn1cc(CCCO)nn1] |
| LipidA-2 | 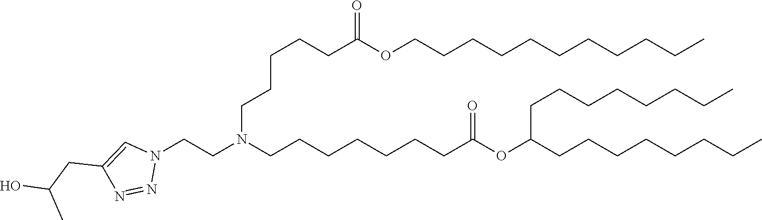 [SMILES: CCCCCCCCCCCOC(=O)CCCCCN(CCCCCCCC(=O)OC(CCCCCCCC)CCCCCCCC)CCn1cc(CC(C)O)nn1] |
| LipidA-3 | 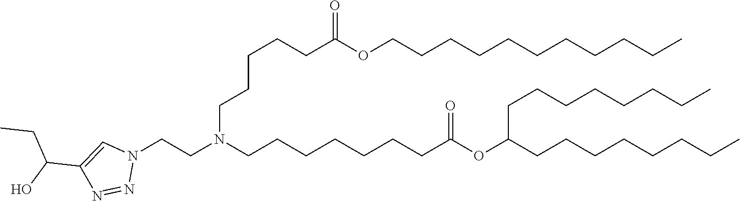 [SMILES: CCCCCCCCCCCOC(=O)CCCCCN(CCCCCCCC(=O)OC(CCCCCCCC)CCCCCCCC)CCn1cc(C(O)CC)nn1] |
| LipidA-4 | 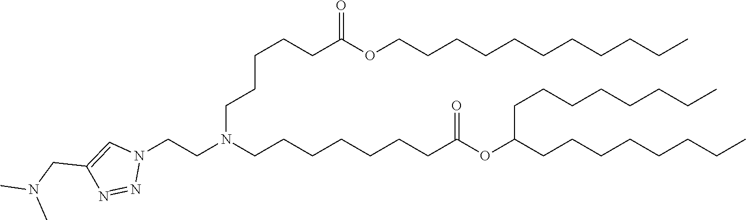 [SMILES: CCCCCCCCCCCOC(=O)CCCCCN(CCCCCCCC(=O)OC(CCCCCCCC)CCCCCCCC)CCn1cc(CN(C)C)nn1] |
| LipidA-5 | 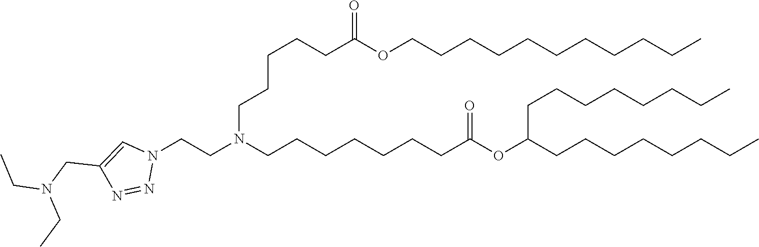 [SMILES: CCCCCCCCCCCOC(=O)CCCCCN(CCCCCCCC(=O)OC(CCCCCCCC)CCCCCCCC)CCn1cc(CN(CC)CC)nn1] |
| LipidA-6 | 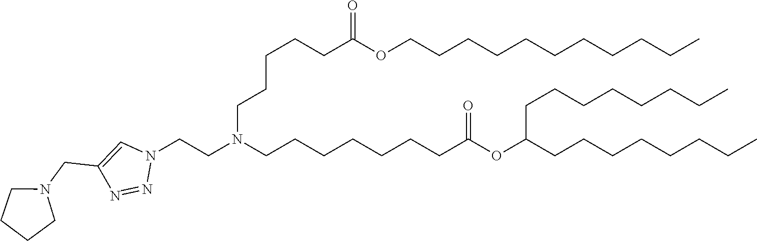 [SMILES: CCCCCCCCCCCOC(=O)CCCCCN(CCCCCCCC(=O)OC(CCCCCCCC)CCCCCCCC)CCn1cc(CN2CCCC2)nn1] |
| LipidA-7 | 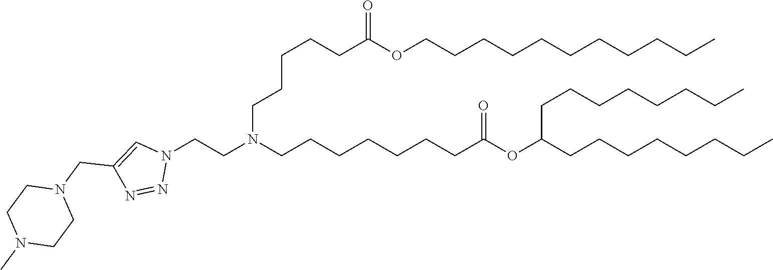 [SMILES: CCCCCCCCCCCOC(=O)CCCCCN(CCCCCCCC(=O)OC(CCCCCCCC)CCCCCCCC)CCn1cc(CN2CCN(C)CC2)nn1] |
| LipidA-8 | 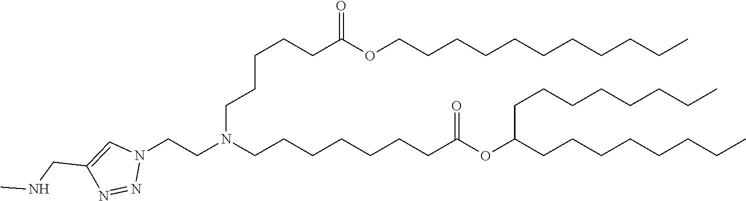 [SMILES: CCCCCCCCCCCOC(=O)CCCCCN(CCCCCCCC(=O)OC(CCCCCCCC)CCCCCCCC)CCn1cc(CNC)nn1] |
| LipidA-9 | 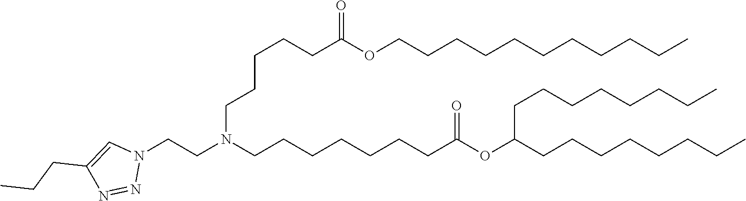 [SMILES: CCCCCCCCCCCOC(=O)CCCCCN(CCCCCCCC(=O)OC(CCCCCCCC)CCCCCCCC)CCn1cc(CCC)nn1] |
| LipidA-10 | 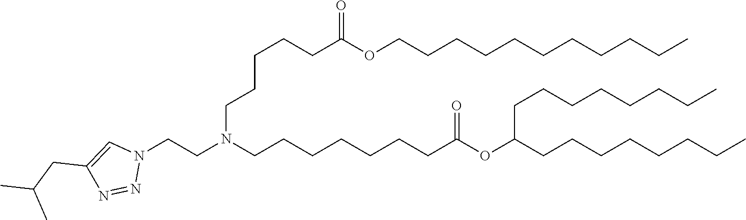 [SMILES: CCCCCCCCCCCOC(=O)CCCCCN(CCCCCCCC(=O)OC(CCCCCCCC)CCCCCCCC)CCn1cc(CC(C)C)nn1] |
| LipidA-11 | 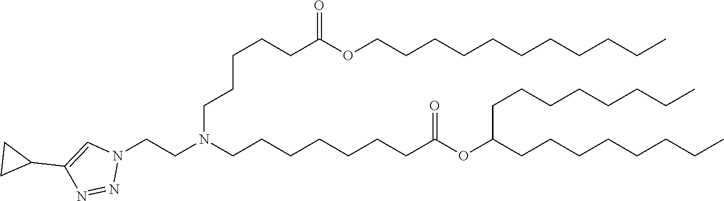 [SMILES: CCCCCCCCCCCOC(=O)CCCCCN(CCCCCCCC(=O)OC(CCCCCCCC)CCCCCCCC)CCn1cc(C2CC2)nn1] |
| LipidA-12 | 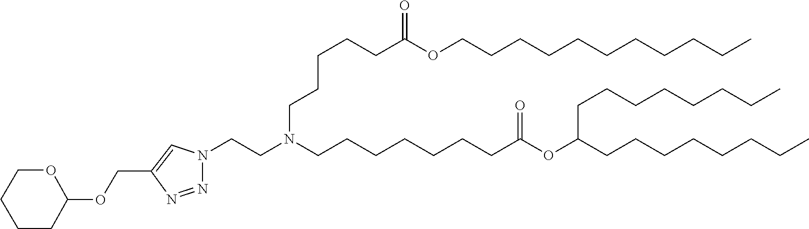 [SMILES: CCCCCCCCCCCOC(=O)CCCCCN(CCCCCCCC(=O)OC(CCCCCCCC)CCCCCCCC)CCn1cc(COC2CCCCO2)nn1] |
| LipidA-13 | 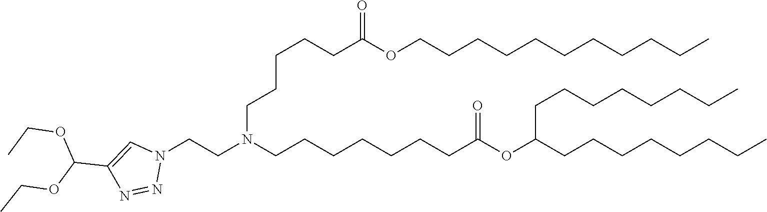 [SMILES: CCCCCCCCCCCOC(=O)CCCCCN(CCCCCCCC(=O)OC(CCCCCCCC)CCCCCCCC)CCn1cc(C(OCC)OCC)nn1] |
| LipidA-14 | 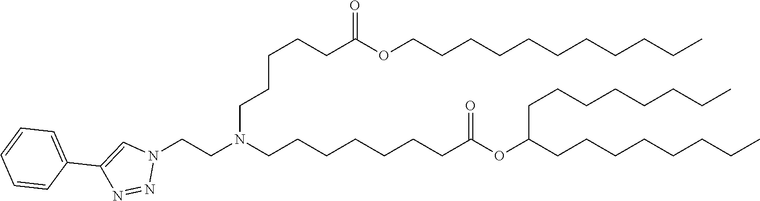 [SMILES: CCCCCCCCCCCOC(=O)CCCCCN(CCCCCCCC(=O)OC(CCCCCCCC)CCCCCCCC)CCn1cc(-c2ccccc2)nn1] |
| LipidB22-1 | 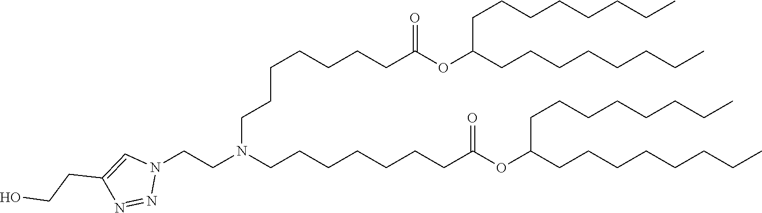 [SMILES: CCCCCCCCC(CCCCCCCC)OC(=O)CCCCCCCN(CCCCCCCC(=O)OC(CCCCCCCC)CCCCCCCC)CCn1cc(CCO)nn1] |
| LipidB22-2 | 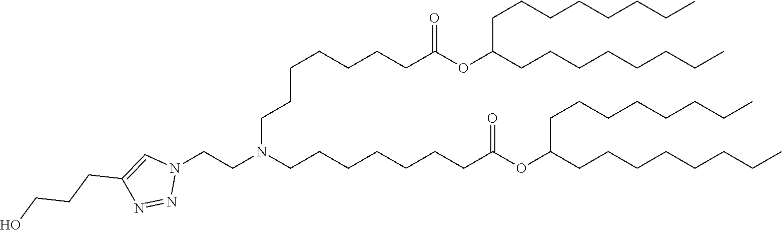 [SMILES: CCCCCCCCC(CCCCCCCC)OC(=O)CCCCCCCN(CCCCCCCC(=O)OC(CCCCCCCC)CCCCCCCC)CCn1cc(CCCO)nn1] |
| LipidB22-3 | 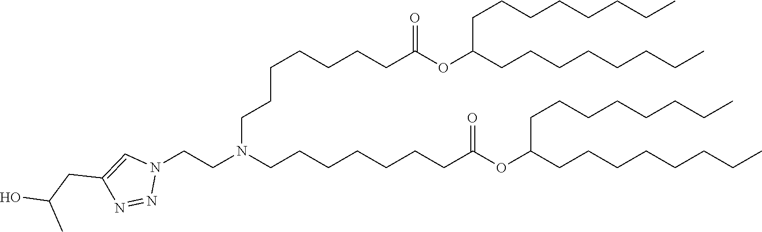 [SMILES: CCCCCCCCC(CCCCCCCC)OC(=O)CCCCCCCN(CCCCCCCC(=O)OC(CCCCCCCC)CCCCCCCC)CCn1cc(CC(C)O)nn1] |
| LipidB22-4 | 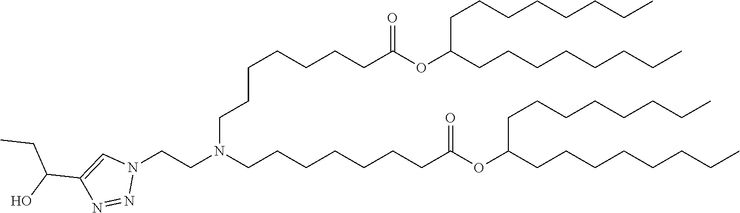 [SMILES: CCCCCCCCC(CCCCCCCC)OC(=O)CCCCCCCN(CCCCCCCC(=O)OC(CCCCCCCC)CCCCCCCC)CCn1cc(C(O)CC)nn1] |
| LipidB22-5 | 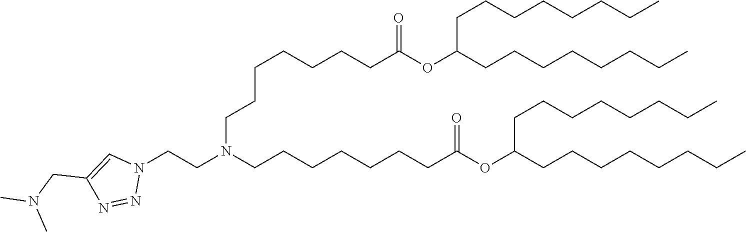 [SMILES: CCCCCCCCC(CCCCCCCC)OC(=O)CCCCCCCN(CCCCCCCC(=O)OC(CCCCCCCC)CCCCCCCC)CCn1cc(CN(C)C)nn1] |
| LipidB22-6 | 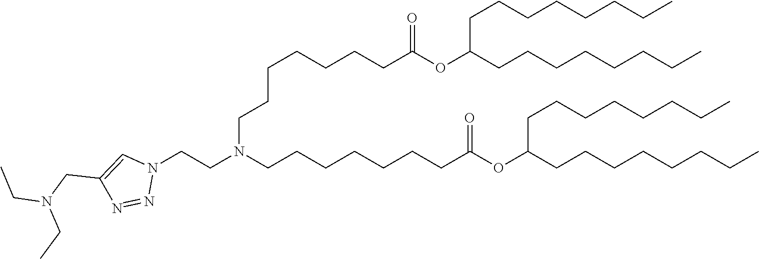 [SMILES: CCCCCCCCC(CCCCCCCC)OC(=O)CCCCCCCN(CCCCCCCC(=O)OC(CCCCCCCC)CCCCCCCC)CCn1cc(CN(CC)CC)nn1] |
| LipidB22-7 | 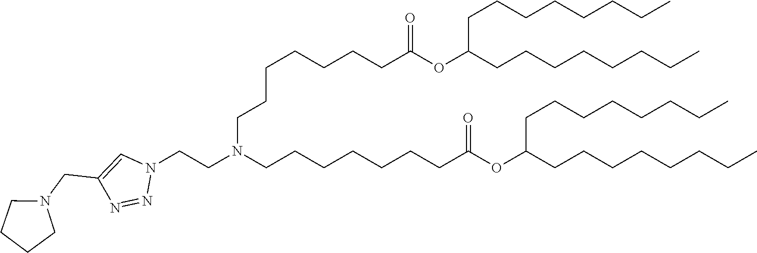 [SMILES: CCCCCCCCC(CCCCCCCC)OC(=O)CCCCCCCN(CCCCCCCC(=O)OC(CCCCCCCC)CCCCCCCC)CCn1cc(CN2CCCC2)nn1] |
| LipidB22-8 | 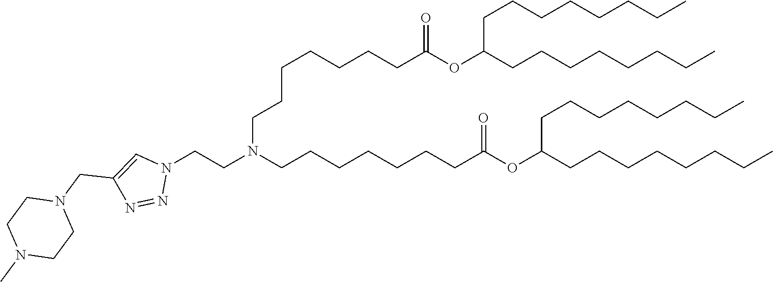 [SMILES: CCCCCCCCC(CCCCCCCC)OC(=O)CCCCCCCN(CCCCCCCC(=O)OC(CCCCCCCC)CCCCCCCC)CCn1cc(CN2CCN(C)CC2)nn1] |
| LipidB22-9 | 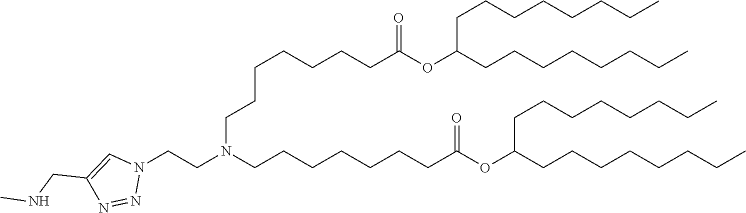 [SMILES: CCCCCCCCC(CCCCCCCC)OC(=O)CCCCCCCN(CCCCCCCC(=O)OC(CCCCCCCC)CCCCCCCC)CCn1cc(CNC)nn1] |
| LipidB22-10 | 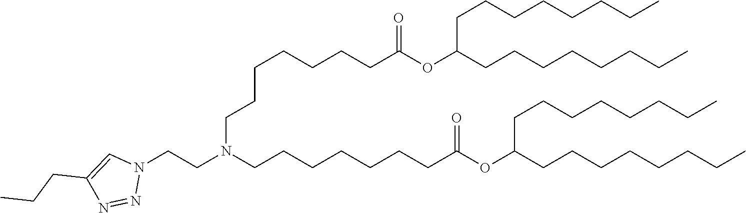 [SMILES: CCCCCCCCC(CCCCCCCC)OC(=O)CCCCCCCN(CCCCCCCC(=O)OC(CCCCCCCC)CCCCCCCC)CCn1cc(CCC)nn1] |
| LipidB22-11 | 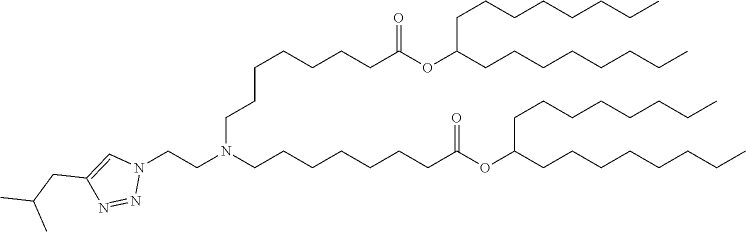 [SMILES: CCCCCCCCC(CCCCCCCC)OC(=O)CCCCCCCN(CCCCCCCC(=O)OC(CCCCCCCC)CCCCCCCC)CCn1cc(CC(C)C)nn1] |
| LipidB22-12 | 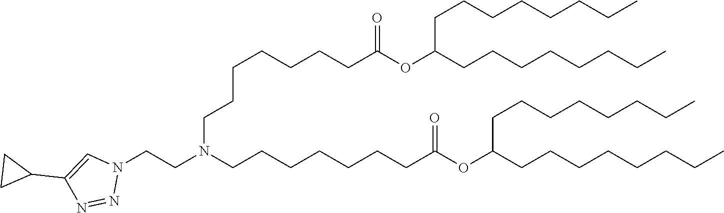 [SMILES: CCCCCCCCC(CCCCCCCC)OC(=O)CCCCCCCN(CCCCCCCC(=O)OC(CCCCCCCC)CCCCCCCC)CCn1cc(C2CC2)nn1] |
| LipidB22-13 | 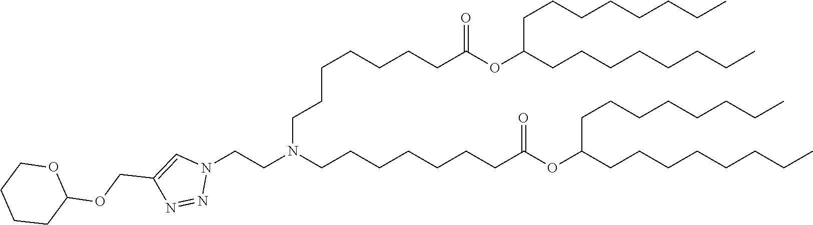 [SMILES: CCCCCCCCC(CCCCCCCC)OC(=O)CCCCCCCN(CCCCCCCC(=O)OC(CCCCCCCC)CCCCCCCC)CCn1cc(COC2CCCCO2)nn1] |
| LipidB22-14 | 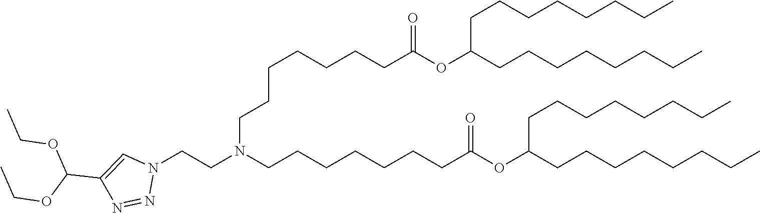 [SMILES: CCCCCCCCC(CCCCCCCC)OC(=O)CCCCCCCN(CCCCCCCC(=O)OC(CCCCCCCC)CCCCCCCC)CCn1cc(C(OCC)OCC)nn1] |
| LipidB22-15 | 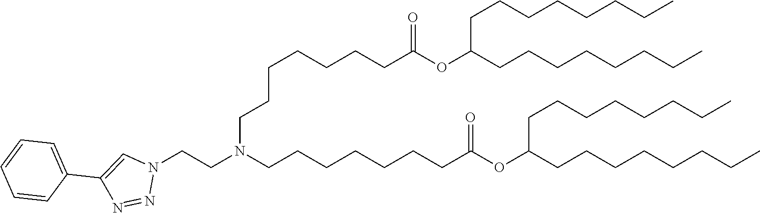 [SMILES: CCCCCCCCC(CCCCCCCC)OC(=O)CCCCCCCN(CCCCCCCC(=O)OC(CCCCCCCC)CCCCCCCC)CCn1cc(-c2ccccc2)nn1] |
| LipidB23-1 | 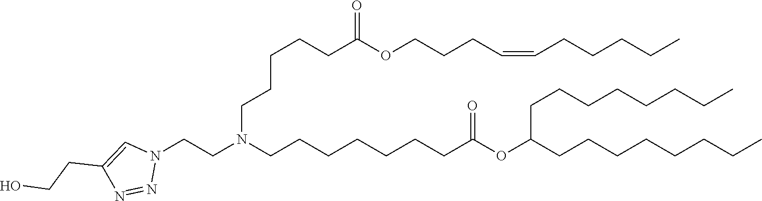 [SMILES: CCCCC/C=C\CCCOC(=O)CCCCCN(CCCCCCCC(=O)OC(CCCCCCCC)CCCCCCCC)CCn1cc(CCO)nn1] |
| LipidB23-2 | 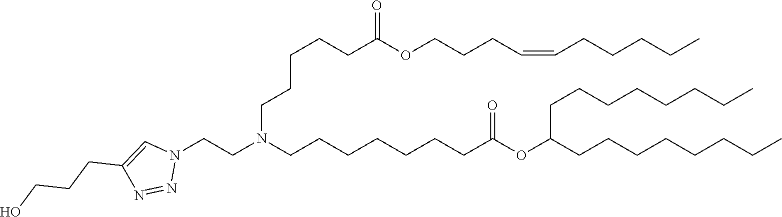 [SMILES: CCCCC/C=C\CCCOC(=O)CCCCCN(CCCCCCCC(=O)OC(CCCCCCCC)CCCCCCCC)CCn1cc(CCCO)nn1] |
| LipidB23-3 | 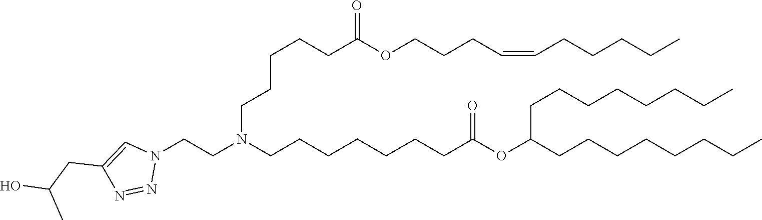 [SMILES: CCCCC/C=C\CCCOC(=O)CCCCCN(CCCCCCCC(=O)OC(CCCCCCCC)CCCCCCCC)CCn1cc(CC(C)O)nn1] |
| LipidB23-4 | 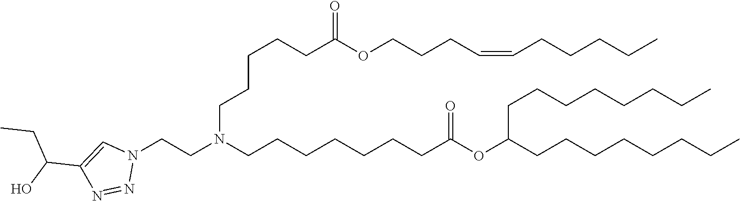 [SMILES: CCCCC/C=C\CCCOC(=O)CCCCCN(CCCCCCCC(=O)OC(CCCCCCCC)CCCCCCCC)CCn1cc(C(O)CC)nn1] |
| LipidB23-5 | 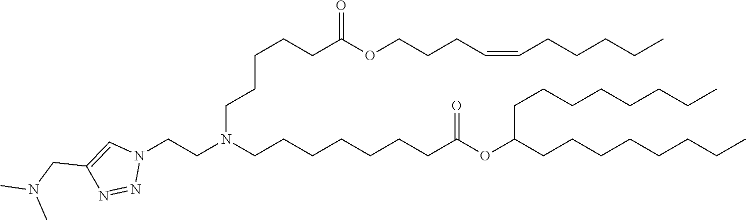 [SMILES: CCCCC/C=C\CCCOC(=O)CCCCCN(CCCCCCCC(=O)OC(CCCCCCCC)CCCCCCCC)CCn1cc(CN(C)C)nn1] |
| LipidB23-6 | 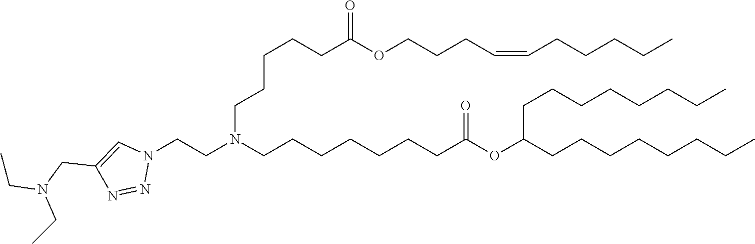 [SMILES: CCCCC/C=C\CCCOC(=O)CCCCCN(CCCCCCCC(=O)OC(CCCCCCCC)CCCCCCCC)CCn1cc(CN(CC)CC)nn1] |
| LipidB23-7 | 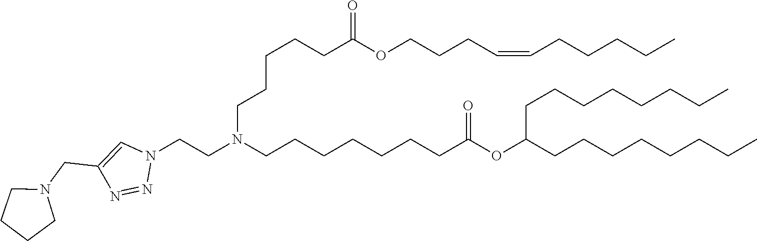 [SMILES: CCCCC/C=C\CCCOC(=O)CCCCCN(CCCCCCCC(=O)OC(CCCCCCCC)CCCCCCCC)CCn1cc(CN2CCCC2)nn1] |
| LipidB23-8 | 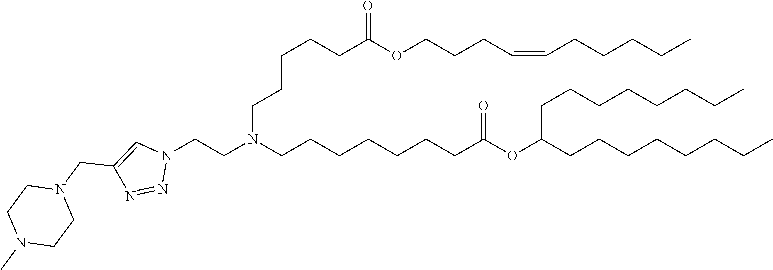 [SMILES: CCCCC/C=C\CCCOC(=O)CCCCCN(CCCCCCCC(=O)OC(CCCCCCCC)CCCCCCCC)CCn1cc(CN2CCN(C)CC2)nn1] |
| LipidB23-9 | 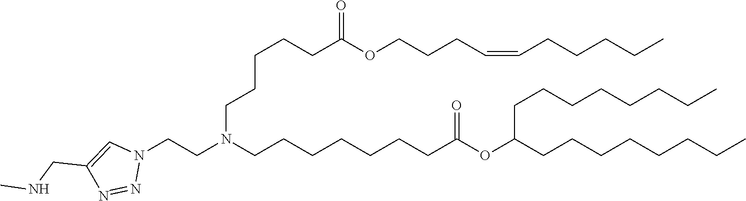 [SMILES: CCCCC/C=C\CCCOC(=O)CCCCCN(CCCCCCCC(=O)OC(CCCCCCCC)CCCCCCCC)CCn1cc(CNC)nn1] |
| LipidB23-10 | 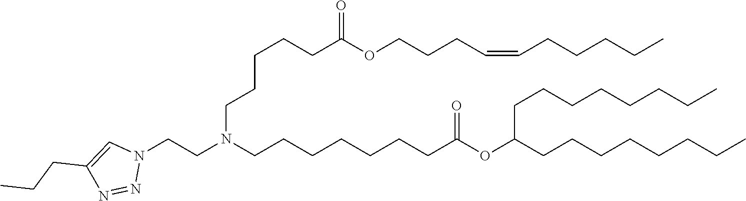 [SMILES: CCCCC/C=C\CCCOC(=O)CCCCCN(CCCCCCCC(=O)OC(CCCCCCCC)CCCCCCCC)CCn1cc(CCC)nn1] |
| LipidB23-11 | 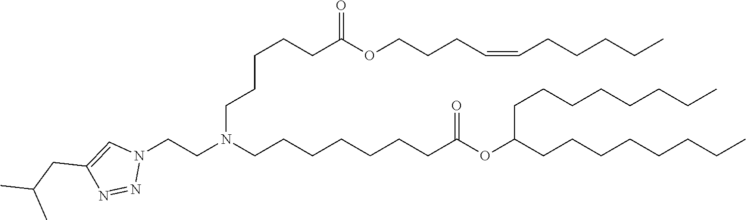 [SMILES: CCCCC/C=C\CCCOC(=O)CCCCCN(CCCCCCCC(=O)OC(CCCCCCCC)CCCCCCCC)CCn1cc(CC(C)C)nn1] |
| LipidB23-12 | 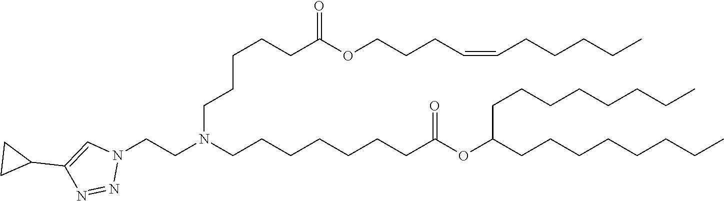 [SMILES: CCCCC/C=C\CCCOC(=O)CCCCCN(CCCCCCCC(=O)OC(CCCCCCCC)CCCCCCCC)CCn1cc(C2CC2)nn1] |
| LipidB23-13 | 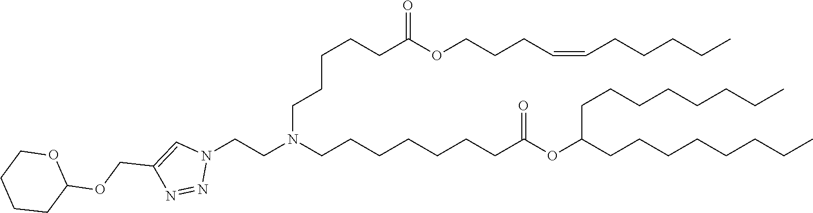 [SMILES: CCCCC/C=C\CCCOC(=O)CCCCCN(CCCCCCCC(=O)OC(CCCCCCCC)CCCCCCCC)CCn1cc(COC2CCCCO2)nn1] |
| LipidB23-14 | 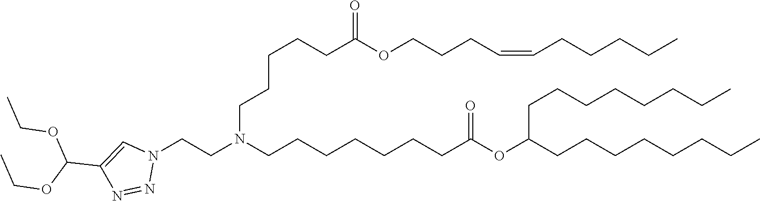 [SMILES: CCCCC/C=C\CCCOC(=O)CCCCCN(CCCCCCCC(=O)OC(CCCCCCCC)CCCCCCCC)CCn1cc(C(OCC)OCC)nn1] |
| LipidB23-15 | 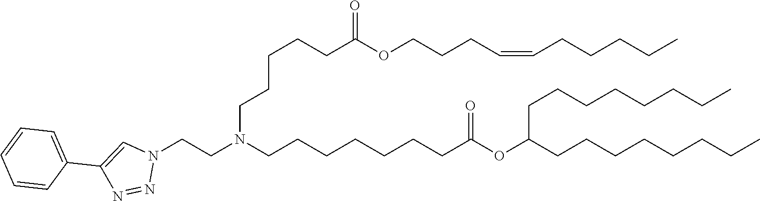 [SMILES: CCCCC/C=C\CCCOC(=O)CCCCCN(CCCCCCCC(=O)OC(CCCCCCCC)CCCCCCCC)CCn1cc(-c2ccccc2)nn1] |

The present disclosure has the following beneficial effects:
1. The compounds provided by the present disclosure all contain triazole linkers, which have good biocompatibility and low toxicity.
2. The compounds provided by the present disclosure can form uniform lipid nanoparticles with high entrapped efficiency, a particle size of about 100 nm and high delivery efficiency with the phospholipid, the structural lipid and the PEGylation lipid.
3. The compound of formula (I) is synthesized in the present disclosure by using a CuAAC reaction in combination with simple addition and esterification reactions, and the synthesis is simple and rapid.

### Definition

When a numerical range is listed, each value and sub-range within the range are intended to be included. For example, “C₁₋₆ alkyl” includes C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅ and C₅₋₆ alkyl.

The term “alkyl” refers to a straight or branched saturated hydrocarbon group comprising one or more carbon atoms (e.g., 1, 2, 3, 4, 5, 6, 7, 8, 9, 10, 11, 12, 13, 14, 15, 16, 17, 18, 19, 20 or more carbon atoms). For example, “C₁₋₄ alkyl” refers to an optionally substituted straight or branched saturated hydrocarbon group comprising 1-4 carbon atoms. “C₅₋₁₀ alkyl” refers to an optionally substituted straight or branched saturated hydrocarbon group comprising 5-10 carbon atoms. Unless otherwise specified, the alkyl described herein refers to unsubstituted or substituted alkyl.

The term “alkenyl” refers to a straight or branched hydrocarbon radical containing from 4 to 18 carbon atoms and at least one carbon-carbon double bond. Exemplary such groups comprise vinyl or allyl. For example, “C₂₋₆ alkenyl” refers to a straight and branched alkenyl having 2 to 6 carbon atoms.

The term “C₃₋₆ cycloalkyl” refers to a non-aromatic cyclic hydrocarbon group having 3 to 6 ring carbon atoms. The exemplary cycloalkyl includes, but is not limited to: cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), etc. The cycloalkyl group may be optionally substituted with one or more substituents.

The term “4- to 7-membered heterocyclyl” refers to a group of a 4- to 7-membered non-aromatic ring system having ring carbon atoms and 1 to 2 ring heteroatoms, wherein each heteroatom is independently selected from nitrogen, oxygen, sulfur, boron, phosphorus and silicon. In the heterocyclyl containing one or more nitrogen atoms, a connection point may be a carbon or nitrogen atom, as valency permits. Exemplary 4-membered heterocyclyl containing one heteroatom includes, but is not limited to: azetidinyl, oxetanyl and thietanyl. Exemplary 5-membered heterocyclyl containing one heteroatom includes, but is not limited to: tetrahydrofuranyl, dihydrofuranyl, tetrahydrothienyl, dihydrothienyl, pyrrolidinyl, dihydropyrrolyl and pyrrolyl-2,5-dione. Exemplary 5-membered heterocyclyl containing two heteroatoms includes, but is not limited to: dioxolanyl, oxasulfuranyl, disulfuranyl and oxazolidin-2-one. Exemplary 6-membered heterocyclyl containing one heteroatom includes, but is not limited to: piperidinyl, tetrahydropyranyl, dihydropyridinyl and thianyl. Exemplary 6-membered heterocyclyl containing two heteroatoms includes, but is not limited to: piperazinyl, morpholinyl, dithianyl and dioxanyl. Exemplary 7-membered heterocyclyl containing one heteroatom includes, but is not limited to: azepanyl, oxepanyl and thiepanyl.

The term “isomer” refers to different compounds that have the same molecular formula. The present disclosure particularly relates to a stereoisomer, and the term “stereoisomer” is an isomer that differs only in the arrangement of atoms in space.

In some cases, the compounds of the present disclosure may form salts, which are also within the scope of the present disclosure. The term “salt(s)” refers to acidic and/or basic salts formed with inorganic and/or organic acids and bases. The present disclosure particularly relates to a pharmaceutically acceptable salt.

The term “halogen” refers to F, Cl, Br and I.

The term “aryl” refers to an aromatic ring group containing 6 to 10 ring carbons. Examples include phenyl and naphthyl.

The term “heteroaryl” refers to an aromatic ring system containing 5 to 14 aromatic ring atoms which may be a single ring, two fused rings or three fused rings, wherein at least one aromatic ring atom is a heteroatom selected from, but not limited to, the group consisting of O, S and N. Examples include furyl, thienyl, pyrrolyl, imidazolyl, oxazolyl, thiazolyl, isoxazolyl, pyrazolyl, isothiazolyl, oxadiazolyl, triazolyl, thiadiazolyl, pyridinyl, pyrazinyl, pyrimidinyl, pyridazinyl, triazinyl, etc. Examples further include carbazolyl, quinolizinyl, quinolinyl, isoquinolinyl, cinnolinyl, phthalazinyl, quinazolinyl, quinoxalinyl, triazinyl, indolyl, isoindolyl, indazolyl, indolizinyl, purinyl, naphthyridinyl, pteridinyl, acridinyl, phenazinyl, phenothiazinyl, phenoxazinyl, benzoxazolyl, benzothiazolyl, 1H-benzimidazolyl, imidazopyridinyl, benzothienyl, benzofuranyl, isobenzofuran, etc.

### BRIEF DESCRIPTION OF THE DRAWINGS

FIG. 1 shows a method for constructing the ionizable lipid molecule A library.

FIG. 2 shows a method for constructing the ionizable lipid molecule B library.

FIGS. 3A to 3C show the cytotoxicity analysis of LipidA-X, LipidB22-X, and LipidB23-X on Hela cells, respectively.

FIGS. 4A to 4C show the particle size and distribution of LA-X, LB22-X, and LB23-X, respectively.

FIG. 5 is a thermogram comparing the ζ-potential and structure of LNPs.

FIG. 6 is a thermogram comparing the entrapped efficiency and structure of LNPs.

FIGS. 7A to 7C show the luciferase mRNA delivery efficiencies of LA-X, LB22-X, and LB23-X, respectively.

FIG. 8 is a thermogram comparing the luciferase mRNA delivery efficiency and structure of LNPs.

FIG. 9 shows the summary and comparison of the properties of LNPs.

FIG. 10 shows the in vivo delivery efficiency of lipid nanoparticles (LNPs) containing LB23-7.

FIG. 11 shows the in vivo tissue distribution of lipid nanoparticles (LNPs) containing LB23-7 (organs from top to bottom are heart, liver, spleen, lung and kidney, respectively).

### DETAILED DESCRIPTION OF EMBODIMENTS

To make the objectives and technical solutions of the present disclosure clearer, the present disclosure will be further described in detail by the following embodiments in combination with the accompanying drawings.

Experimental instruments: Nuclear magnetic resonance spectrum (1H-NMR): Bruker AVANCE III-400 400 MHz, Chloroform-d is used as the sample solvent. The chemical shift 8 is expressed in ppm; and the coupling constant J is expressed in Hz. In the NMR spectrum, s represents singlet, d represents doublet, t represents triplet, and m represents multiplet. Mass spectrometer: LC MS-2020 resolution mass spectrometer; ultrapure water instrument: Millipore Milli-Q-Integral is used to prepare ultrapure water for experiments; microplate reader: TECAN Spark 10M multi-function microplate reader; pH meter: METTLER TOLEDO FiveEasy Plus™ benchtop pH meter; shaker: Kylin-Bell Lab Instruments ZD-9550 shaker; liposome extruder: LiposoFast-Basic LF-1 liposome preparation extruder; and dynamic light scattering instrument: BrookHaven 90plus PALS dynamic light scattering instrument.

Experimental reagents: Quant-iT™ RiboGreen™ RNA Assay Kit (invitrogen) is purchased from Thermo Fisher Scientific; Luciferase Reporter Gene Assay Kit is purchased from Yeasen Biotech; liposome adjuvants are purchased from AVT (Shanghai) Pharmaceutical Technology Co., Ltd.; Cell Counting Kit-8 (CCK-8) is purchased from Coolaber; deuterated chloroform is purchased from Macklin; luciferase mRNA is provided by Vazyme; conventional solvents are purchased from Energy Chemical and are of analytical grade; and all raw materials are purchased from Bidepharm and are of analytical grade.

### Example 1: Synthesis of SM-102

The preparation method of SM-102 comprises the following steps:

### Step 1: Synthesis of Tail-2

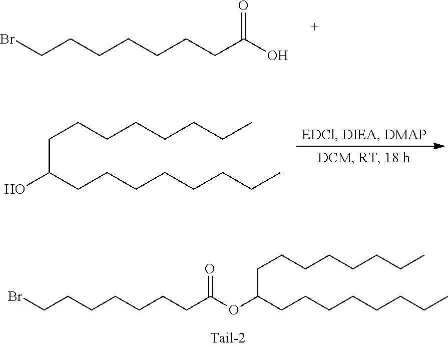 [SMILES: O=C(O)CCCCCCCBr ; CCCCCCCCC(O)CCCCCCCC ; "DCM, RT, 18 h" ; "EDCl, DIEA, DMAP" ; CCCCCCCCC(CCCCCCCC)OC(=O)CCCCCCCBr]

8-Bromooctanoic acid (10.0008 g, 0.0448 mol) was added to a round-bottom flask and dissolved in DCM, and 9-heptadecanol (12.6458 g, 0.0493 mol), EDCI (12.8822 g, 0.0672 mol), DIEA (14.4890 g, 0.1121 mol) and DMAP (0.8214 g, 0.0067 mol) were added. The mixture was reacted at room temperature for 18 h with stirring. The reaction was monitored by TLC. After the reaction was completed, the solvent was concentrated by evaporation, redissolved in EA, and washed three times with 3% KHSO4 solution. The upper organic phase was collected and dried over anhydrous sodium sulfate for 30 minutes. The organic phase was filtered, concentrated by evaporation, mixed with silica gel, and purified by silica gel column chromatography in an elution system of PE:EA=100:1. The product was collected to obtain a colorless oily liquid (12.0968 g) with a yield of 58.5%. An appropriate amount of the product was dissolved in deuterated chloroform CDCl₃ for NMR characterization: ¹H NMR (400 MHZ, Chloroform-d) δ 4.87 (p, J=6.3 Hz, 1H), 3.40 (t, J=6.8 Hz, 2H), 2.28 (t, J=7.4 Hz, 2H), 1.85 (p, J=6.9 Hz, 2H), 1.63 (dddd, J=12.3, 7.5, 4.7, 2.2 Hz, 2H), 1.54-1.40 (m, 6H), 1.35-1.21 (m, 28H), 0.96-0.81 (m, 6H)

### Step 2: Synthesis of Intermediate 1

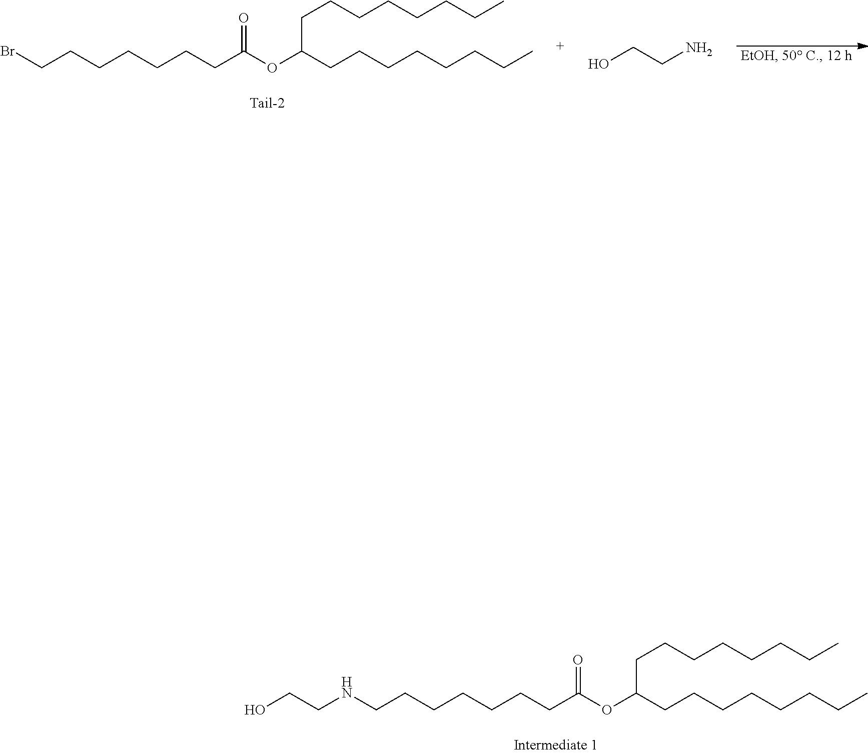

Tail-2 (6.0021 g, 0.0130 mol) was added to a round-bottom flask, followed by the addition of ethanolamine (30 mL) and a small amount of ethanol (6 mL) to facilitate solubilization. The mixture was heated at 50° C. and reacted for 12 h with stirring. The reaction was monitored by TLC. After the reaction was completed, the ethanol was evaporated, the mixture was redissolved in EA, and washed three times with saturated sodium chloride solution. The upper organic phase was collected and dried over anhydrous sodium sulfate for 30 minutes. The dried upper organic phase was filtered, the filtrate was subjected to rotary evaporation under reduced pressure, mixed with silica gel and then purified by silica gel column chromatography using an eluent of PE:EA=5:1. The product was collected to obtain a light yellow oily liquid (4.9247 g) with a yield of 85.7%. An appropriate amount of the product was dissolved in deuterated chloroform CDCl₃ for NMR characterization: ¹H NMR (400 MHZ, Chloroform-d) δ 4.86 (p, J=6.3 Hz, 1H), 3.72-3.58 (m, 2H), 2.87-2.73 (m, 2H), 2.63 (t, J=7.2 Hz, 2H), 2.28 (t, J=7.5 Hz, 2H), 1.62 (p, J=7.2 Hz, 2H), 1.50 (dd, J=7.5, 4.3 Hz, 6H), 1.29 (d, J=27.3 Hz, 30H), 1.01-0.76 (m, 6H). LC-MS: m/z 442.60 (M+H)⁺C₂₇H₅₅NO₃ (441.74).

### Step 3: Synthesis of Tail-1

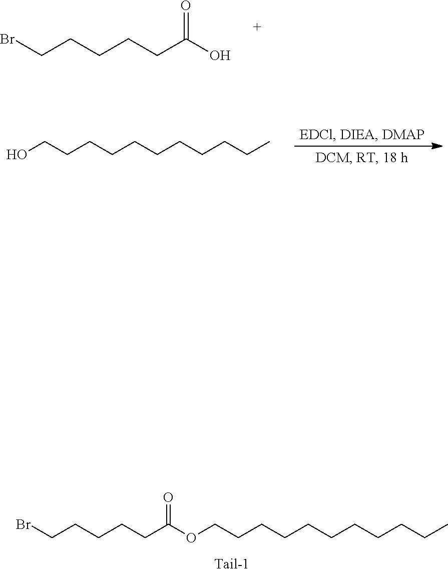 [SMILES: O=C(O)CCCCCBr ; "EDCl, DIEA, DMAP" ; CCCCCCCCCCCO ; "DCM, RT, 18 h" ; CCCCCCCCCCCOC(=O)CCCCCBr]

6-Bromohexanoic acid (10.0067 g, 0.0513 mol) was added to a round-bottom flask and dissolved in DCM. Undecanol (9.7241 g, 0.0564 mol), EDCI (14.7522 g, 0.0769 mol), DIEA (16.5774 g, 0.1283 mol) and DMAP (0.9402 g, 0.0077 mol) were added. The mixture was reacted at room temperature for 18 h with stirring. The reaction was monitored by TLC. After the reaction was completed, the solvent was concentrated by evaporation, redissolved in EA, and washed three times with 3% KHSO4 solution. The upper organic phase was collected and dried over anhydrous sodium sulfate for 30 minutes. The dried upper organic phase was filtered, concentrated by evaporation, mixed with silica gel, and purified by silica gel column chromatography in an elution system of PE:EA=100:1. The product was collected to obtain a colorless oily liquid (10.1135 g) with a yield of 56.4%. An appropriate amount of the product was dissolved in deuterated chloroform CDCl₃ for NMR characterization: ¹H NMR (400 MHZ, Chloroform-d) δ 4.07 (d, J=6.8 Hz, 2H), 3.41 (t, J=6.8 Hz, 2H), 2.32 (t, J=7.4 Hz, 2H), 1.88 (dt, J=14.5, 6.8 Hz, 2H), 1.65 (dq, J=15.7, 8.2, 7.8 Hz, 4H), 1.53-1.44 (m, 2H), 1.28 (d, J=15.7 Hz, 16H), 0.88 (t, J=6.9 Hz, 3H).

### Step 4: Synthesis of SM-102

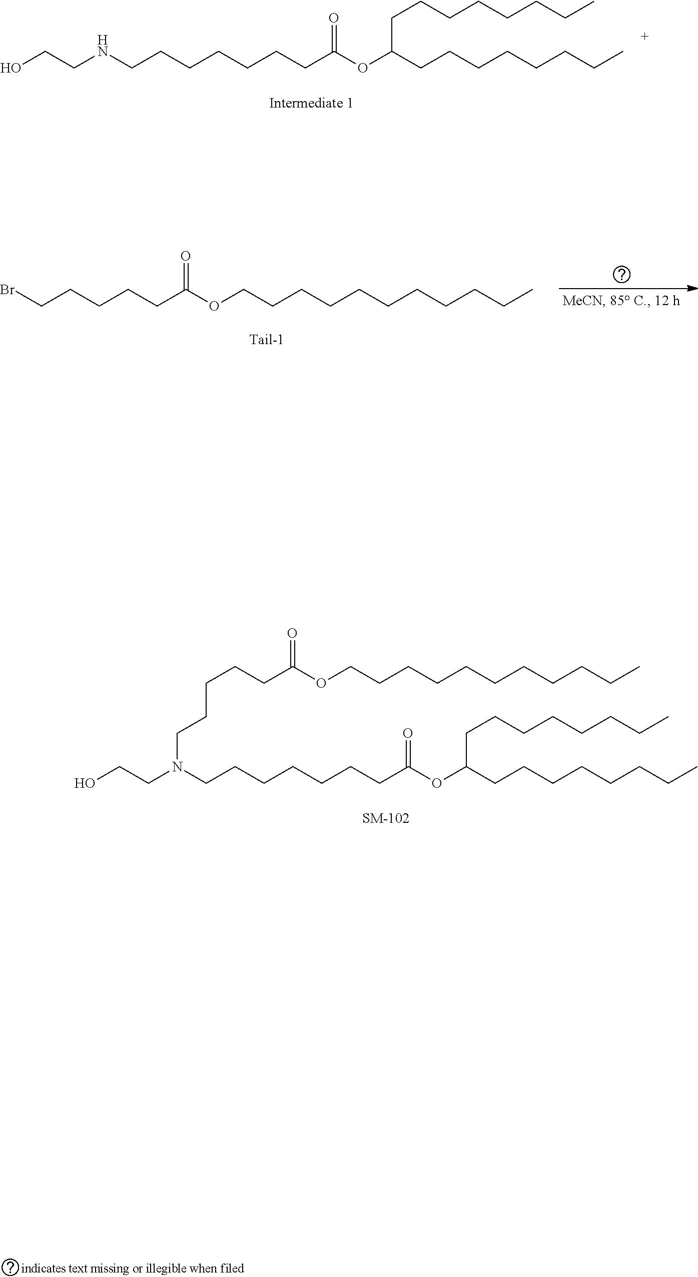

The intermediate 1 (4.9200 g, 0.0111 mol) was added to a round-bottom flask and dissolved in MeCN, and Tail-1 (4.2801 g, 0.0123 mol), K₂CO₃ and KI were added. The mixture was reacted at 85° C. for 12 h with stirring. The reaction was monitored by TLC. After the reaction was completed, MeCN was removed by rotary evaporation, and the residue was redissolved in EA and washed three times with saturated sodium chloride solution. The upper organic layer was collected and dried over anhydrous sodium sulfate for 30 minutes. The upper organic layer was filtered, the filtrate was subjected to rotary evaporation under reduced pressure, mixed with silica gel and then purified by silica gel column chromatography using an eluent of EA:MeOH=10:1. The product was collected to obtain a colorless oily liquid with a yield of 85.7%. An appropriate amount of the product was dissolved in deuterated chloroform CDCl₃ for NMR characterization: ¹H NMR (400 MHZ, Chloroform-d) δ 4.86 (p, J=6.3 Hz, 1H), 4.17-4.03 (m, 4H), 3.61 (t, J=5.3 Hz, 2H), 2.67 (t, J=5.3 Hz, 2H), 2.60-2.50 (m, 4H), 2.29 (dt, J=10.6, 7.4 Hz, 4H), 1.62 (dq, J=9.9, 7.2, 6.7 Hz, 6H), 1.55-1.44 (m, 8H), 1.35-1.22 (m, 53H), 0.99-0.84 (m, 9H). LC-MS: m/z 710.80 (M+H)⁺C₄₄H₈₇NO₅ (710.18).

### Example 2 Construction and Characterization of Ionizable Lipid Molecule a Library

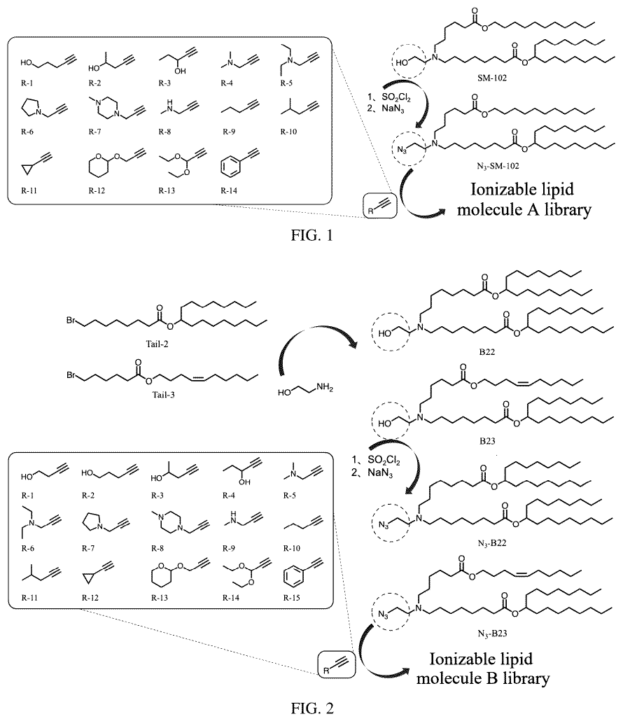

The ionizable lipid A library was constructed based on the structure of SM-102. The head structure of SM-102 was structurally modified by a CuAAC reaction to obtain a series of novel ionizable lipids with different head structures. The preparation method of the head structure (R—X) and the ionizable lipid molecule A is shown in FIG. 1.

The preparation method specifically comprises the following steps:

### Step 1: Synthesis of N3-SM-102 (azide tail skeleton)

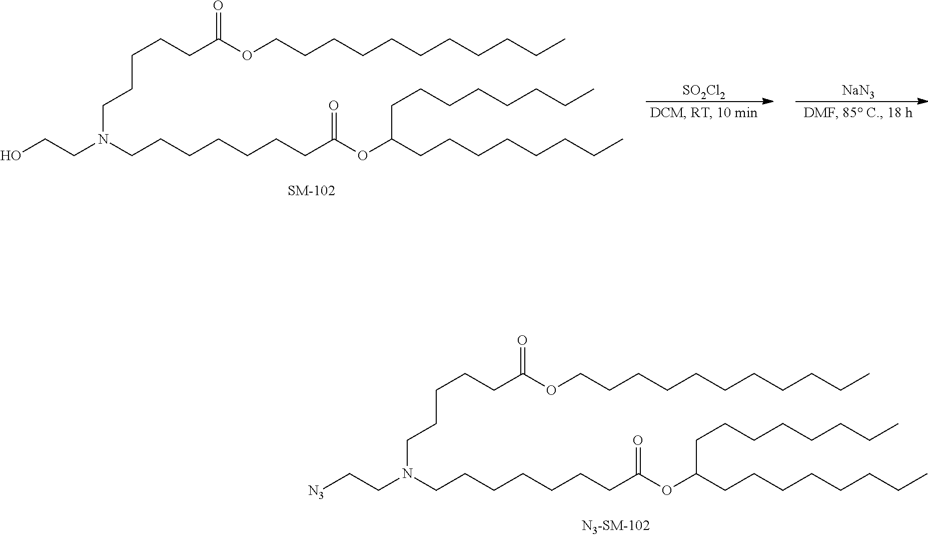 [SMILES: CCCCCCCCCCCOC(=O)CCCCCN(CCO)CCCCCCCC(=O)OC(CCCCCCCC)CCCCCCCC ; "SO2Cl2" ; "DCM, RT, 10 min" ; "DMF, 85° C., 18 h" ; CCCCCCCCCCCOC(=O)CCCCCN(CCCCCCCC(=O)OC(CCCCCCCC)CCCCCCCC)CCN=[N+]=[N-] ; "NaN3"]

SM-102 (4.9200 g, 0.0069 mol) was added to a round-bottom flask and dissolved in DCM. SO₂Cl₂ (2.8059 g, 0.0208 mol) was added dropwise at room temperature with stirring. After the addition was completed, the mixture was reacted at room temperature for 10 min with stirring. The reaction was monitored by TLC. After the reaction was completed, the reaction was stopped, and the reaction solution was washed three times with a saturated sodium bicarbonate solution to remove the acid and make the reaction solution system alkaline. The lower organic layer was collected, dried over anhydrous sodium sulfate and filtered. The filtrate was subjected to rotary evaporation under reduced pressure to obtain crude product Cl-SM-102. The crude product Cl-SM-102 was directly dissolved in DMF, and an aqueous solution of NaN₃ (0.8971 g, 0.0138 mol) was added dropwise while stirring, and the mixture was stirred at room temperature for 10 min. The reaction was then transferred to an oil bath and stirred at 85° C. for 18 h. The reaction was monitored by TLC. After the reaction was completed, the reaction was stopped, DMF was removed by rotary evaporation under reduced pressure, and the residue was redissolved in EA and washed three times with saturated sodium chloride solution. The upper organic layer was collected and dried over anhydrous sodium sulfate for 30 minutes. The dried upper organic layer was filtered, the filtrate was subjected to rotary evaporation under reduced pressure, mixed with silica gel and then purified by silica gel column chromatography using an eluent of PE:EA=50:1. The product was collected to obtain a light yellow oily liquid (3.8792 g) with a yield of 76.5%. An appropriate amount of the product was dissolved in deuterated chloroform CDCl₃ for NMR characterization: ¹H NMR (400 MHZ, Chloroform-d) δ 4.86 (p, J=6.3 Hz, 1H), 4.06 (t, J=6.7 Hz, 2H), 3.24 (t, J=6.2 Hz, 2H), 2.63 (t, J=6.2 Hz, 2H), 2.54-2.37 (m, 4H), 2.29 (dt, J=9.3, 7.5 Hz, 4H), 1.69-1.57 (m, 8H), 1.55-1.39 (m, 9H), 1.37-1.21 (m, 49H), 0.88 (td, J=6.9, 1.6 Hz, 9H). LC-MS: m/z 735.60 (M+H)⁺C₄₄H₈₆N₄O₄ (735.20).

### Step 2: Preparation of LipidA-X (CuAAC method)

The synthesis steps of LipidA-1 to LipidA-14 are as follows:

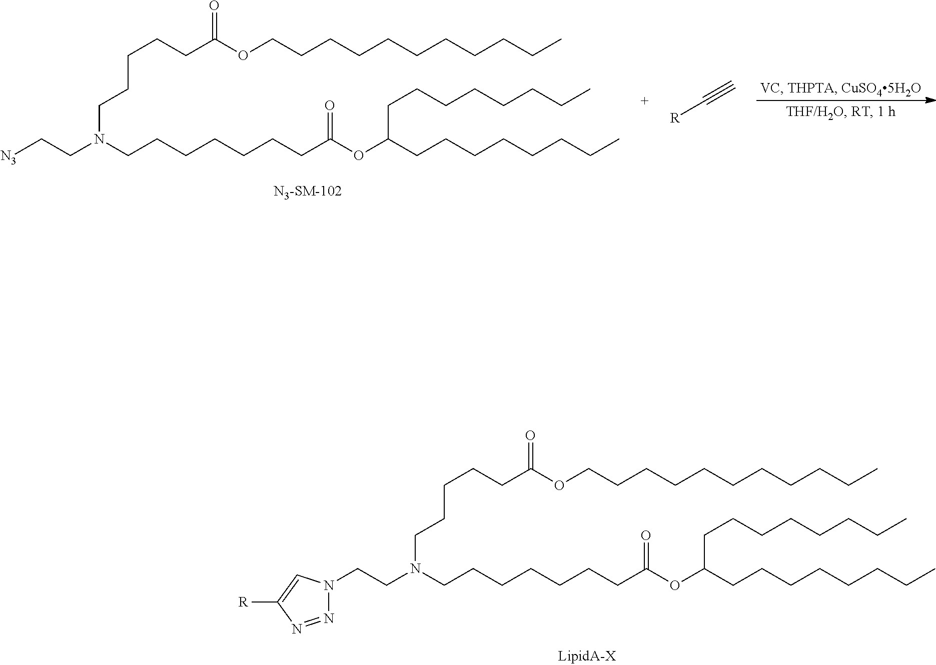

N₃-SM-102, VC, THPTA, CuSO₄ and the head small molecule R—X of a terminal alkyne were weighed based on the equivalent weight in Table 1, dissolved in the corresponding solvents, and added into a flask in the order of N₃-SM-102, VC, THPTA, CuSO₄ and R—X. The solvent system was adjusted to THF:H₂O:DMSO=4:1:0.05. The mixture was reacted at room temperature for 1 hour with stirring. The reaction was monitored by TLC. After the reaction was completed, the reaction solution was evaporated to dryness under reduced pressure, redissolved in EA, and washed 5 times with saturated sodium chloride solution to obtain pure product LipidA-X without further purification by silica gel column chromatography.

TABLE 1
Feed ratio and usage amount details for synthesis of LipidA-X

| | N₃-SM-102 | VC | THPTA | CuSO₄•5H₂O |
|---|---|---|---|---|
| M | 735.20 | 176.13 | 434.25 | 249.68 |
| eq | 1 | 1 | 0.1 | 1 |
| m/mg | 100.0 | 23.9 | 5.9 | 34.0 |

| | R-1 | R-2 | R-3 | R-4 | R-5 |
|---|---|---|---|---|---|
| M | 84.12 | 84.12 | 84.12 | 83.13 | 111.19 |
| eq | 1.2 | | | | |
| m/mg | 13.7 | 13.7 | 13.7 | 13.6 | 18.1 |

| | R-6 | R-7 | R-8 | R-9 | R-10 |
|---|---|---|---|---|---|
| M | 109.17 | 138.21 | 69.11 | 68.12 | 82.15 |
| eq | 1.2 | | | | |
| m/mg | 17.8 | 22.6 | 11.3 | 11.1 | 13.4 |

| | R-11 | R-12 | R-13 | R-14 |
|---|---|---|---|---|
| M | 66.10 | 140.18 | 128.17 | 102.14 |

| | |
|---|---|
| eq | 1.2 |

| | | | | |
|---|---|---|---|---|
| m/mg | 10.8 | 22.9 | 20.9 | 16.7 |

The obtained products and characterizations thereof are as follows:

LipidA-1: ¹H NMR (400 MHZ, Chloroform-d) δ 7.43 (s, 1H), 4.86 (p, J=6.3 Hz, 1H), 4.35 (t, J=6.1 Hz, 2H), 4.05 (t, J=6.8 Hz, 2H), 3.70 (t, J=6.1 Hz, 2H), 2.85 (dt, J=14.5, 6.7 Hz, 4H), 2.46-2.39 (m, 4H), 2.31-2.25 (m, 4H), 1.93 (p, J=7.1 Hz, 2H), 1.61 (ddd, J=11.7, 7.4, 4.5 Hz, 7H), 1.53-1.47 (m, 5H), 1.28 (d, J=14.6 Hz, 64H), 0.93-0.80 (m, 12H). ¹³C NMR (101 MHZ, Chloroform-d) δ 173.75 (d, J=16.4 Hz), 74.18, 64.54, 61.84, 54.29 (d, J=18.5 Hz), 34.67, 34.20 (d, J=13.5 Hz), 32.20-31.74 (m), 29.98-28.95 (m), 28.64, 27.38-26.50 (m), 25.92, 25.31, 25.08, 24.86, 22.67 (d, J=2.0 Hz), 22.11, 14.10. LC-MS: m/z 820.20 (M+H)⁺C₄₉H₉₄N₄O₅ (819.31).

LipidA-2: ¹H NMR (400 MHZ, Chloroform-d) δ 7.49 (s, 1H), 4.86 (p, J=6.3 Hz, 1H), 4.35 (t, J=6.2 Hz, 2H), 4.18-4.11 (m, 1H), 4.05 (t, J=6.8 Hz, 2H), 2.93-2.71 (m, 4H), 2.42 (td, J=7.4, 2.6 Hz, 4H), 2.28 (td, J=7.5, 1.6 Hz, 4H), 1.61 (ddt, J=12.6, 7.7, 4.4 Hz, 7H), 1.53-1.47 (m, 4H), 1.41-1.15 (m, 56H), 0.88 (td, J=6.9, 1.8 Hz, 9H). ¹³C NMR (101 MHZ, Chloroform-d) δ 173.67 (d, J=16.3 Hz), 144.84, 122.58, 74.10, 64.45, 54.31, 54.16, 54.12, 48.78, 34.84, 34.62, 34.22, 34.11, 31.87, 31.83, 29.56, 29.55, 29.50, 29.47, 29.30, 29.22, 29.20, 29.14, 28.62, 27.21, 27.04, 26.86, 25.90, 25.28, 25.04, 24.83, 22.83, 22.64, 22.63, 14.07. LC-MS: m/z 820.25 (M+H)⁺C₄₉H₉₄N₄O₅ (819.31).

LipidA-3: ¹H NMR (400 MHZ, Chloroform-d) δ 7.59 (s, 1H), 4.91-4.79 (m, 2H), 4.36 (t, J=6.2 Hz, 2H), 4.05 (t, J=6.8 Hz, 2H), 2.86 (t, J=6.1 Hz, 2H), 2.42 (t, J=7.3 Hz, 4H), 2.28 (t, J=7.5 Hz, 4H), 1.89 (ddq, J=21.0, 13.8, 7.3 Hz, 3H), 1.65-1.56 (m, 7H), 1.28 (dd, J=14.7, 6.0 Hz, 57H), 0.99 (t, J=7.4 Hz, 3H), 0.88 (td, J=7.0, 1.9 Hz, 10H). ¹³C NMR (101 MHZ, Chloroform-d) δ 173.72 (d, J=21.4 Hz), 121.30, 74.11, 64.49, 54.28, 54.11, 54.08, 48.80, 34.62, 34.22, 34.10, 31.87, 31.83, 30.39, 29.57, 29.55, 29.50, 29.47, 29.30, 29.22, 29.20, 29.13, 28.61, 27.20, 26.97, 26.83, 26.79, 25.89, 25.28, 25.04, 24.81, 22.64, 22.63, 14.07, 9.76. LC-MS: m/z 820.20 (M+H)⁺C₄₉H₉₄N₄O₅ (819.31).

LipidA-4: ¹H NMR (400 MHZ, Chloroform-d) δ 4.86 (p, J=6.3 Hz, 2H), 3.53 (t, J=5.4 Hz, 2H), 2.58 (t, J=5.4 Hz, 2H), 2.48-2.40 (m, 4H), 2.28 (t, J=7.5 Hz, 4H), 1.68-57 (m, 4H), 1.55-1.39 (m, 13H), 1.27 (d, J=8.3 Hz, 58H), 0.93-0.84 (m, 12H). ¹³C NMR (101 MHZ, Chloroform-d) δ 173.70 (d, J=14.7 Hz), 77.34, 74.14, 64.50, 54.30 (d, J=18.7 Hz), 34.69, 34.22 (d, J=14.8 Hz), 29.72-28.89 (m), 28.66, 27.21 (d, J=14.8 Hz), 26.93, 25.56-24.87 (m). LC-MS: m/z 819.10 (M+H)⁺C₄₉H₉₅N₅O₄ (818.33).

LipidA-5: ¹H NMR (400 MHZ, Chloroform-d) δ 7.62 (s, 1H), 4.86 (p, J=6.3 Hz, 1H), 4.36 (t, J=6.4 Hz, 2H), 4.05 (t, J=6.8 Hz, 2H), 3.84 (s, 2H), 2.86 (t, J=6.3 Hz, 2H), 2.59 (d, J=6.5 Hz, 3H), 2.48-2.36 (m, 3H), 2.28 (td, J=7.5, 2.3 Hz, 4H), 1.97-1.56 (m, 12H), 1.54-1.46 (m, 4H), 1.27 (d, J=14.7 Hz, 50H), 1.12 (t, J=7.1 Hz, 5H), 0.94-0.80 (m, 9H). ¹³C NMR (101 MHZ, Chloroform-d) δ 173.70 (d, J=14.7 Hz), 74.14, 64.50, 54.30 (d, J=18.7 Hz), 34.69, 34.22 (d, J=14.8 Hz), 29.72-28.89 (m), 28.66, 27.21 (d, J=14.8 Hz), 26.93, 25.56-24.87 (m). LC-MS: m/z 846.90 (M+H)⁺C₅₁H₉₉N₅O₄ (846.38).

LipidA-6: ¹H NMR (400 MHZ, Chloroform-d) δ 8.02 (s, 1H), 4.86 (p, J=6.2 Hz, 1H), 4.38 (t, J=5.5 Hz, 2H), 4.05 (t, J=6.8 Hz, 2H), 3.01 (s, 3H), 2.88 (t, J=5.3 Hz, 2H), 2.48-2.37 (m, 4H), 2.28 (t, J=7.5 Hz, 4H), 1.99 (s, 4H), 1.61 (h, J=9.5, 7.9 Hz, 7H), 1.54-1.46 (m, 4H), 1.26 (s, 54H), 0.91-0.83 (m, 9H). ¹³C NMR (101 MHZ, Chloroform-d) δ 173.71 (d, J=14.6 Hz), 74.13, 64.49, 54.25 (d, J=18.6 Hz), 53.45, 34.70, 34.23 (d, J=16.9 Hz), 31.89 (d, J=4.4 Hz), 29.71-29.08 (m), 28.67, 27.39-26.88 (m), 25.94, 25.33, 25.13, 24.91, 22.68 (d, J=1.8 Hz), 14.12. LC-MS: m/z 844.90 (M+H)⁺C₅₁H₉₇N₅O₄ (844.37).

LipidA-7: ¹H NMR (400 MHZ, Chloroform-d) δ 7.50 (s, 1H), 4.79 (p, J=6.3 Hz, 1H), 4.28 (t, J=6.4 Hz, 2H), 3.98 (t, J=6.8 Hz, 2H), 3.62 (s, 2H), 2.78 (t, J=6.4 Hz, 2H), 2.40-2.30 (m, 6H), 2.25-2.17 (m, 8H), 1.54 (pd, J=7.4, 3.5 Hz, 6H), 1.43 (q, J=6.1 Hz, 5H), 1.35-1.16 (m, 56H), 0.81 (td, J=6.9, 1.9 Hz, 11H). ¹³C NMR (101 MHZ, Chloroform-d) δ 173.70 (d, J=14.7 Hz), 74.14, 64.50, 54.30 (d, J=18.7 Hz), 34.69, 34.22 (d, J=14.8 Hz), 29.72-28.89 (m), 28.66, 27.21 (d, J=14.8 Hz), 26.93, 25.56-24.87 (m). LC-MS: m/z 873.30 (M+H)⁺C₅₂H₁₀₀N₆₀₄ (872.41).

LipidA-8: ¹H NMR (400 MHZ, Chloroform-d) δ 6.00-5.68 (m, 1H), 4.84 (p, J=6.2 Hz, 1H), 4.04 (q, J=6.1 Hz, 2H), 3.92 (d, J=5.3 Hz, 1H), 3.40 (s, 1H), 2.55-2.36 (m, 3H), 2.27 (dt, J=14.6, 7.5 Hz, 4H), 1.54-1.41 (m, 5H), 1.24 (s, 49H), 0.85 (d, J=7.4 Hz, 12H). ¹³C NMR (101 MHz, Chloroform-d) δ 173.59, 135.28, 115.99, 74.09 (d, J=2.5 Hz), 72.24, 68.94, 64.92-64.15 (m), 61.55, 53.57, 34.12, 31.86 (d, J=4.2 Hz), 29.52 (dd, J=6.1, 4.0 Hz), 29.27 (d, J=9.3 Hz), 28.66 (d, J=6.1 Hz), 26.08-25.72 (m), 25.30 (d, J=2.0 Hz), 25.11 (d, J=4.8 Hz), 22.65 (d, J=2.0 Hz). LC-MS: m/z 804.95 (M+H)⁺C₄₈H₉₃N₅O₄ (804.30).

LipidA-9: ¹H NMR (400 MHZ, Chloroform-d) δ 7.37 (s, 1H), 4.86 (p, J=6.3 Hz, 1H), 4.33 (t, J=6.3 Hz, 2H), 4.05 (t, J=6.8 Hz, 2H), 2.84 (t, J=6.3 Hz, 2H), 2.69 (t, J=7.6 Hz, 2H), 2.41 (dq, J=7.8, 4.6 Hz, 4H), 2.28 (td, J=7.5, 1.8 Hz, 4H), 1.70 (dt, J=15.0, 7.4 Hz, 3H), 1.61 (qq, J=7.5, 4.4, 3.2 Hz, 6H), 1.50 (d, J=6.1 Hz, 2H), 1.41-1.19 (m, 54H), 0.97 (t, J=7.4 Hz, 3H), 0.88 (td, J=6.9, 1.9 Hz, 9H). ¹³C NMR (101 MHZ, Chloroform-d) δ 173.67 (d, J=14.6 Hz), 147.82, 121.42, 74.11, 64.47, 54.74-53.85 (m), 48.71, 34.67, 34.20 (d, J=12.9 Hz), 31.88 (d, J=4.2 Hz), 29.79-28.14 (m), 27.69, 27.44-26.54 (m), 25.92, 25.31, 24.98 (d, J=22.8 Hz), 22.96-22.54 (m), 14.10, 13.78. LC-MS: m/z 873.30 (M+H)⁺C₅₂H₁₀₀N₆₀₄ (872.41). LC-MS: m/z 803.95 (M+H)+C₄₉H₉₄N₄O₄ (803.32).

LipidA-10: ¹H NMR (400 MHZ, Chloroform-d) δ 7.37 (s, 1H), 4.86 (p, J=6.2 Hz, 1H), 4.34 (t, J=6.3 Hz, 2H), 4.05 (t, J=6.8 Hz, 2H), 2.85 (t, J=6.3 Hz, 2H), 2.58 (d, J=7.0 Hz, 2H), 2.42 (td, J=7.5, 4.5 Hz, 4H), 2.28 (ddd, J=7.6, 6.3, 1.8 Hz, 4H), 1.96 (dt, J=13.5, 6.7 Hz, 1H), 1.85-1.74 (m, 2H), 1.61 (qq, J=7.4, 4.8, 3.6 Hz, 7H), 1.50 (d, J=6.6 Hz, 3H), 1.27 (p, J=8.7, 7.5 Hz, 58H), 0.94 (d, J=6.6 Hz, 6H), 0.88 (td, J=6.9, 1.9 Hz, 10H). ¹³C NMR (101 MHZ, Chloroform-d) δ 173.65 (d, J=14.5 Hz), 146.81, 126.90, 74.10, 64.46, 54.63-53.64 (m), 48.71, 34.72 (d, J=11.7 Hz), 34.20 (d, J=12.5 Hz), 31.88 (d, J=4.3 Hz), 29.76-29.05 (m), 28.70 (d, J=10.8 Hz), 27.44-26.04 (m), 25.92, 25.20 (d, J=21.7 Hz), 24.86, 22.66 (d, J=1.8 Hz), 22.30, 14.10. LC-MS: m/z 817.90 (M+H)⁺C₅₀H₉₆N₄O₄ (817.34).

LipidA-11: ¹H NMR (400 MHZ, Chloroform-d) δ 7.32 (s, 1H), 4.86 (p, J=6.3 Hz, 1H), 4.30 (t, J=6.3 Hz, 2H), 4.05 (t, J=6.8 Hz, 2H), 2.82 (t, J=6.3 Hz, 2H), 2.41 (td, J=7.4, 4.4 Hz, 4H), 2.28 (td, J=7.5, 3.1 Hz, 4H), 1.94 (tt, J=8.4, 5.0 Hz, 1H), 1.70-1.54 (m, 11H), 1.50 (d, J=6.5 Hz, 4H), 1.43-1.19 (m, 56H), 0.98-0.84 (m, 12H). ¹³C NMR (101 MHZ, Chloroform-d) δ 173.66 (d, J=15.5 Hz), 149.80, 120.42, 74.09, 64.45, 54.70-53.93 (m), 48.78, 34.66, 34.20 (d, J=13.4 Hz), 31.87 (d, J=4.2 Hz), 29.82-28.85 (m), 28.65, 27.49-26.53 (m), 25.92, 25.31, 25.10, 24.87, 22.66 (d, J=2.0 Hz), 14.09, 7.63, 6.68. LC-MS: m/z 801.80 (M+H)⁺C₄₉H₉₂N₄O₄ (801.30).

LipidA-12: ¹H NMR (400 MHz, Chloroform-d) δ 7.65 (s, 1H), 4.90-4.81 (m, 2H), 4.75 (t, J=3.4 Hz, 1H), 4.65 (d, J=12.3 Hz, 1H), 4.37 (t, J=5.8 Hz, 2H), 4.05 (t, J=6.8 Hz, 2H), 2.88 (s, 2H), 2.44 (s, 4H), 2.31-2.24 (m, 5H), 1.61 (dt, J=10.5, 5.4 Hz, 9H), 1.40-1.21 (m, 61H), 0.88 (t, J=6.8 Hz, 11H). ¹³C NMR (101 MHZ, Chloroform-d) δ 173.65 (d, J=13.9 Hz), 123.30, 98.06, 74.11, 64.47, 62.26, 54.39, 54.19, 54.15, 34.66, 34.25, 34.14, 31.89, 31.85, 30.48, 29.59, 29.57, 29.52, 29.49, 29.32, 29.23, 29.19, 28.65, 27.26, 26.90, 25.92, 25.40, 25.31, 25.09, 24.85, 19.38, 14.10. LC-MS: m/z 876.30 (M+H)⁺C₅₂H₉₈N₄O₆ (875.38).

LipidA-13: ¹H NMR (400 MHZ, Chloroform-d) δ 7.68 (s, 1H), 5.71 (s, 1H), 4.86 (p, J=6.3 Hz, 1H), 4.38 (h, J=7.0 Hz, 2H), 4.05 (t, J=6.8 Hz, 2H), 3.74-3.53 (m, 3H), 2.86 (d, J=6.5 Hz, 2H), 2.48-2.37 (m, 3H), 2.28 (td, J=7.5, 3.0 Hz, 4H), 1.61 (th, J=7.5, 4.0, 2.9 Hz, 6H), 1.50 (q, J=6.0 Hz, 4H), 1.43-1.14 (m, 59H), 0.88 (t, J=6.7 Hz, 9H). ¹³C NMR (101 MHZ, Chloroform-d) δ 173.66 (d, J=13.9 Hz), 96.93, 74.12, 64.48, 61.55, 54.38, 54.19, 34.67, 34.26, 34.14, 31.90, 31.86, 29.58, 29.53, 29.50, 29.33, 29.26, 29.23, 29.19, 28.65, 27.25, 26.90, 25.93, 25.32, 25.11, 24.85, 22.68, 22.66, 15.17, 14.10. LC-MS: m/z 863.95 (M+H)⁺C₅₁H₉₈N₄O₆ (863.37).

LipidA-14: ¹H NMR (400 MHZ, Chloroform-d) δ 7.89 (s, 1H), 7.83 (dd, J=7.2, 1.6 Hz, 2H), 7.42 (t, J=7.6 Hz, 2H), 7.35-7.29 (m, 1H), 4.86 (p, J=6.3 Hz, 1H), 4.42 (t, J=6.1 Hz, 2H), 4.04 (t, J=6.8 Hz, 2H), 2.90 (t, J=6.1 Hz, 2H), 2.44 (q, J=7.5 Hz, 4H), 2.24 (q, J=7.2 Hz, 4H), 1.59 (ddq, J=14.3, 7.2, 3.4, 2.3 Hz, 7H), 1.50 (d, J=6.2 Hz, 3H), 1.39-1.24 (m, 54H), 0.88 (td, J=6.9, 2.1 Hz, 10H). ¹³C NMR (101 MHZ, Chloroform-d) δ 173.66 (d, J=13.4 Hz), 147.37, 128.78, 127.95, 125.64, 120.56, 74.08, 64.45, 54.42, 54.29, 54.23, 49.02, 34.64, 34.23, 34.15, 31.91, 31.86, 29.60, 29.58, 29.53, 29.51, 29.33, 29.26, 29.24, 29.19, 28.65, 27.32, 27.15, 26.95, 26.91, 25.93, 25.32, 25.08, 24.86, 22.68, 22.66, 14.11. LC-MS: m/z 837.95 (M+H)⁺C₅₂H₉₂N₄O₄ (837.33).

### Example 3 Construction and Characterization of Ionizable Lipid Molecule B Library

It has been reported that the tail containing double bonds is associated with an increased tendency of bilayer lipids to form a non-bilayer phase, thereby facilitating the rupture of nanoparticles and effectively enhancing the release of nucleic acids. Therefore, Tail-3, a tail containing a cis double bond, is designed, and tail skeletons B22 (this tail skeleton is named B22 because the tail is Tail-2+Tail-2) and B23 (this tail skeleton is named B23 because the tail is Tail-2+Tail-3) are synthesized by modular combination with Tail-2; and the head structure selection is the same as that of A library, and the preparation method is shown in FIG. 2.

### Example 3.1 Construction and Characterization of Ionizable Lipid Molecule B22 Library

Specifically, the following steps are included:

### Step 1: Synthesis of B22 Tail Skeleton

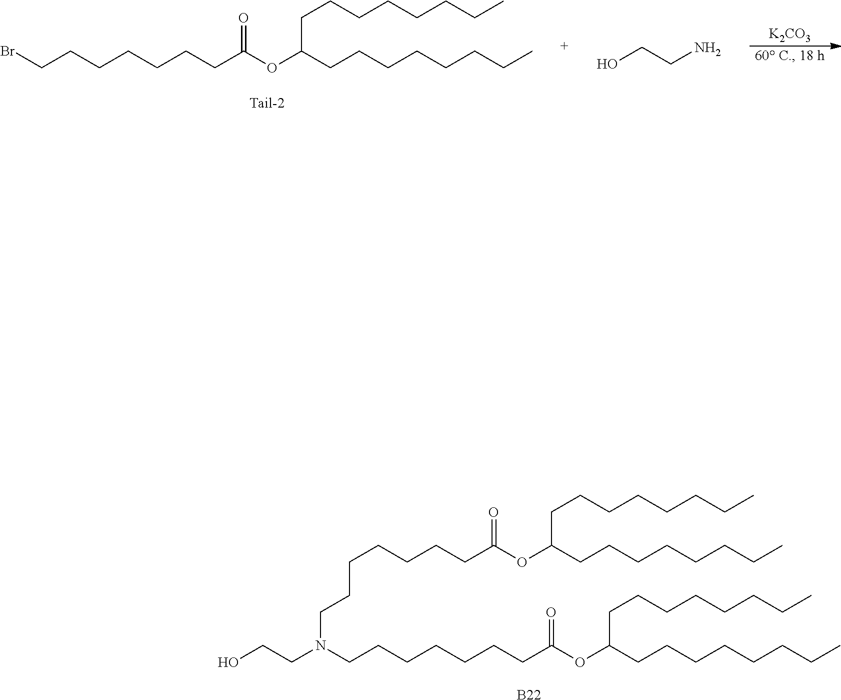

Tail-2 (8.0006 g, 0.0173 mol) was added to a round-bottom flask, followed by the addition of ethanolamine (30 mL), and the mixture was heated at 60° C. and reacted for 18 h with stirring. The reaction was monitored by TLC. After the reaction was completed, the reaction solution was diluted with EA and washed three times with saturated sodium chloride solution. The upper organic phase was collected and dried over anhydrous sodium sulfate for 30 minutes. The dried upper organic layer was filtered, the filtrate was subjected to rotary evaporation under reduced pressure, mixed with silica gel and then purified by silica gel column chromatography using an eluent of PE:EA=5:1. The product was collected to obtain a colorless oily liquid (3.9481 g) with a yield of 55.5%. An appropriate amount of the product was dissolved in deuterated chloroform CDCl₃ for NMR characterization: ¹H NMR (400 MHZ, Chloroform-d) δ 4.86 (p, J=6.3 Hz, 1H), 3.53 (t, J=5.4 Hz, 1H), 2.58 (t, J=5.4 Hz, 1H), 2.48-2.40 (m, 2H), 2.28 (t, J=7.5 Hz, 2H), 1.67-1.56 (m, 2H), 1.50 (p, J=5.4, 4.6 Hz, 4H), 1.43 (td, J=9.2, 8.3, 4.6 Hz, 2H), 1.36-1.20 (m, 31H), 0.920.84 (m, 6H). LC-MS: m/z 823.10 (M+H)⁺C₅₂H₁₀₃NO₅ (822.40).

### Step 2: Synthesis of N₃—B22 (Azide Tail Skeleton)

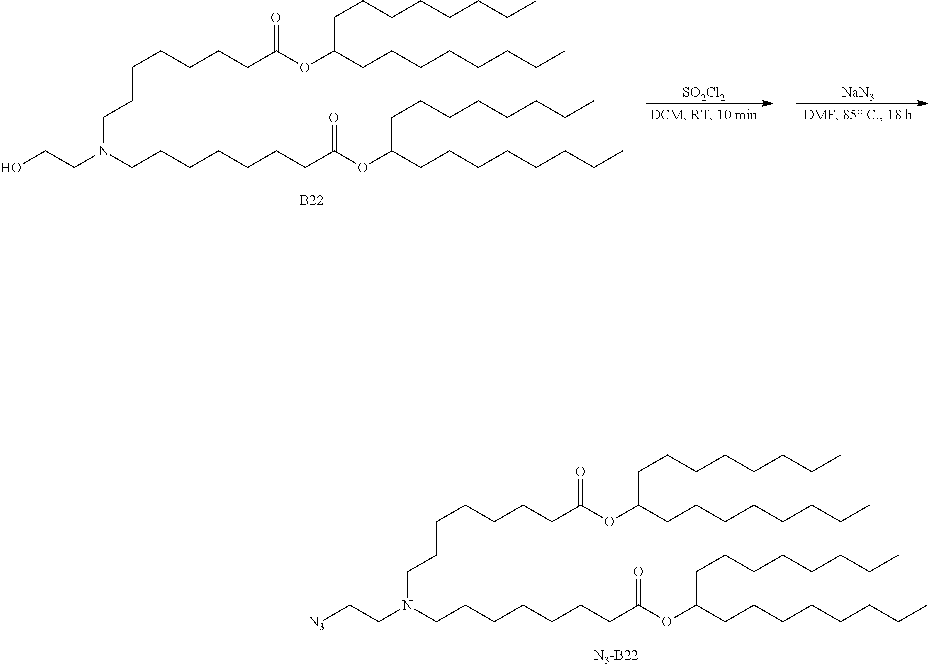

B22 (4.9200 g, 0.0060 mol) was added to a round-bottom flask and dissolved in DCM. SO₂Cl₂ (2.4235 g, 0.0180 mol) was added dropwise at room temperature with stirring. After the addition was completed, the mixture was reacted at room temperature for 10 min with stirring. The reaction was monitored by TLC. After the reaction was completed, the reaction was stopped, and the reaction solution was washed three times with a saturated sodium bicarbonate solution to remove the acid and make the reaction solution system alkaline. The lower organic layer was collected, dried over anhydrous sodium sulfate and filtered. The filtrate was subjected to rotary evaporation under reduced pressure to obtain crude product Cl—B22. The crude product Cl—B22 was directly dissolved in DMF, and an aqueous solution of NaN₃ (0.7782 g, 0.0120 mol) was added dropwise while stirring, and the mixture was stirred at room temperature for 10 min. The reaction was then transferred to an oil bath and stirred at 85° C. for 18 h. The reaction was monitored by TLC. After the reaction was completed, the reaction was stopped, DMF was removed by rotary evaporation under reduced pressure, and the residue was redissolved in EA and washed three times with saturated sodium chloride solution. The upper organic layer was collected and dried over anhydrous sodium sulfate for 30 minutes. The dried upper organic layer was filtered, the filtrate was subjected to rotary evaporation under reduced pressure, mixed with silica gel and then purified by silica gel column chromatography using an eluent of PE:EA=50:1. The product was collected to obtain a colorless oily liquid (4.0601 g) with a yield of 80.1%. An appropriate amount of the product was dissolved in deuterated chloroform CDCl₃ for NMR characterization: ¹H NMR (400 MHZ, Chloroform-d) δ 4.86 (p, J=6.3 Hz, 2H), 3.25 (t, J=6.2 Hz, 2H), 2.63 (t, J=6.3 Hz, 2H), 2.52-2.37 (m, 4H), 2.28 (t, J=7.5 Hz, 4H), 1.61 (d, J=8.9 Hz, 6H), 1.50 (d, J=6.2 Hz, 7H), 1.46-1.38 (m, 4H), 1.37-1.12 (m, 59H), 0.88 (t, J=6.8 Hz, 12H). LC-MS: m/z 848.10 (M+H)⁺C₅₂H₁₀₂N₄₀₄ (847.41).

### Step 3: Preparation of LipidB22-X (CuAAC method)

The synthesis steps of LipidB22-1 to LipidB22-15 are as follows:

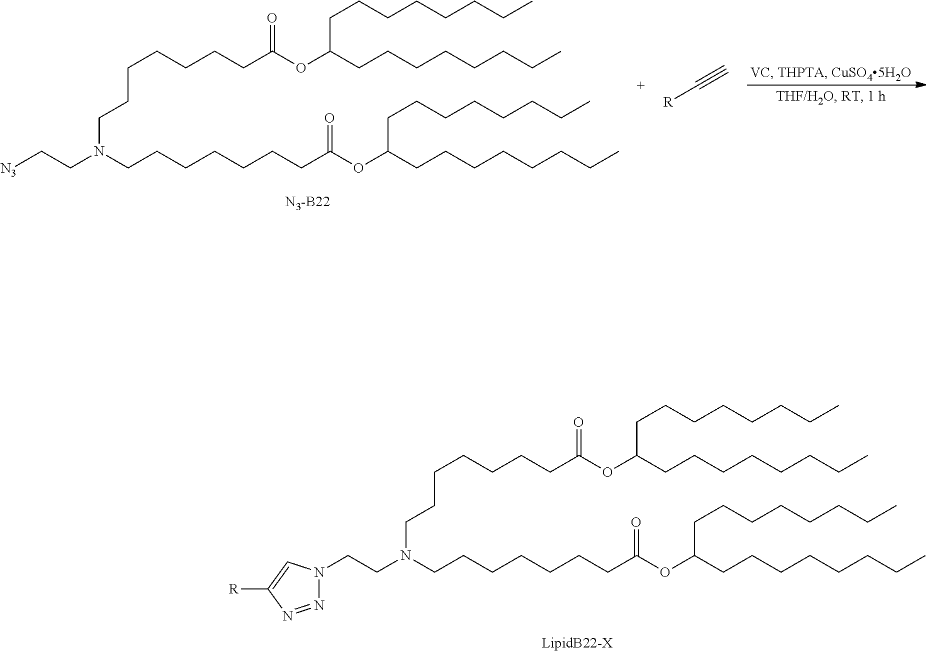

N₃-B22-X, VC, THPTA, CuSO₄ and a head small molecule of a terminal alkyne were weighed based on the equivalent weight in Table 2, dissolved in the corresponding solvents, and added into a flask in the order of N₃-B22-1, VC, THPTA, CuSO₄ and R—X. The solvent system was adjusted to THF:H₂O:DMSO=4:1:0.05. The mixture was reacted at room temperature for 1 hour with stirring. The reaction was monitored by TLC. After the reaction was completed, the reaction solution was evaporated to dryness under reduced pressure, redissolved in EA, and washed 5 times with saturated sodium chloride solution to obtain pure product LipidB22-X without further purification by silica gel column chromatography.

TABLE 2
Feed ratio and usage amount details for synthesis of LipidB22-X

| | N₃-B22 | VC | THPTA | CuSO₄•5H₂O |
|---|---|---|---|---|
| M | 847.41 | 176.13 | 434.25 | 249.68 |
| eq | 1 | 1 | 0.1 | 1 |
| m/mg | 100.0 | 20.8 | 5.1 | 29.5 |

| | R-1 | R-2 | R-3 | R-4 | R-5 |
|---|---|---|---|---|---|
| M | 70.09 | 84.12 | 84.12 | 84.12 | 83.13 |
| eq | 1.2 | | | | |
| m/mg | 9.9 | 11.9 | 11.9 | 11.9 | 11.8 |

| | R-6 | R-7 | R-8 | R-9 | R-10 |
|---|---|---|---|---|---|
| M | 111.19 | 109.17 | 138.21 | 69.11 | 68.12 |
| eq | 1.2 | | | | |
| m/mg | 15.7 | 15.5 | 19.6 | 9.8 | 9.65 |

| | R-11 | R-12 | R-13 | R-14 | R-15 |
|---|---|---|---|---|---|
| M | 82.15 | 66.10 | 140.18 | 128.17 | 102.14 |
| eq | 1.2 | | | | |
| m/mg | 11.6 | 9.4 | 19.9 | 18.1 | 14.5 |

The obtained products and characterizations thereof are as follows:

LipidB22-1: ¹H NMR (400 MHZ, Chloroform-d) δ 7.49 (d, J=24.5 Hz, 1H), 4.79 (p, J=6.2 Hz, 2H), 4.36 (d, J=11.0 Hz, 2H), 4.09-3.57 (m, 2H), 2.89 (d, J=12.0 Hz, 4H), 2.60-2.32 (m, 4H), 2.21 (t, J=7.5 Hz, 4H), 1.54 (d, J=14.7 Hz, 4H), 1.43 (q, J=6.0 Hz, 8H), 1.33 (s, 3H), 1.20 (d, J=7.6 Hz, 62H), 0.89-0.70 (m, 12H). ¹³C NMR (101 MHZ, Chloroform-d) δ 172.62, 73.17, 53.23, 33.63, 33.12, 30.85, 28.52, 28.49, 28.22, 28.17, 28.13, 26.14, 24.30, 24.03, 21.65, 13.09. LC-MS: m/z 918.45 (M+H)⁺C₅₆H₁₀₈N₄₀₅ (917.50).

LipidB22-2: ¹H NMR (400 MHZ, Chloroform-d) δ 7.44 (s, 1H), 4.86 (p, J=6.3 Hz, 2H), 4.46-4.29 (m, 2H), 3.71 (t, J=6.1 Hz, 2H), 2.88 (s, 1H), 2.83 (t, J=7.3 Hz, 2H), 2.51-2.37 (m, 4H), 2.28 (t, J=7.5 Hz, 4H), 1.93 (p, J=6.6 Hz, 2H), 1.61 (t, J=7.3 Hz, 4H), 1.50 (d, J=6.1 Hz, 8H), 1.27 (d, J=9.2 Hz, 67H), 0.88 (t, J=6.8 Hz, 12H). ¹³C NMR (101 MHZ, Chloroform-d) δ 173.67, 74.19, 61.94, 54.37, 34.68, 34.14, 32.08, 31.86, 29.54, 29.51, 29.24, 29.19, 27.25, 25.32, 25.09, 22.66, 22.18, 14.11. LC-MS: m/z 932.20 (M+H)⁺C₅₇H₁₁₀N₄₀₅ (931.53).

LipidB22-3: ¹H NMR (400 MHZ, Chloroform-d) δ 7.61 (s, 1H), 4.85 (h, J=6.4 Hz, 3H), 4.37 (d, J=16.7 Hz, 1H), 2.88 (s, 2H), 2.50-2.37 (m, 3H), 2.27 (t, J=7.5 Hz, 4H), 1.90 (ddt, J=21.4, 14.0, 7.3 Hz, 2H), 1.62 (q, J=7.1 Hz, 5H), 1.27 (d, J=8.4 Hz, 67H), 0.99 (t, J=7.4 Hz, 3H), 0.92-0.83 (m, 12H). ¹³C NMR (101 MHZ, Chloroform-d) δ 173.67, 74.18, 54.34, 34.67, 34.14, 31.87, 29.54, 29.51, 29.24, 29.22, 29.17, 27.23, 25.32, 25.06, 22.67, 14.11, 9.75. LC-MS: m/z 932.20 (M+H)⁺C₅₇H₁₁₀N₄₀₅ (931.53).

LipidB22-4: ¹H NMR (400 MHZ, Chloroform-d) δ 7.51 (s, 1H), 4.86 (p, J=6.3 Hz, 2H), 4.44-4.31 (m, 2H), 4.21-4.07 (m, 1H), 2.97-2.84 (m, 2H), 2.76 (dd, J=14.9, 8.1 Hz, 1H), 2.50-2.39 (m, 3H), 2.28 (t, J=7.5 Hz, 4H), 1.61 (p, J=7.3 Hz, 4H), 1.57-1.46 (m, 8H), 1.27 (d, J=8.4 Hz, 69H), 0.88 (t, J=6.7 Hz, 12H). ¹³C NMR (101 MHZ, Chloroform-d) δ 173.64, 74.17, 54.36, 34.67, 34.14, 31.87, 29.54, 29.51, 29.24, 29.18, 27.23, 25.32, 25.07, 22.67, 14.11. LC-MS: m/z 932.20 (M+H)⁺C₅₇H₁₁₀N₄₀₅ (931.53).

LipidB22-5: ¹H NMR (400 MHZ, Chloroform-d) δ 7.66 (s, 1H), 4.86 (p, J=6.2 Hz, 2H), 4.64-4.21 (m, 1H), 3.58 (q, J=47.2 Hz, 1H), 3.15-2.66 (m, 2H), 2.58-2.38 (m, 4H), 2.28 (t, J=7.4 Hz, 6H), 1.62 (q, J=7.1 Hz, 4H), 1.50 (d, J=6.1 Hz, 8H), 1.28 (d, J=15.8 Hz, 67H), 0.88 (t, J=6.6 Hz, 13H). ¹³C NMR (101 MHZ, Chloroform-d) δ 173.58, 74.09, 54.39, 34.67, 34.13, 31.85, 29.52, 29.49, 29.27, 29.22, 27.29, 27.17, 25.31, 25.10, 22.65, 14.09. LC-MS: m/z 931.35 (M+H)⁺C₅₇H₁₁₁N₅₀₄ (930.55).

LipidB22-6: ¹H NMR (400 MHZ, Chloroform-d) δ 7.63 (s, 1H), 4.87 (h, J=6.7 Hz, 2H), 4.36 (t, J=6.3 Hz, 2H), 3.85 (s, 2H), 2.86 (t, J=6.4 Hz, 2H), 2.60 (d, J=7.3 Hz, 3H), 2.42 (dd, J=8.6, 6.2 Hz, 4H), 2.27 (t, J=7.5 Hz, 4H), 1.61 (p, J=7.7 Hz, 4H), 1.50 (d, J=6.2 Hz, 8H), 1.27 (d, J=9.2 Hz, 64H), 1.12 (t, J=7.0 Hz, 6H), 0.87 (t, J=6.8 Hz, 12H). ¹³C NMR (101 MHZ, Chloroform-d) δ 173.58, 74.10, 54.41, 54.18, 34.66, 34.14, 31.85, 29.52, 29.49, 29.27, 29.22, 29.21, 27.28, 27.21, 25.31, 25.10, 22.65, 14.09. LC-MS: m/z 959.25 (M+H)⁺C₅₉H₁₁₅N₅₀₄ (958.60).

LipidB22-7: ¹H NMR (400 MHZ, Chloroform-d) δ 7.68 (s, 1H), 4.86 (p, J=6.3 Hz, 2H), 4.37 (d, J=7.4 Hz, 2H), 3.86 (s, 1H), 2.86 (t, J=6.4 Hz, 2H), 2.65 (d, J=24.2 Hz, 3H), 2.48-2.36 (m, 4H), 2.27 (t, J=7.5 Hz, 4H), 1.83 (s, 3H), 1.61 (p, J=7.4 Hz, 4H), 1.50 (d, J=6.2 Hz, 6H), 1.27 (d, J=8.3 Hz, 68H), 0.95-0.80 (m, 12H). ¹³C NMR (101 MHZ, Chloroform-d) δ 173.58, 74.09, 54.44, 34.67, 34.14, 31.85, 29.52, 29.49, 29.27, 29.22, 29.21, 27.28, 27.21, 25.31, 25.10, 22.65, 14.09. LC-MS: m/z 957.45 (M+H)⁺C₅₉H₁₁₃N₅₀₄ (956.58).

LipidB22-8: ¹H NMR (400 MHZ, Chloroform-d) δ 7.55 (s, 1H), 4.79 (p, J=6.3 Hz, 2H), 4.30 (t, J=6.4 Hz, 2H), 3.67 (s, 2H), 2.80 (t, J=6.4 Hz, 2H), 2.64 (s, 6H), 2.43-2.32 (m, 6H), 2.21 (t, J=7.5 Hz, 4H), 1.54 (p, J=7.0 Hz, 4H), 1.43 (q, J=6.1 Hz, 8H), 1.34-1.00 (m, 68H), 0.87-0.76 (m, 12H). ¹³C NMR (101 MHZ, Chloroform-d) δ 173.58, 74.13, 54.40, 34.67, 34.14, 31.86, 29.53, 29.50, 29.27, 29.23, 29.21, 27.28, 27.09, 25.31, 25.10, 22.66, 14.10. LC-MS: m/z 986.25 (M+H)⁺C₆₀H₁₁₆N₆₀₄ (985.63).

LipidB22-9: ¹H NMR (400 MHZ, Chloroform-d) δ 4.86 (p, J=6.3 Hz, 2H), 4.47-4.27 (m, 1H), 2.92-2.80 (m, 1H), 2.56-2.37 (m, 3H), 2.27 (t, J=7.4 Hz, 4H), 1.62 (q, J=7.0 Hz, 4H), 1.50 (d, J=6.1 Hz, 9H), 1.26 (s, 69H), 0.87 (t, J=6.7 Hz, 16H). ¹³C NMR (101 MHZ, Chloroform-d) δ 173.61, 74.12, 34.70, 34.67, 34.14, 31.86, 29.69, 29.53, 29.50, 29.30, 29.23, 27.28, 27.07, 25.32, 25.12, 22.66, 14.10. LC-MS: m/z 917.35 (M+H)⁺C₅₆H₁₀₉N₅₀₄ (916.52).

LipidB22-10: ¹H NMR (400 MHZ, Chloroform-d) δ 7.31 (s, 1H), 4.79 (p, J=6.3 Hz, 2H), 4.27 (t, J=6.3 Hz, 2H), 2.78 (t, J=6.3 Hz, 2H), 2.61 (t, J=7.6 Hz, 2H), 2.35 (t, J=7.4 Hz, 4H), 2.20 (t, J=7.5 Hz, 4H), 1.63 (dt, J=15.0, 7.5 Hz, 2H), 1.54 (d, J=14.8 Hz, 4H), 1.43 (q, J=6.0 Hz, 8H), 1.20 (d, J=8.3 Hz, 67H), 0.90 (t, J=7.3 Hz, 3H), 0.85-0.76 (m, 12H). ¹³C NMR (101 MHZ, Chloroform-d) δ 173.59, 121.42, 74.11, 54.44, 54.27, 48.73, 34.67, 34.14, 31.86, 29.53, 29.50, 29.26, 29.23, 29.20, 27.71, 27.28, 27.16, 25.31, 25.10, 22.79, 22.66, 14.10. LC-MS: m/z 916.30 (M+H)⁺C₅₇H₁₁₀N₄₀₄ (915.53).

LipidB22-11: ¹H NMR (400 MHZ, Chloroform-d) δ 7.41 (s, 1H), 4.86 (p, J=6.2 Hz, 2H), 4.41 (q, J=6.1 Hz, 1H), 2.89 (dd, J=14.6, 8.5 Hz, 2H), 2.58 (d, J=6.9 Hz, 1H), 2.44 (dt, J=15.2, 7.4 Hz, 3H), 2.27 (t, J=7.5 Hz, 4H), 1.60 (dp, J=11.5, 3.9 Hz, 5H), 1.50 (d, J=6.1 Hz, 8H), 1.44-1.14 (m, 66H), 1.13-0.70 (m, 18H). ¹³C NMR (101 MHZ, Chloroform-d) δ 173.61, 173.59, 77.25, 74.14, 54.32, 34.67, 34.16, 31.87, 29.55, 29.51, 29.25, 29.23, 29.19, 29.17, 27.25, 25.33, 25.10, 22.67, 22.32, 18.53, 14.11.

LipidB22-12: ¹H NMR (400 MHZ, Chloroform-d) δ 7.32 (s, 1H), 4.86 (p, J=6.2 Hz, 2H), 4.30 (t, J=6.3 Hz, 2H), 2.82 (t, J=6.3 Hz, 2H), 2.41 (t, J=7.4 Hz, 3H), 2.28 (t, J=7.5 Hz, 4H), 1.94 (tt, J=8.4, 5.0 Hz, 1H), 1.61 (d, J=14.6 Hz, 4H), 1.50 (q, J=6.0 Hz, 8H), 1.27 (d, J=8.5 Hz, 67H), 0.95-0.79 (m, 16H). ¹³C NMR (101 MHZ, Chloroform-d) § 173.60, 120.41, 74.11, 54.45, 54.27, 48.79, 34.68, 34.15, 31.86, 29.53, 29.50, 29.26, 29.23, 29.20, 27.29, 27.18, 25.32, 25.11, 22.66, 14.10, 7.62, 6.69. LC-MS: m/z 914.55 (M+H)⁺C₅₇H₁₀₈N₄₀₄ (913.52).

LipidB22-13: ¹H NMR (400 MHz, Chloroform-d) δ 7.65 (s, 1H), 4.91-4.84 (m, 3H), 4.79 (dt, J=30.9, 3.5 Hz, 2H), 4.67 (s, 1H), 4.37 (t, J=6.4 Hz, 2H), 4.34-4.17 (m, 2H), 3.88 (dddd, J=29.8, 11.5, 8.3, 3.0 Hz, 2H), 3.55 (dddd, J=15.4, 8.4, 3.9, 1.6 Hz, 2H), 2.86 (t, J=6.4 Hz, 2H), 2.46-2.38 (m, 4H), 2.27 (t, J=7.5 Hz, 4H), 1.92-1.70 (m, 4H), 1.65-1.47 (m, 20H), 1.27 (d, J=8.4 Hz, 67H), 0.92-0.83 (m, 12H). ¹³C NMR (101 MHZ, Chloroform-d) δ 173.57, 123.26, 98.03, 96.82, 74.09, 73.96, 62.25, 61.97, 60.52, 54.42, 54.19, 53.97, 48.84, 34.66, 34.14, 31.85, 30.48, 30.20, 29.52, 29.49, 29.25, 29.22, 29.19, 27.28, 27.14, 25.41, 25.31, 25.10, 22.65, 19.38, 18.99, 14.09. LC-MS: m/z 988.35 (M+H)⁺C₆₀H₁₁₄N₄₀₆ (987.59).

LipidB22-14: ¹H NMR (400 MHZ, Chloroform-d) δ 7.70 (s, 1H), 5.71 (s, 1H), 4.86 (p, J=6.3 Hz, 2H), 4.37 (q, J=7.4, 6.3 Hz, 1H), 3.77-3.54 (m, 3H), 2.86 (t, J=6.4 Hz, 2H), 2.42 (dd, J=8.7, 6.1 Hz, 3H), 2.27 (t, J=7.5 Hz, 4H), 1.61 (p, J=7.4 Hz, 4H), 1.50 (t, J=6.0 Hz, 8H), 1.27 (d, J=8.3 Hz, 71H), 0.88 (t, J=6.7 Hz, 13H). ¹³C NMR (101 MHZ, Chloroform-d) δ 173.58, 74.10, 61.52, 54.42, 34.67, 34.14, 31.86, 29.52, 29.50, 29.26, 29.23, 29.20, 27.27, 27.19, 25.31, 25.11, 22.65, 15.17, 14.09. LC-MS: m/z 976.20 (M+H)⁺C₅₉H₁₁₄N₄₀₆ (975.58).

LipidB22-15: ¹H NMR (400 MHZ, Chloroform-d) δ 7.82 (s, 1H), 7.76 (dd, J=7.3, 1.7 Hz, 2H), 7.34 (dd, J=8.4, 6.9 Hz, 2H), 7.28-7.19 (m, 1H), 4.79 (p, J=6.3 Hz, 2H), 4.34 (t, J=6.1 Hz, 2H), 2.82 (t, J=6.1 Hz, 2H), 2.36 (t, J=7.4 Hz, 4H), 2.16 (t, J=7.5 Hz, 4H), 1.51 (q, J=7.3 Hz, 4H), 1.42 (t, J=6.1 Hz, 7H), 1.18 (s, 69H), 0.80 (t, J=6.8 Hz, 12H). ¹³C NMR (101 MHZ, Chloroform-d) δ 173.60, 128.78, 127.95, 125.64, 120.56, 74.09, 54.46, 54.31, 49.07, 34.65, 34.15, 31.86, 29.54, 29.51, 29.28, 29.24, 29.20, 27.35, 27.22, 25.32, 25.09, 22.66, 14.10. LC-MS: m/z 950.10 (M+H)⁺C₆₀H₁₁₈N₄₀₄ (949.55).

### Example 3.2 Construction and Characterization of Ionizable Lipid Molecule B23 Library

Specifically, the following steps are included:

### Step 1: Synthesis of Tail-3

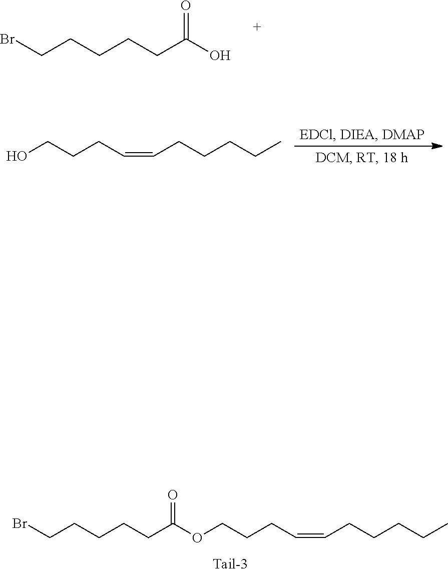 [SMILES: O=C(O)CCCCCBr ; "EDCl, DIEA, DMAP" ; CCCCC/C=C\CCCO ; "DCM, RT, 18 h" ; CCCCC/C=C\CCCOC(=O)CCCCCBr]

6-Bromohexanoic acid (10.0055 g, 0.0513 mol) was added to a round-bottom flask and dissolved in DCM. Cis-4-decen-1-ol (8.8174 g, 0.0564 mol), EDCI (14.7497 g, 0.0769 mol), DIEA (16.5775 g, 0.1283 mol) and DMAP (0.9402 g, 0.0077 mol) were added. The mixture was reacted at room temperature for 18 h with stirring. The reaction was monitored by TLC. After the reaction was completed, the solvent was concentrated by evaporation, redissolved in EA, and washed three times with 3% KHSO4 solution. The upper organic phase was collected and dried over anhydrous sodium sulfate for 30 minutes. The organic phase was filtered, concentrated by evaporation, mixed with silica gel, and purified by silica gel column chromatography in an elution system of PE:EA=100:1. The product was collected to obtain a colorless oily liquid (8.8852 g) with a yield of 52.0%. An appropriate amount of the product was dissolved in deuterated chloroform CDCl for NMR characterization: ¹H NMR (400 MHZ, Chloroform-d) δ 5.57-5.11 (m, 2H), 4.07 (t, J=6.6 Hz, 2H), 3.54 (t, J=6.7 Hz, 2H), 2.33 (t, J=7.4 Hz, 2H), 2.06 (dq, J=36.6, 7.1 Hz, 4H), 1.92-1.62 (m, 6H), 1.53-1.43 (m, 2H), 1.42-1.16 (m, 6H), 0.89 (t, J=6.9 Hz, 3H).

### Step 2: Synthesis of B23 Tail Skeleton

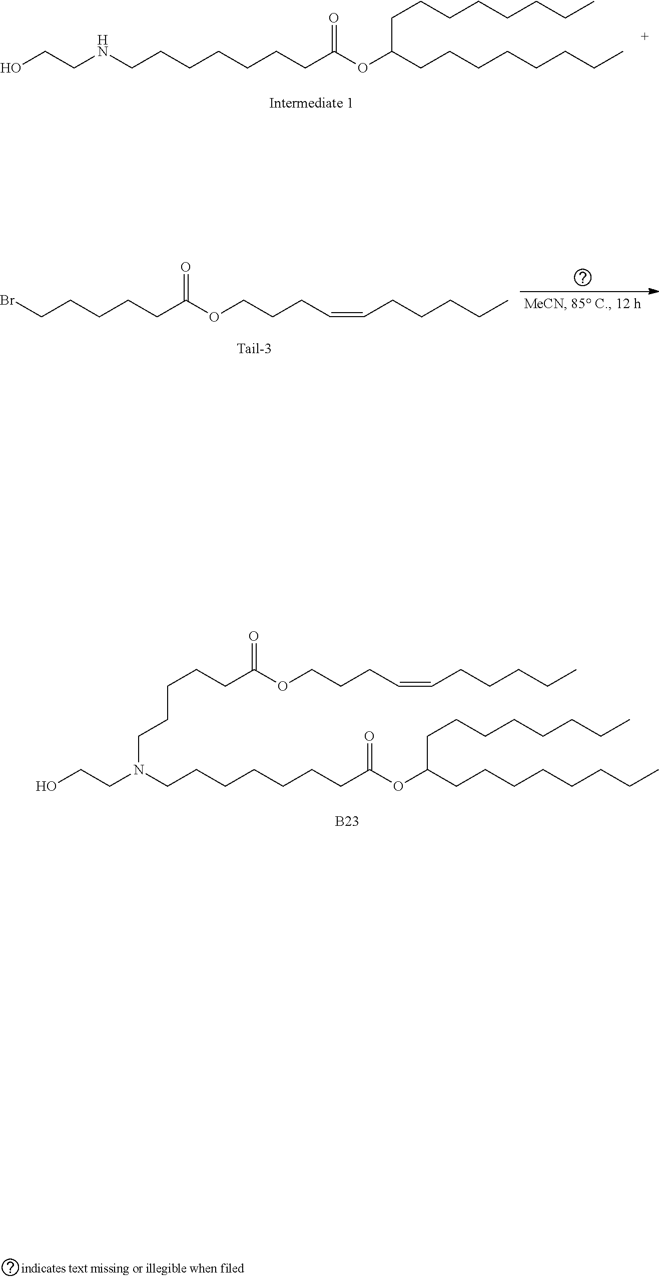

The intermediate 1 (5.0094 g, 0.0113 mol) was added to a round-bottom flask and dissolved in MeCN, and Tail-1 (4.1578 g, 0.0125 mol), K₂CO₃ (6.2469 g, 0.0452 mol) and KI (0.4689 g, 0.0028 mol) were added. The mixture was reacted at 85° C. for 12 h with stirring. The reaction was monitored by TLC. After the reaction was completed, MeCN was removed by rotary evaporation, and the residue was redissolved in EA and washed three times with saturated sodium chloride solution. The upper organic layer was collected and dried over anhydrous sodium sulfate for 30 minutes. The upper organic layer was filtered, the filtrate was subjected to rotary evaporation under reduced pressure, mixed with silica gel and then purified by silica gel column chromatography using an eluent of EA:MeOH=10:1. The product was collected to obtain a colorless oily liquid (3.5532 g) with a yield of 45.3%. An appropriate amount of the product was dissolved in deuterated chloroform CDCl₃ for NMR characterization: ¹H NMR (400 MHZ, Chloroform-d) δ 5.47-5.26 (m, 2H), 4.86 (p, J=6.3 Hz, 1H), 4.07 (t, J=6.7 Hz, 2H), 3.52 (t, J=5.4 Hz, 2H), 2.57 (t, J=5.4 Hz, 2H), 2.44 (dt, J=7.8, 5.5 Hz, 4H), 2.29 (dt, J=11.3, 7.5 Hz, 4H), 2.10 (q, J=7.3 Hz, 2H), 2.01 (q, J=6.8 Hz, 3H), 1.73-1.57 (m, 7H), 1.56-1.39 (m, 9H), 1.37-1.19 (m, 40H), 0.88 (td, J=6.8, 4.3 Hz, 9H). LC-MS: m/z 694.80 (M+H)⁺C₄₃H₈₃NO₅ (694.14).

### Step 3: Synthesis of N₃—B23 (Azide Tail Skeleton)

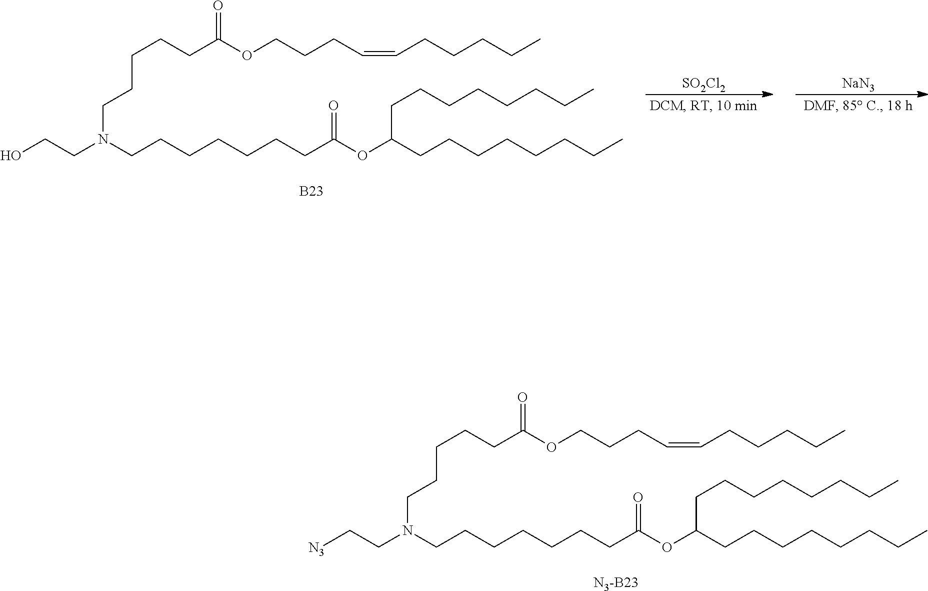

B23 (3.5000 g, 0.0050 mol) was added to a round-bottom flask and dissolved in DCM. SO₂Cl₂ (2.0416 g, 0.0151 mol) was added dropwise at room temperature with stirring. After the addition was completed, the mixture was reacted at room temperature for 10 min with stirring. The reaction was monitored by TLC. After the reaction was completed, the reaction was stopped, and the reaction solution was washed three times with a saturated sodium bicarbonate solution to remove the acid and make the reaction solution system alkaline. The lower organic layer was collected, dried over anhydrous sodium sulfate and filtered. The filtrate was subjected to rotary evaporation under reduced pressure to obtain crude product Cl—B23. The crude product Cl—B23 was directly dissolved in DMF, and an aqueous solution of NaN₃ (0.6556 g, 0.0101 mol) was added dropwise while stirring, and the mixture was stirred at room temperature for 10 min. The reaction was then transferred to an oil bath and stirred at 85° C. for 18 h. The reaction was monitored by TLC. After the reaction was completed, the reaction was stopped, DMF was removed by rotary evaporation under reduced pressure, and the residue was redissolved in EA and washed three times with saturated sodium chloride solution. The upper organic layer was collected and dried over anhydrous sodium sulfate for 30 minutes. The dried upper organic layer was filtered, the filtrate was subjected to rotary evaporation under reduced pressure, mixed with silica gel and then purified by silica gel column chromatography using an eluent of PE:EA=50:1. The product was collected to obtain a colorless oily liquid (2.9773 g) with a yield of 82.8%. An appropriate amount of the product was dissolved in deuterated chloroform CDCl₃ for NMR characterization: ¹H NMR (400 MHz, Chloroform-d) δ 4.86 (p, J=6.3 Hz, 1H), 4.19-3.93 (m, 4H), 3.25 (t, J=6.3 Hz, 2H), 2.64 (t, J=6.2 Hz, 2H), 2.51-2.36 (m, 4H), 2.30 (dt, J=15.4, 7.5 Hz, 4H), 2.09-1.69 (m, 6H), 1.69-1.58 (m, 5H), 1.47 (dt, J=20.8, 6.0 Hz, 9H), 1.38-1.17 (m, 38H), 0.89 (dt, J=11.5, 7.0 Hz, 9H). LC-MS: m/z 719.80 (M+H)⁺C₅₂H₈₂N₄O₄ (719.15).

### Step 4: Preparation of LipidB23-X (CuAAC method)

The synthesis steps of LipidB23-1 to LipidB23-15 are as follows:

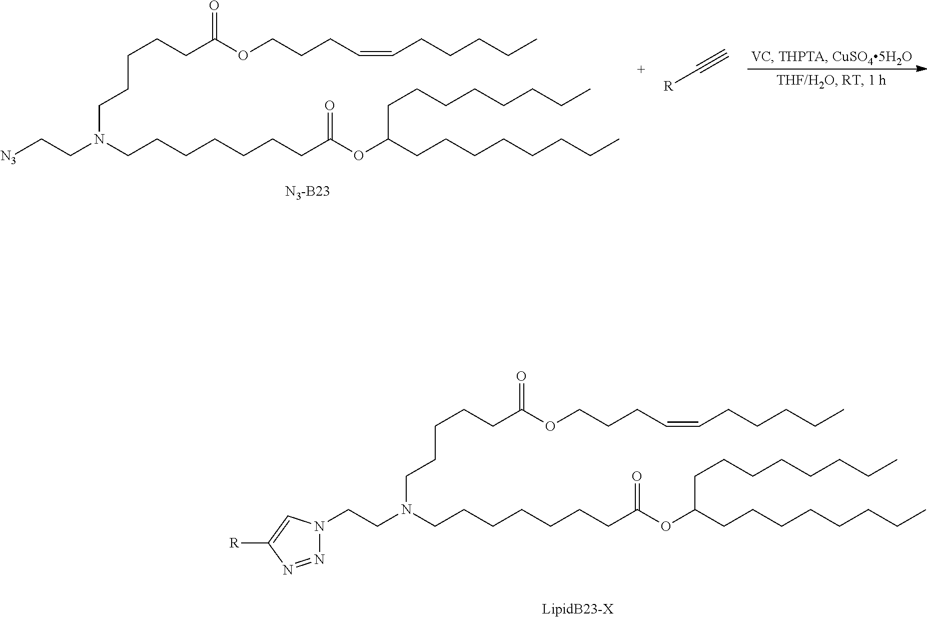

N₃-B23-1, VC, THPTA, CuSO₄ and a head small molecule of a terminal alkyne were weighed based on the equivalent weight in Table 3, dissolved in the corresponding solvents, and added into a flask in the order of N₃-B23-1, VC, THPTA, CuSO₄ and R—X. The solvent system was adjusted to THF:H₂O:DMSO=4:1:0.05. The mixture was reacted at room temperature for 1 hour with stirring. The reaction was monitored by TLC. After the reaction was completed, the reaction solution was evaporated to dryness under reduced pressure, redissolved in EA, and washed 5 times with saturated sodium chloride solution to obtain pure product LipidB23-X without further purification by silica gel column chromatography.

TABLE 3
Feed ratio and usage amount details for synthesis of LipidB23-X

| | N₃-B23 | VC | THPTA | CuSO₄•5H₂O |
|---|---|---|---|---|
| M | 719.15 | 176.13 | 434.25 | 249.68 |
| eq | 1 | 1 | 0.1 | 1 |
| m/mg | 100.0 | 24.5 | 6.0 | 34.7 |

| | R-1 | R-2 | R-3 | R-4 | R-5 |
|---|---|---|---|---|---|
| M | 70.09 | 84.12 | 84.12 | 84.12 | 83.13 |
| eq | 1.2 | | | | |
| m/mg | 11.7 | 14.0 | 14.0 | 14.0 | 13.9 |

| | R-6 | R-7 | R-8 | R-9 | R-10 |
|---|---|---|---|---|---|
| M | 111.19 | 109.17 | 138.21 | 69.11 | 68.12 |
| eq | 1.2 | | | | |
| m/mg | 18.6 | 18.2 | 23.1 | 11.5 | 11.4 |

| | R-11 | R-12 | R-13 | R-14 | R-15 |
|---|---|---|---|---|---|
| M | 82.15 | 66.10 | 140.18 | 128.17 | 102.14 |
| eq | 1.2 | | | | |
| m/mg | 13.7 | 11.0 | 23.4 | 21.4 | 17.0 |

The obtained products and characterizations thereof are as follows:

LipidB23-1: ¹H NMR (400 MHZ, Chloroform-d) δ 7.53 (s, 1H), 4.93-4.80 (m, 1H), 4.36 (t, J=5.8 Hz, 2H), 4.18-4.01 (m, 4H), 3.94 (s, 2H), 3.01-2.77 (m, 5H), 2.43 (t, J=6.9 Hz, 4H), 2.29 (q, J=7.3 Hz, 4H), 2.03-1.70 (m, 6H), 1.68-1.45 (m, 10H), 1.42-1.14 (m, 44H), 0.96-0.79 (m, 9H). ¹³C NMR (101 MHZ, Chloroform-d) δ 173.65, 74.16, 65.45, 64.90, 63.43, 61.68, 54.35, 54.13, 34.65, 34.29, 34.14 (d, J=5.0 Hz), 31.84, 31.13 (d, J=2.7 Hz), 29.50 (d, J=3.1 Hz), 29.19 (d, J=5.0 Hz), 27.22, 26.85, 26.34, 26.06, 25.30, 25.06, 24.81, 22.64, 22.43, 14.09, 13.95. LC-MS: m/z 932.20 (M+H)⁺C₅₇H₁₁₀N₄₀₅ (931.53). LC-MS: m/z 789.80 (M+H)⁺C₄₇H₈₈N₄O₅ (789.24).

LipidB23-2: ¹H NMR (400 MHZ, Chloroform-d) δ 7.43 (s, 1H), 4.86 (p, J=6.2 Hz, 1H), 4.35 (t, J=6.1 Hz, 2H), 4.14-4.03 (m, 3H), 3.70 (t, J=6.1 Hz, 2H), 2.84 (dt, J=14.5, 6.7 Hz, 5H), 2.43 (t, J=7.2 Hz, 4H), 2.29 (q, J=7.5 Hz, 4H), 2.03-1.70 (m, 8H), 1.67-1.45 (m, 10H), 1.40-1.17 (m, 46H), 0.88 (h, J=7.1 Hz, 10H). ¹³C NMR (101 MHZ, Chloroform-d) δ 173.65, 74.15, 65.44, 64.90, 63.44, 61.75, 54.36, 54.17, 54.15, 48.76, 34.65, 34.29, 34.17, 34.11, 32.11, 31.84, 31.14, 31.12, 29.51, 29.48, 29.21, 29.17, 27.24, 27.03, 26.85, 26.33, 26.05, 25.29, 25.07, 24.81, 22.64, 22.43, 22.08, 14.09, 13.95. LC-MS: m/z 803.95 (M+H)⁺C₄₈H₉₀N₄O₅ (803.27).

LipidB23-3: ¹H NMR (400 MHZ, Chloroform-d) δ 7.51 (s, 1H), 4.86 (p, J=6.3 Hz, 1H), 4.36 (t, J=6.2 Hz, 2H), 4.20-4.02 (m, 5H), 2.94-2.72 (m, 4H), 2.50-2.39 (m, 4H), 2.29 (q, J=7.3 Hz, 5H), 2.04-1.69 (m, 6H), 1.67-1.47 (m, 10H), 1.42-1.15 (m, 52H), 0.89 (dt, J=11.6, 6.8 Hz, 9H). ¹³C NMR (101 MHZ, Chloroform-d) δ 173.61, 74.13, 65.44, 64.89, 63.42, 54.34, 54.14, 48.79, 34.83, 34.64, 34.28, 34.12, 31.84, 31.14, 31.12, 29.51, 29.48, 29.21, 29.16, 27.22, 27.04, 26.85, 26.33, 26.06, 25.29, 25.06, 24.80, 22.86, 22.64, 22.43, 14.09, 13.95. LC-MS: m/z 803.95 (M+H)⁺C₄₈H₉₀N₄O₅ (803.27).

LipidB23-4: ¹H NMR (400 MHZ, Chloroform-d) δ 7.61 (s, 1H), 4.85 (dq, J=13.6, 6.5 Hz, 2H), 4.38 (t, J=6.1 Hz, 2H), 4.18-4.02 (m, 4H), 2.89 (t, J=6.2 Hz, 2H), 2.44 (t, J=7.4 Hz, 4H), 2.28 (q, J=7.1 Hz, 5H), 2.03-1.68 (m, 9H), 1.66-1.46 (m, 10H), 1.42-1.18 (m, 46H), 1.01 (dt, J=14.7, 7.4 Hz, 4H), 0.89 (dt, J=11.5, 6.9 Hz, 9H). ¹³C NMR (101 MHZ, Chloroform-d) δ 74.15, 68.39, 65.44, 64.90, 63.46, 54.30, 54.08, 34.64, 34.29, 34.15, 34.12, 31.84, 31.15, 31.11, 30.40, 29.51, 29.48, 29.21, 29.15, 27.21, 26.82, 26.34, 26.05, 25.29, 25.05, 24.78, 22.64, 22.43, 14.09, 13.95, 9.76. LC-MS: m/z 803.95 (M+H)⁺C₄₈H₉₀N₄O₅ (803.27).

LipidB23-5: ¹H NMR (400 MHz, Chloroform-d) δ 4.86 (p, J=6.2 Hz, 1H), 4.10 (dd, J=15.4, 8.3 Hz, 5H), 2.63-2.38 (m, 5H), 2.29 (q, J=7.1, 6.5 Hz, 7H), 2.00-1.69 (m, 7H), 1.69-1.47 (m, 11H), 1.43-1.17 (m, 49H), 0.89 (dt, J=12.8, 6.6 Hz, 11H). ¹³C NMR (101 MHZ, Chloroform-d) & 173.65, 74.16, 65.45, 64.90, 63.43, 61.68, 54.35, 54.13, 34.65, 34.29, 34.14 (d, J=5.0 Hz), 31.84, 31.13 (d, J=2.7 Hz), 29.50 (d, J=3.1 Hz), 29.19 (d, J=5.0 Hz), 27.22, 26.85, 26.34, 26.06, 25.30, 25.06, 24.81, 22.64, 22.43, 14.09, 13.95. LC-MS: m/z 803.20 (M+H)+C₄₈H₉₁N₅O₄ (802.29).

LipidB23-6: ¹H NMR (400 MHZ, Chloroform-d) δ 7.75 (s, 1H), 4.86 (p, J=6.2 Hz, 1H), 4.37 (t, J=6.2 Hz, 2H), 4.26-3.76 (m, 6H), 2.87 (t, J=6.2 Hz, 2H), 2.80-2.57 (m, 3H), 2.57-2.31 (m, 4H), 2.17-1.69 (m, 5H), 1.69-0.99 (m, 53H), 0.99-0.74 (m, 9H).¹³C NMR (101 MHZ, Chloroform-d) δ 173.58, 74.10, 65.43, 64.89, 54.35, 54.13, 54.09, 48.92, 46.50, 34.65, 34.28, 34.17, 34.11, 31.83, 31.14, 31.11, 29.50, 29.47, 29.24, 29.21, 29.18, 27.25, 27.13, 26.93, 26.88, 26.33, 26.06, 25.29, 25.09, 24.82, 22.64, 22.43, 14.09, 13.94, 11.08. LC-MS: m/z 830.85 (M+H)⁺C₅₀H₉₅N₅O₄ (830.34).

LipidB23-7: ¹H NMR (400 MHZ, Chloroform-d) δ 7.84 (s, 1H), 4.86 (p, J=6.3 Hz, 1H), 4.38 (d, J=7.5 Hz, 2H), 4.19-3.89 (m, 6H), 2.96-2.71 (m, 4H), 2.42 (dt, J=10.7, 4.9 Hz, 4H), 2.29 (q, J=7.6 Hz, 4H), 2.03-1.70 (m, 8H), 1.68-1.46 (m, 9H), 1.44-1.16 (m, 42H), 0.89 (dt, J=12.8, 6.7 Hz, 10H). ¹³C NMR (101 MHZ, Chloroform-d) δ 173.60, 74.10, 65.44, 64.90, 63.39, 54.35, 54.14, 54.02, 34.65, 34.29, 34.18, 34.11, 31.83, 31.13, 29.50, 29.47, 29.24, 29.20, 29.18, 27.24, 27.10, 26.91, 26.87, 26.32, 26.05, 25.28, 25.08, 24.82, 22.63, 22.42, 14.08, 13.94. LC-MS: m/z 828.90 (M+H)⁺C₅₀H₉₃N₅O₄ (828.33).

LipidB23-8: ¹H NMR (400 MHZ, Chloroform-d) δ 7.61 (s, 1H), 4.86 (p, J=6.3 Hz, 1H), 4.36 (t, J=6.3 Hz, 2H), 4.21-4.03 (m, 4H), 3.72 (s, 1H), 2.86 (t, J=6.3 Hz, 2H), 2.75-2.54 (m, 5H), 2.43 (dq, J=9.6, 3.7 Hz, 3H), 2.37 (s, 2H), 2.29 (q, J=7.7 Hz, 4H), 2.05-1.69 (m, 5H), 1.60 (qd, J=9.8, 8.7, 5.0 Hz, 4H), 1.50 (d, J=6.1 Hz, 4H), 1.42-1.17 (m, 39H), 0.89 (dt, J=11.6, 6.8 Hz, 9H). ¹³C NMR (101 MHZ, Chloroform-d) δ 173.57, 74.10, 65.43, 64.89, 63.40, 54.54, 54.37, 54.14, 54.10, 52.95, 52.12, 48.86, 45.45, 34.64, 34.28, 34.16, 34.11, 31.83, 31.13, 31.11, 29.50, 29.47, 29.23, 29.20, 29.17, 27.24, 27.09, 26.87, 26.32, 26.06, 25.28, 25.07, 24.81, 22.63, 22.42, 14.08, 13.94. LC-MS: m/z 857.95 (M+H)⁺C₅₁H₉₆N₆O₄ (857.37).

LipidB23-9: ¹H NMR (400 MHZ, Chloroform-d) δ 4.86 (p, J=6.3 Hz, 1H), 4.19-4.02 (m, 3H), 3.09-2.80 (m, 1H), 2.67-2.40 (m, 2H), 2.29 (q, J=7.8 Hz, 3H), 2.06-1.70 (m, 5H), 1.61 (p, J=8.6, 8.1 Hz, 3H), 1.50 (d, J=6.1 Hz, 4H), 1.44-1.04 (m, 35H), 0.95-0.76 (m, 10H). ¹³C NMR (101 MHz, Chloroform-d) & 173.60, 74.11, 65.55, 65.05, 64.91, 63.54, 54.19, 53.99, 34.71, 34.66, 34.36, 34.28, 34.12, 31.85, 31.24, 31.17, 31.15, 30.02, 29.68, 29.52, 29.48, 29.30, 29.22, 26.36, 26.33, 26.14, 26.07, 25.31, 25.12, 24.86, 22.65, 22.45, 14.13, 14.10, 13.99, 13.96. LC-MS: m/z 788.80 (M+H)⁺C₄₇H₈₉N₅O₄ (788.26).

LipidB23-10: ¹H NMR (400 MHZ, Chloroform-d) δ 7.37 (s, 1H), 4.86 (p, J=6.3 Hz, 1H), 4.34 (t, J=6.2 Hz, 2H), 4.22-3.98 (m, 4H), 2.85 (t, J=6.2 Hz, 2H), 2.69 (t, J=7.6 Hz, 2H), 2.42 (t, J=9.3 Hz, 4H), 2.01-1.57 (m, 13H), 1.55-1.45 (m, 5H), 1.40-1.18 (m, 45H), 0.97 (t, J=7.4 Hz, 3H), 0.93-0.82 (m, 10H). ¹³C NMR (101 MHZ, Chloroform-d) δ 173.60, 74.13, 65.46, 64.91, 63.41, 54.44, 54.26, 54.22, 48.74, 34.68, 34.29, 34.20, 34.15, 31.87, 31.17, 31.13, 29.54, 29.51, 29.26, 29.24, 29.21, 27.71, 27.28, 27.16, 26.92, 26.09, 25.32, 25.11, 24.85, 22.81, 22.67, 22.46, 14.11, 13.97, 13.80. LC-MS: m/z 787.95 (M+H)⁺C₄₈H₉₀N₄O₄ (787.27).

LipidB23-11: ¹H NMR (400 MHZ, Chloroform-d) δ 7.40 (s, 1H), 4.86 (p, J=6.3 Hz, 1H), 4.35 (t, J=6.2 Hz, 2H), 4.20-4.01 (m, 4H), 2.86 (d, J=6.3 Hz, 2H), 2.58 (d, J=6.7 Hz, 2H), 2.42 (tt, J=10.6, 5.0 Hz, 4H), 2.28 (q, J=7.5 Hz, 4H), 2.04-1.70 (m, 7H), 1.62 (q, J=7.5 Hz, 5H), 1.50 (d, J=6.1 Hz, 5H), 1.27 (d, J=8.5 Hz, 48H), 0.98-0.82 (m, 16H). ¹³C NMR (101 MHZ, Chloroform-d) δ 173.57, 74.10, 65.44, 64.89, 63.39, 54.38, 54.21, 54.16, 48.68, 34.66, 34.28, 34.17, 34.13, 31.85, 31.15, 31.11, 29.52, 29.49, 29.22, 29.18, 28.74, 27.25, 27.10, 26.89, 26.34, 26.07, 25.30, 25.09, 24.82, 22.65, 22.44, 22.31, 14.09, 13.95. LC-MS: m/z 801.90 (M+H)+C₄₉H₉₂N₄O₄ (801.30).

LipidB23-12: ¹H NMR (400 MHZ, Chloroform-d) δ 7.32 (s, 1H), 4.86 (p, J=6.3 Hz, 1H), 4.31 (t, J=6.3 Hz, 2H), 4.18-4.02 (m, 4H), 2.83 (t, J=6.3 Hz, 2H), 2.41 (td, J=7.5, 4.1 Hz, 4H), 2.29 (q, J=7.8 Hz, 4H), 2.04-1.71 (m, 6H), 1.67-1.56 (m, 5H), 1.50 (d, J=6.1 Hz, 4H), 1.43-1.14 (m, 43H), 0.98-0.76 (m, 13H). ¹³C NMR (101 MHZ, Chloroform-d) δ 173.60, 120.45, 74.12, 65.46, 64.91, 63.41, 54.44, 54.24, 54.21, 48.78, 34.68, 34.30, 34.20, 34.15, 31.87, 31.17, 31.13, 29.54, 29.51, 29.26, 29.24, 29.20, 27.28, 27.15, 26.93, 26.91, 26.36, 26.09, 25.32, 25.11, 24.85, 22.67, 22.46, 14.11, 13.97, 7.66, 6.70. LC-MS: m/z 785.85 (M+H)⁺C₄₈H₈₈N₄O₄ (785.26).

LipidB23-13: ¹H NMR (400 MHZ, Chloroform-d) δ 7.66 (s, 1H), 4.92-4.61 (m, 4H), 4.39 (t, J=6.4 Hz, 2H), 4.18-4.02 (m, 4H), 3.92 (ddd, J=11.4, 7.9, 3.0 Hz, 1H), 3.56 (dt, J=10.6, 4.7 Hz, 1H), 2.89 (t, J=6.3 Hz, 2H), 2.51-2.39 (m, 4H), 2.29 (q, J=7.6 Hz, 4H), 2.04-1.68 (m, 8H), 1.67-1.45 (m, 14H), 1.45-1.18 (m, 43H), 0.89 (dt, J=11.5, 6.8 Hz, 9H). ¹³C NMR (101 MHZ, Chloroform-d) & 173.55 (d, J=2.2 Hz), 98.04, 74.09, 65.43, 64.89, 63.39, 62.25, 60.51, 54.36, 54.14, 54.09, 34.64, 34.29, 34.14, 34.12, 31.84, 31.14, 31.12, 30.47, 29.51, 29.48, 29.21, 29.17, 27.23, 26.86, 26.78, 26.33, 26.07, 25.40, 25.29, 25.08, 24.79, 22.64, 22.43, 19.37, 14.09, 13.95. LC-MS: m/z 859.95 (M+H)⁺C₅₁H₉₄N₄O₆ (859.34).

LipidB23-14: ¹H NMR (400 MHZ, Chloroform-d) δ 7.71 (s, 1H), 5.71 (s, 1H), 4.86 (p, J=6.2 Hz, 1H), 4.38 (t, J=6.4 Hz, 2H), 4.23-4.00 (m, 4H), 3.65 (dp, J=23.0, 7.4 Hz, 4H), 2.88 (t, J=6.4 Hz, 2H), 2.50-2.38 (m, 4H), 2.29 (q, J=7.9 Hz, 4H), 2.05-1.69 (m, 6H), 1.65-1.57 (m, 5H), 1.50 (t, J=6.1 Hz, 4H), 1.43-1.17 (m, 50H), 0.89 (dt, J=11.5, 6.7 Hz, 9H). ¹³C NMR (101 MHZ, Chloroform-d) δ 173.57, 74.10, 65.44, 64.90, 63.40, 61.54, 54.37, 54.15, 54.08, 34.66, 34.28, 34.13, 31.85, 31.15, 31.11, 29.52, 29.49, 29.24, 29.22, 29.18, 27.24, 26.87, 26.34, 26.07, 25.30, 25.10, 24.81, 22.65, 22.44, 15.17, 14.10, 13.95. LC-MS: m/z 848.10 (M+H)⁺C₅₀H₉₄N₄O₆ (847.32).

LipidB23-15: ¹H NMR (400 MHZ, Chloroform-d) δ 7.92-7.29 (m, 6H), 4.86 (p, J=6.3 Hz, 1H), 4.43 (t, J=6.1 Hz, 2H), 4.15-4.00 (m, 4H), 2.91 (t, J=6.1 Hz, 2H), 2.45 (q, J=7.0 Hz, 4H), 2.25 (dt, J=9.8, 7.5 Hz, 4H), 2.02-1.69 (m, 6H), 1.63-1.47 (m, 10H), 1.44-1.19 (m, 47H), 0.89 (dt, J=10.6, 6.8 Hz, 9H). ¹³C NMR (101 MHZ, Chloroform-d) δ 128.80, 127.98, 125.65, 120.58, 74.10, 65.47, 64.92, 63.39, 54.42, 54.26, 54.21, 49.01, 34.65, 34.31, 34.15, 31.87, 31.17, 31.13, 29.54, 29.51, 29.27, 29.24, 29.19, 27.33, 27.13, 26.94, 26.91, 26.35, 26.08, 25.32, 25.09, 24.82, 22.67, 22.45, 14.11, 13.97. LC-MS: m/z 821.75 (M+H)⁺C₅₁H₈₈N₄O₄ (821.29).

### Example 4 Cytotoxicity test

Detection method: Ionizable lipids LipidA-1 to LipidA-14, LipidB22-1 to LipidB22-15, and LipidB23-1 to LipidB23-15 were weighed and each prepared into a 100 mM mother liquor using DMSO; and the mother liquor was diluted with serum-free DMEM and prepared into solutions at concentrations of 100 μM, 50 μM, 25 μM, 12.5 μM and 6.25 μM.

Hela cells (human cervical cancer cells) were cultured in high-glucose DMEM complete medium (10% fetal bovine serum, 1% penicillin-streptomycin) at 37° C. and 5% CO₂. When the cells were grown to a density of about 90% and entered the logarithmic phase, the cells were digested with trypsin and seeded in a 96-well plate at a density of 5×10³ cells/well and cultured under the conditions of 37° C. and 5% CO₂ for 24 hours. The diluted samples were added to the cells, 5 replicate wells were set for the blank control group and each sample concentration, and the culture was continued for 48 hours. After 48 hours, 10 μL of CCK-8 working solution was added to each well, and the cells were incubated in the incubator for 2 hours. The absorbance value at 450 nm was subsequently measured using a microplate reader. The blank control group was used as the 100% survival rate control, and the percentage of the absorbance value of other groups to the absorbance value of the blank control group was the cell survival rate of the groups, that is: cell survival rate (%)=(ODₒₜₕₑᵣ/OD_{blank})×100%.

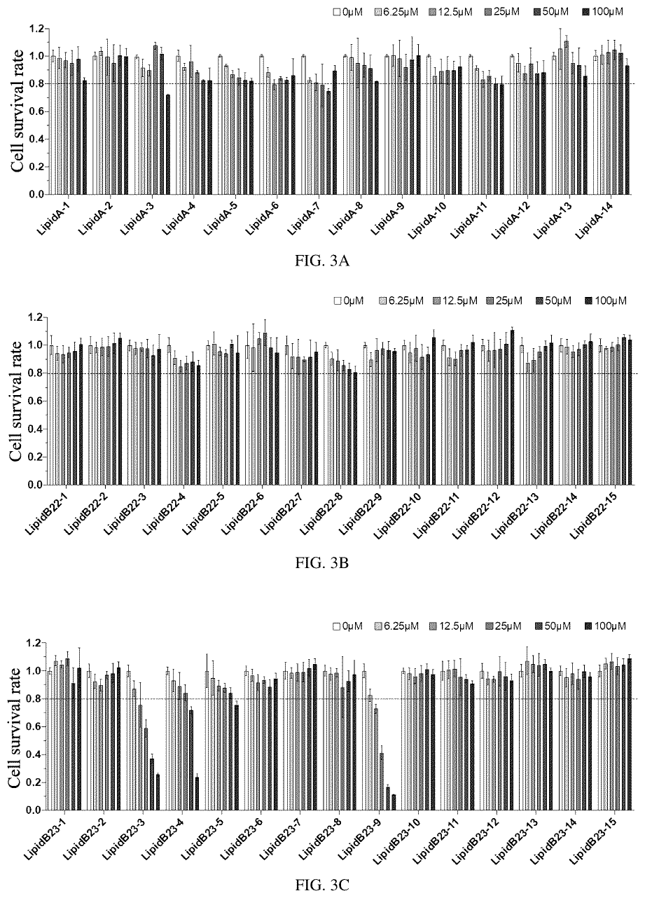

Results: It can be seen from FIG. 3 that the lipids with the saturated tail, LipidA-X series ionizable lipids and LipidB22-X series ionizable lipids, are generally less cytotoxic, and Hela cells still maintains the cell survival rate of more than 80% after incubation at a high concentration of 100 μM for 48 hours, which verifies the biosafety of these lipids and also indirectly reflects that the triazole linker does not bring additional toxicity and has a high biocompatibility.

### Example 5 Preparation of Lipid Nanoparticles (LNPs)

Preparation method: 20 mg of luciferase mRNA was diluted in 750 μL of citrate buffer (50 mM, pH=3.0), and the ionizable lipid was mixed with DSPC, CHO and DMG-PEG2000 in a molar ratio of 50:10:38.5:1.5 in 250 μL of absolute ethanol, wherein the amount of ionizable lipid to mRNA was ionizable lipid: mRNA=20:1 (m/m). The mRNA and lipid mixed solution were then thoroughly mixed and allowed to stand at room temperature for 20 min. LNPs were extruded using a LiposoFast-Basic LF-1 liposome preparation extruder. The liposome extruder used a polycarbonate membrane with a pore size of 100 nm and each sample was extruded 21 times. The LNP suspension was collected, placed in a dialysis bag (a molecular weight cutoff of 3500), and dialyzed in 1×PBS (pH=7.4) for 18 h. The LNP suspension after dialysis was recovered for further characterization and testing. The suspension was filtered through a 0.22 μm filter before adding to the cells. The prepared LNPs were named after ionizable lipids, such as LNPs prepared using SM-102 were named LSM-102, LNPs prepared using LipidA-1 were named LA-1, LNPs prepared using LipidB22-1 were named LB22-1, and so on.

### Example 6 Particle Size Analysis of Lipid Nanoparticles (LNPs)

Detection method: The particle size of LNPs was measured using a BrookHaven 90plus PALS dynamic light scattering instrument in 1×PBS (pH=7.4), and the LNP sample was added to about ⅔ of the height of the cuvette. Each sample was tested three times, and the average was taken.

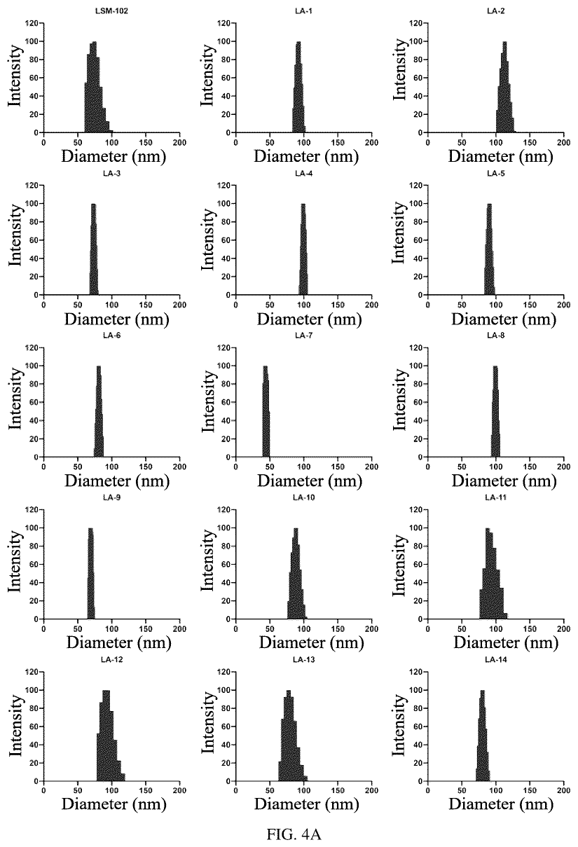

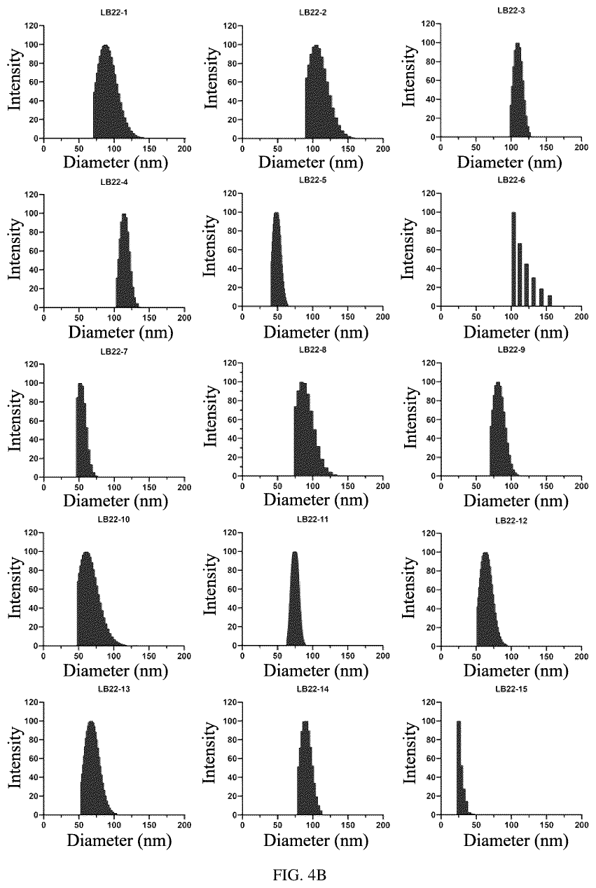

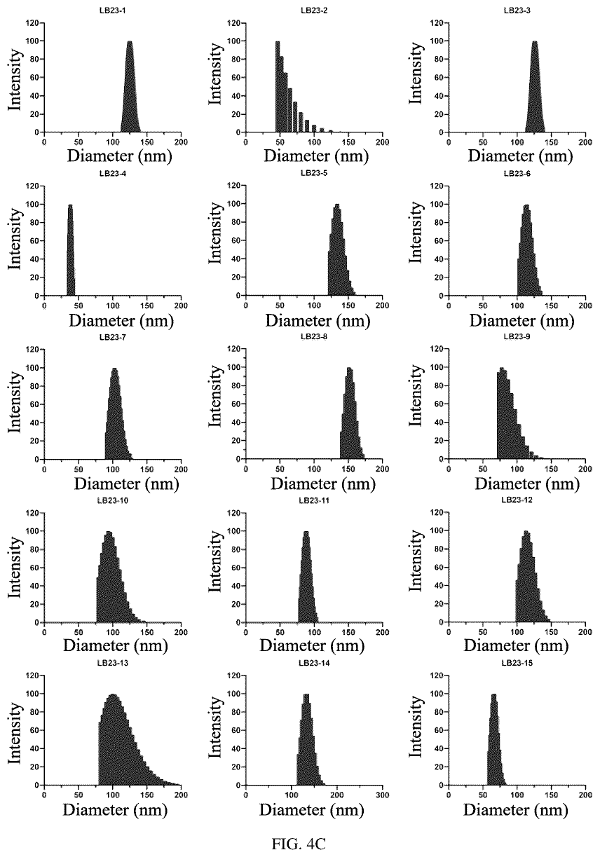

Results: The particle size distribution results of LNPs are shown in FIGS. 4A to 4C. The candidate lipids from the ionizable lipid A library and B library are successfully assembled into lipid nanoparticles, and the particle sizes of LA-X are about 100 nm with good uniformity (FIG. 4A); the particle sizes of LB22-X are mostly within 100 nm, and most of the candidate lipids have good uniformity (FIG. 4B); and the particle sizes of LB23-X are mostly distributed in the range of 100 nm to 150 nm, and most of the candidate lipids have good uniformity. Compared with LA-X and LB22-X, LB23-X has larger particle sizes (FIG. 4C).

### Example 7 Determination and Analysis of Zeta Potential of Lipid Nanoparticles (LNPs)

Zeta potential (ζ-potential) is a measure of the degree of mutual attraction or repulsion between nanoparticles and is an important indicator of colloidal characterization. The positive and negative values of the zeta potential represent the charge of the particles. Since the cell membrane is negatively charged, positively charged nanoparticles are theoretically easier to enter cells, which is beneficial for the delivery of mRNA.

Detection method: The zeta potential ((-potential) of LNPs was measured using a BrookHaven 90plus PALS dynamic light scattering instrument in water, and the LNP sample was added to about ⅔ of the height of the cuvette. Each sample was tested three times, and the average was taken.

Results: The zeta potential test results of LNPs are shown in Tables 4 to 6. The zeta potentials of all LNPs are positive, and thus the LNPs have the potential characteristics to successfully enter cells, supporting the expectation that LNPs can enter cells.

TABLE 4
ζ- potential of LNPs of A library and SM-102

| No. | LNPs | Zeta potential (mV) |
|---|---|---|
| 1 | LSM-102 | +15.77 |
| 2 | LA-1 | +16.68 |
| 3 | LA-2 | +4.90 |
| 4 | LA-3 | +10.64 |
| 5 | LA-4 | +14.63 |
| 6 | LA-5 | +15.28 |
| 7 | LA-6 | +13.45 |
| 8 | LA-7 | +11.79 |
| 9 | LA-8 | +18.71 |
| 10 | LA-9 | +25.41 |
| 11 | LA-10 | +19.26 |
| 12 | LA-11 | +11.46 |
| 13 | LA-12 | +25.04 |
| 14 | LA-13 | +8.64 |
| 15 | LA-14 | +13.45 |

TABLE 5
ζ-potential of LNPs of B library LB22-X

| No. | LNPs | Zeta potential (mV) |
|---|---|---|
| 1 | LB22-1 | +10.62 |
| 2 | LB22-2 | +1.08 |
| 3 | LB22-3 | +5.19 |
| 4 | LB22-4 | +7.32 |
| 5 | LB22-5 | +19.33 |
| 6 | LB22-6 | +20.61 |
| 7 | LB22-7 | +9.74 |
| 8 | LB22-8 | +12.68 |
| 9 | LB22-9 | +4.42 |
| 10 | LB22-10 | +13.99 |
| 11 | LB22-11 | +10.05 |
| 12 | LB22-12 | +0.82 |
| 13 | LB22-13 | +3.22 |
| 14 | LB22-14 | +15.37 |
| 15 | LB22-15 | +7.54 |

TABLE 6
ζ-potential of LNPs of B library LB23-X

| No. | LNPs | Zeta potential (mV) |
|---|---|---|
| 1 | LB23-1 | +4.05 |
| 2 | LB23-2 | +6.79 |
| 3 | LB23-3 | +9.20 |
| 4 | LB23-4 | +11.07 |
| 5 | LB23-5 | +16.66 |
| 6 | LB23-6 | +19.40 |
| 7 | LB23-7 | +17.37 |
| 8 | LB23-8 | +5.22 |
| 9 | LB23-9 | +12.81 |
| 10 | LB23-10 | +3.03 |
| 11 | LB23-11 | +4.99 |
| 12 | LB23-12 | +5.31 |
| 13 | LB23-13 | +19.58 |
| 14 | LB23-14 | +13.08 |
| 15 | LB23-15 | +16.29 |

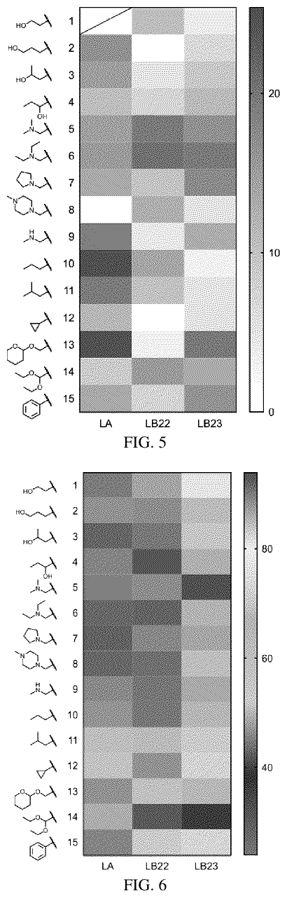

The zeta potentials were compared and analyzed with the structure of the ionizable lipids used. The results are shown in FIG. 5. There is no significant corresponding relationship between the zeta potential and the structure of the ionizable lipid used, because the zeta potentials of LNPs are the result of combined factors, which are not only influenced by the structure of ionizable lipids, but also by other factors.

### Example 8 Determination and Analysis of Entrapped Efficiency of Lipid Nanoparticles (LNPs)

Detection method: The entrapped efficiency of LNPs was measured using the Ribogreen fluorescent dye kit (Invitrogen). 50 μL of LNP sample was taken into a centrifuge tube and diluted to 350 μL by adding 1× TE buffer. 50 μL of the diluted LNP sample was added to a 96-well white plate, with 3 replicate wells for each sample. 2 μL (100:1 v/v) of Triton X-100 was added to the remaining 200 μL sample to lyse the LNPs, and the solution was mixed by vortexing and allowed to stand at room temperature for 10 min. After lysis, the solution was mixed by vortexing again and added to a 96-well white plate with 3 replicate wells for each sample. The standard curve was formulated using the RNA samples in the kit, and the standard concentrations were formulated at 4 μg/mL, 2 μg/mL, 1 μg/mL, 0.5 μg/mL, 0.25 μg/mL, 0.125 μg/mL and 0 μg/mL. Ribogreen fluorescent dye was diluted with 1× TE buffer at 1:200 (v/v), mixed and added to the samples in the well plate, 50 μL per well. After 2 to 5 minutes, the fluorescence signal value was measured using a TECAN Spark 10M multifunctional microplate reader, Ex/Em=480 nm/520 nm.

The fluorescence signal value of the sample without Triton X-100 lysis solution represents the free mRNA content, the fluorescence signal value of the sample with Triton X-100 lysis solution represents the total mRNA content, and the entrapped efficiency is the ratio of the total mRNA content minus the free mRNA content to the total mRNA content, that is:

$$Entrapped efficiency = \frac{Total mRNA content - Free MRNA content}{Total mRNA content} \times 100 \%$$

Results: The entrapped efficiency results of LNPs are shown in Tables 7 to 9, and these data indicate that mRNA is successfully entrapped in LNPs.

TABLE 7
Entrapped efficiency of LNPs of A library and SM-102

| No. | LNPs | Entrapped efficiency (%) |
|---|---|---|
| 1 | LSM-102 | 89.98 |
| 2 | LA-1 | 88.20 |
| 3 | LA-2 | 91.64 |
| 4 | LA-3 | 89.28 |
| 5 | LA-4 | 89.55 |
| 6 | LA-5 | 91.53 |
| 7 | LA-6 | 91.92 |
| 8 | LA-7 | 91.53 |
| 9 | LA-8 | 88.86 |
| 10 | LA-9 | 88.01 |
| 11 | LA-10 | 84.52 |
| 12 | LA-11 | 84.10 |
| 13 | LA-12 | 88.24 |
| 14 | LA-13 | 85.87 |
| 15 | LA-14 | 89.28 |

TABLE 8
Entrapped efficiency of LNPs of B library B22-X

| No. | LNPs | Entrapped efficiency (%) |
|---|---|---|
| 1 | LB22-1 | 86.68 |
| 2 | LB22-2 | 88.62 |
| 3 | LB22-3 | 90.29 |
| 4 | LB22-4 | 92.96 |
| 5 | LB22-5 | 88.67 |
| 6 | LB22-6 | 91.81 |
| 7 | LB22-7 | 89.07 |
| 8 | LB22-8 | 90.88 |
| 9 | LB22-9 | 90.40 |
| 10 | LB22-10 | 23.99 |
| 11 | LB22-11 | 63.05 |
| 12 | LB22-12 | 88.22 |
| 13 | LB22-13 | 84.19 |
| 14 | LB22-14 | 92.64 |
| 15 | LB22-15 | 83.39 |

TABLE 9
Entrapped efficiency of LNPs of B library B23-X

| No. | LNPs | Entrapped efficiency (%) |
|---|---|---|
| 1 | LB23-1 | 77.00 |
| 2 | LB23-2 | 62.37 |
| 3 | LB23-3 | 55.66 |
| 4 | LB23-4 | 49.35 |
| 5 | LB23-5 | 93.93 |
| 6 | LB23-6 | 85.47 |
| 7 | LB23-7 | 86.62 |
| 8 | LB23-8 | 84.52 |
| 9 | LB23-9 | 86.35 |
| 10 | LB23-10 | 65.24 |
| 11 | LB23-11 | 70.62 |
| 12 | LB23-12 | 72.26 |
| 13 | LB23-13 | 64.68 |
| 14 | LB23-14 | 39.09 |
| 15 | LB23-15 | 72.96 |

### Example 9 Evaluation of Delivery Efficiency of Lipid Nanoparticles (LNPs)

### Detection Method:

(1) Transfection: When the growth of Hela cells reached a density of about 90% and entered the logarithmic phase, the Hela cells were seeded into 96-well plates at a density of 15×10³ cells/well. After culturing for 24 hours, the concentration of mRNA entrapped in each of the LNP samples was calculated according to the entrapped efficiency, the amount of mRNA added to each well was 1 μg, and the volume of the LNP sample required to be added to the cells was calculated according to the entrapped mRNA concentration. The LNP sample was diluted with Opti-MEM medium, 100 μL was added to each well, and 5 replicate wells were set for each sample. The commercial transfection reagent Trans IT was used in the positive reference, and LNPs of SM-102 were used as the control group, naked mRNA was used in the negative control, and the concentration was the same as that of the replicate well.

(2) Luciferase assay: After transfection, the cells were cultured for 24 hours, the original culture medium was discarded, 100 μL of cell lysis buffer was added to each well, and the cells were shaken for lysis at room temperature for 10 minutes. The cells were pipetted and mixed uniformly with a pipette and then transferred to a 96-well white plate. 80 μL of cell lysis buffer and 20 μL of luciferase substrate were added to each well, and then the bioluminescent signal was measured using a TECAN Spark 10M multifunctional microplate reader. The fluorescence intensity data were normalized to the untreated group.

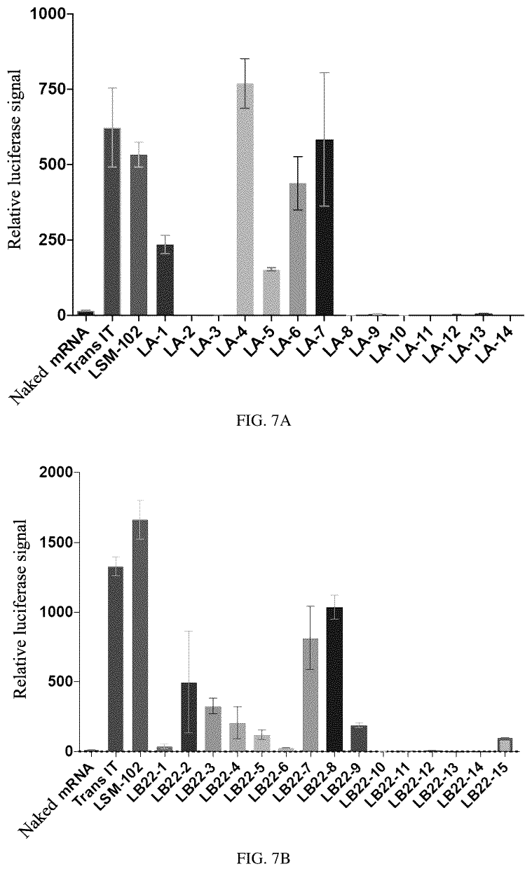

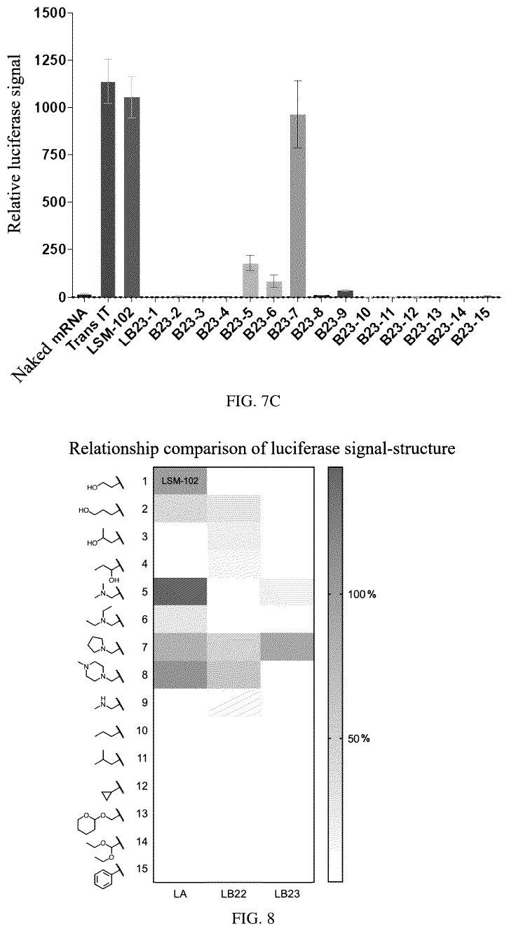

Results: The results of the efficiency measurement of LNPs delivering luciferase mRNA are shown in FIGS. 7A to 7C. Since naked mRNA is difficult to directly enter cells, directly adding naked mRNA into cells can hardly lead to luciferase expression. The LNP candidates LA-1, LA-4, LA-5, LA-6 and LA-7 in A library show significant luciferase expression, among which LA-7 has comparable effects to LSM-102 and has similar transfection efficiency to the commercial mRNA transfection reagent Trans IT; LA-4 is more effective than LSM-102 and Trans IT in producing a significant luciferase expression signal (FIG. 7A); the LNP candidates LB22-2, LB22-3, LB22-4, LB22-5, LB22-7, LB22-8 and LB22-9 in B library all have certain luciferase expression signals, among which the LNP with the strongest signal is LB22-8, with a signal intensity of 62.3% of that of LSM-102 (FIG. 7B); and among the LB23 series LNPs, only LB23-5, LB23-6 and LB23-7 have certain luciferase expression signals, among which the LNP with the strongest signal is LB23-7, with a signal intensity of 91.4% of that of LSM-102, which is comparable to that of LSM-102 (FIG. 7C).

The luciferase mRNA expression signal of LSM-102 was taken as 100%. After normalization, the efficiency of LNPs in delivering mRNA and the ionizable lipid structure were analyzed for structure-efficiency relationship. The green part represents the expression of luciferase, and the darker the color, the higher the expression efficiency of luciferase mRNA, which reflects the effectiveness of the represented LNPs.

The results are shown in FIG. 8. It can be seen that among all the existing ionizable lipids, the most effective candidate lipid is LipidA-4, a lipid with a dimethylamine head. The expression level of the LipidA-4-based lipid nanoparticle LA-4 is 144.1% of that of LSM-102, which is approximately 123.5% of that of the commercial transfection reagent Trans IT. In general, the green part is concentrated in the structure of 1 to 9, the color block in the range of 5 to 8 is the darkest, and all the ionizable lipids corresponding to the range of 5 to 8 are lipids containing tertiary amine heads.

### Example 10 Analysis of Factors Affecting the Delivery Efficiency of Lipid Nanoparticles (LNPs)

Based on the experimental results, a correlation analysis was performed on the entrapped efficiency, particle size, zeta potential and luciferase expression of LNPs. The Pearson correlation coefficient is a measure of the degree of linear correlation between variables and is generally represented by the letter r. The calculation formula of the correlation coefficient r is:

$$r ( X , Y ) = \frac{Cov ( X , Y )}{\sqrt{Var [ X ] Var [ Y ]}}$$

where Cov(X, Y) is the covariance of X and Y, Var[X] is the variance of X, and Var[Y] is the variance of Y. The correlation coefficient r is distributed in the range of [−1, 1]. The closer the absolute value of r is to 1, the closer the correlation between the data groups is, and the closer the absolute value of r is to 0, the lower the correlation is. The positive or negative values of r represent whether the data are positively correlated or negatively correlated.

The calculation results are shown in Table 10. There is a certain correlation between the luciferase expression level and the entrapped efficiency. Since the two are related to the structure of ionizable lipids that are the key components of LNPs, the entrapped efficiency reflects to some extent the efficacy of LNPs in complexing with mRNA, and affects the effect of LNPs in delivering mRNA, thus affecting the expression of luciferase mRNA in the cells.

TABLE 10
Correlation analysis of the properties of LNPs

| Comparative data | | r | Correlation |
|---|---|---|---|
| Luciferase | Entrapped | 0.381 | Moderate |
| expression | efficiency | | |
| | Particle size | −0.090 | No |
| | Zeta potential | 0.021 | No |

The luciferase mRNA expression signal of LSM-102 was taken as 100%, and the experimental group with a relative luciferase expression level of more than 50% was listed as an effective expression group.

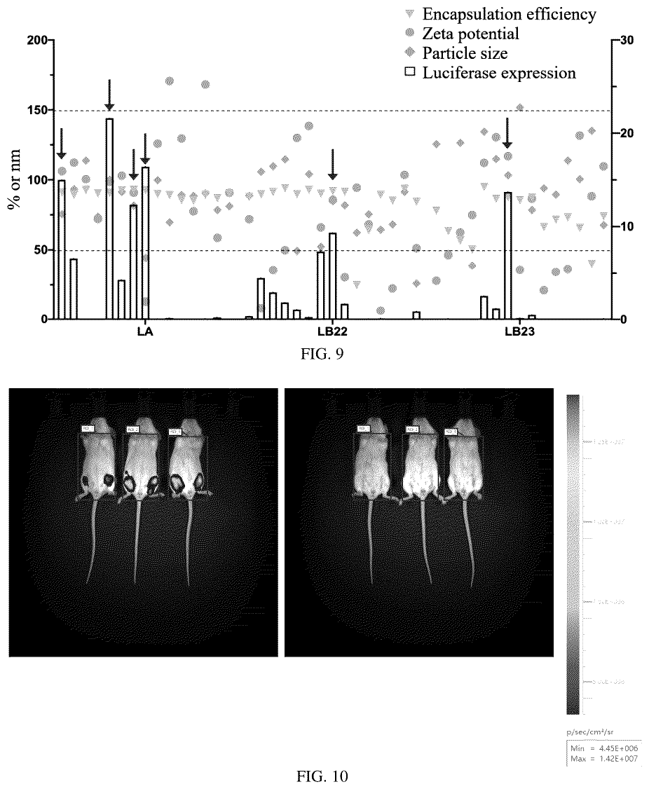

The results are shown in FIG. 9. The entrapped efficiency of LNP samples with significantly increased luciferase expression is around 90%.

### Example 11 Analysis of In Vivo Delivery Efficiency and Tissue Distribution of Lipid Nanoparticles (LNPs)

### Detection Method:

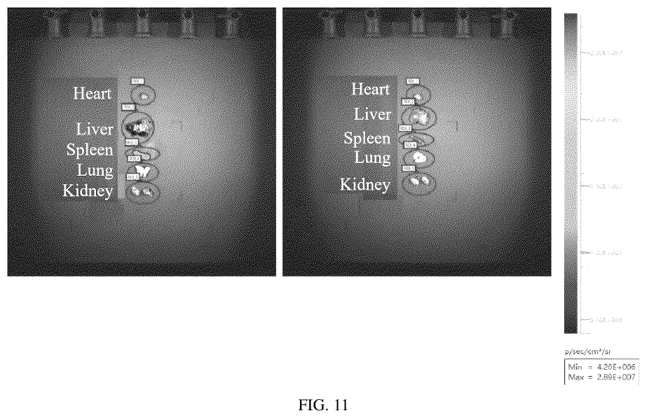

(1) Analysis of in vivo delivery efficiency in mice: Mice (BALB/C, Experimental Animal Center of Yangzhou University) were purchased and divided into two groups, with three mice in each group. One group of mice were administered by intramuscular injection (LNPs based on LB23-7 molecules, loaded with mRNA that can express Luciferase) in the left and right hind legs at a dose of 10 μg mRNA per mouse, and another group were administered with PBS by intramuscular injection in the left and right hind legs as a blank control. 6 h after administration, Luciferase signal imaging was performed on the whole body of the mouse using a small animal in vivo imaging device, and the expression efficiency of mRNA in vivo was determined according to the intensity of the bioluminescent signals. The results are as shown in FIG. 10. A strong chemiluminescence signal was detected 6 h after LNPs were injected into the muscle (left figure). It proves that LNPs based on LB23-7 molecules are efficiently expressed in the muscle.

(2) Analysis of tissue distribution in mice: Mice (BALB/C, Experimental Animal Center of Yangzhou University) were purchased and divided into two groups, with three mice in each group. One group of mice were administered by tail vein injection (LNPs based on LB23-7 molecules, loaded with mRNA that can express Luciferase) at a dose of 30 μg mRNA per mouse, and another group were administered with PBS by tail vein injection as a blank control. 24 h after administration, Luciferase signal imaging was performed on the mouse organs (heart, liver, spleen, lung and kidney) using a small animal in vivo imaging device. The expression of mRNA in various organs in the body was determined according to the intensity of the bioluminescent signals, thereby studying the distribution of LNPs in the body after administration via the tail vein. The results are shown in FIG. 11. After LNPs were administrated into the tail vein, a strong chemiluminescent signal was detected in the spleen of the mouse, the chemiluminescent signal in the liver was weak, and almost no chemiluminescent signals were detected in the other organs (left figure); and the imaging results of mice injected with PBS showed that no chemiluminescent signals were detected in various organs (right figure). It proves that LNPs are mainly distributed in the spleen region within 24 hours and enters the liver in a small quantity after entering the bodies of the mice through tail veins.

All features disclosed in this specification may be combined in any combination. Each feature disclosed in this specification may be replaced by an alternative feature serving the same, equivalent or similar purpose. Thus, unless expressly stated otherwise, each feature disclosed is only an example of a generic series of equivalent or similar features.

Based on the above descriptions, those skilled in the art can easily determine the essential characteristics of the present disclosure, and can make various changes and modifications to the present disclosure to adapt the present disclosure to various usages and conditions without departing from the spirit and scope of the present disclosure. Accordingly, other examples are within the scope of the appended claims.

1. A compound of formula (I), or a salt or an isomer thereof: 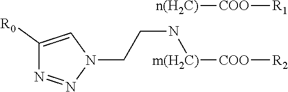
formula (I),
wherein R₀ is selected from C₁₋₄ alkyl, C₃₋₆ cycloalkyl, aryl or heteroaryl, the C₁₋₄ alkyl or C₃₋₆ cycloalkyl is optionally substituted with one or more —OH, —NR₀ₐR_{0b}, —NHR₀ₐ, —OR₀ₐ or 4-7 membered heterocyclyl containing 1-2 N, O or S atoms, and the aryl or heteroaryl is optionally substituted with C₁₋₃ alkyl, C₁₋₃ alkylalkoxy or halogen;
R₀ₐ and Rob are each independently selected from C₁₋₃ alkyl;
R₁ and R₂ are independently selected from C₂₋₂₀ alkyl or C₄₋₁₈ alkenyl; and
n and m are each independently selected from an integer of 1-9.

2. The compound according to claim 1, wherein n is 5, m is 7, R₁ is —(CH₂)₁₀CH₃, and R₂ is —CH((CH₂)₈CH₃)₂.

3. The compound according to claim 1, wherein both n and m are 7, and both R₁ and R₂ are —CH((CH₂)₈CH₃)₂.

4. The compound according to claim 1, wherein n is 5, m is 7, R₁ is —(CH₂)₃CH═CH(CH₂)₅CH₃, and R₂ is —CH((CH₂)₈CH₃)₂.

5. The compound according to claim 1, wherein R₀ is selected from —CH₂CH₃, —CH₂CH₂CH₃, —CH₂(CH₃)₂, —CH₂CH(CH₃)₂, —(CH₂)₃CH₃, —C(CH₃)₃, —CH(CH₃)CH₂CH₃, —CH₂CH₂OH, —CH(OH)CH₃, —CH₂CH₂CH₂OH, —CH₂CH(CH₃)OH, —CH(CH₃)CH₂OH, —C(OH)(CH₃)₂, —CH(OH)CH₂CH₃, —CH₂N(CH₂CH₃)₂, —CH₂N(CH₃)₂, —CH₂NHCH₃, —CH₂NHCH₂CH₃, —CH₂N(CH₃)CH₂CH₃, —CH(OCH₂CH₃)₂, 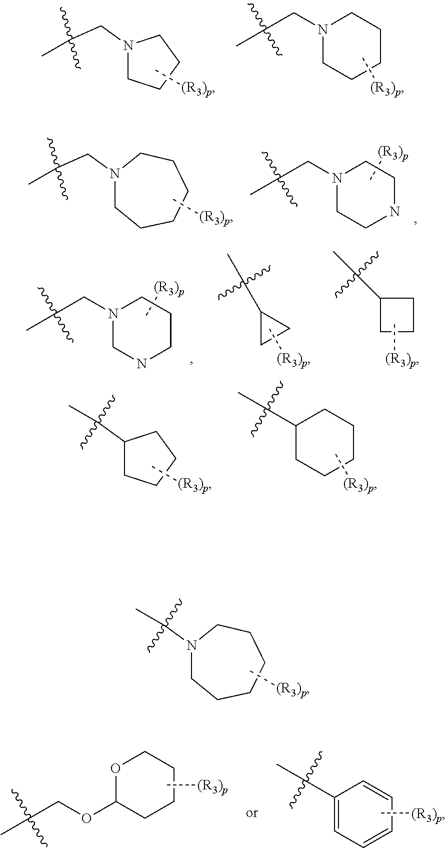
and R₃ is selected from C₁₋₃ alkyl, C₁₋₃ alkoxy or halogen, and p is selected from a natural number of 0-2.

6. The compound according to claim 1, wherein R₀ is selected from —CH₂CH₂CH₃, —CH₂CH(CH₃)₂, —CH₂CH₂OH, —CH₂CH₂CH₂OH, —CH₂CH(CH₃)OH, —CH(OH)CH₂CH₃, —CH₂N(CH₂CH₃)₂, —CH₂NHCH₃, —CH₂N(CH₃)₂, ═CH(OCH₂CH₃)₂,

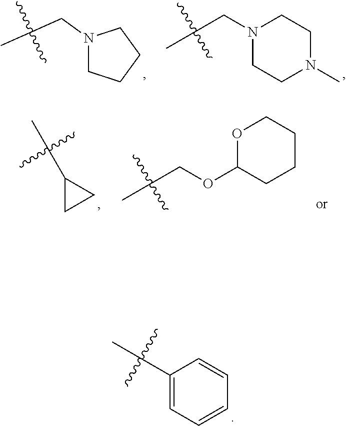

7. The compound according to claim 1, wherein R₁ is selected from C₈₋₂₀ alkyl or C₈₋₁₈ alkenyl.

8. The compound according to claim 1, wherein R₂ is selected from C₈₋₂₀ alkyl or C₈₋₁₈ alkenyl.

9. The compound according to claim 1, wherein R₁ is selected from —(CH₂)₇CH₃, —(CH₂): CH₃, —(CH₂)₉CH₃, —(CH₂)₁₀CH₃, —(CH₂)₁₁CH₃, —(CH₂)₁₂CH₃, —CH((CH₂)₄CH₃)₂, —CH((CH₂)₅CH₃)₂, —CH((CH₂)₆CH₃)₂, —CH((CH₂)₇CH₃)₂, CH((CH₂)₈CH₃)₂, —(CH₂)₂CH═CH(CH₂)₄CH₃, —(CH₂)₃CH═CH(CH₂)₃CH₃, —(CH₂)₄CH═CH(CH₂)₂CH₃, —(CH₂)₂CH═CH(CH₂)₃CH₃, —(CH₂)₃CH═CH(CH₂)₂CH₃, (CH₂)₂CH═CH(CH₂)₅CH₃, —(CH₂)₄CH═CH(CH₂)₃CH₃, —(CH₂)₅CH═CH(CH₂)₂CH₃, —(CH₂)₃CH═CH(CH₂)₄CH₃, —(CH₂)₄CH═CH(CH₂)₄CH₃, —(CH₂)₃CH═CH(CH₂)₅CH₃, (CH₂)₅CH═CH(CH₂)₃CH₃, —(CH₂)₂CH═CH(CH₂)₆CH₃, —(CH₂)₆CH═CH(CH₂)₂CH₃, —(CH₂)₄CH═CH(CH₂)₅CH₃, —(CH₂)₅CH═CH(CH₂)₄CH₃, —(CH₂)₃CH═CH(CH₂)₆CH₃, —(CH₂)₆CH═CH(CH₂)₃CH₃, —(CH₂)₂CH═CH(CH₂)₈CH₃ or —(CH₂)₈CH═CH(CH₂)₂CH₃.

10. The compound of formula (I) according to claim 1, wherein R₂ is selected from —(CH₂)₇CH₃, —(CH₂)₈CH₃, —(CH₂)₉CH₃, —(CH₂)₁₀CH₃, —(CH₂)₁₁CH₃, —(CH₂)₁₂CH₃, —CH((CH₂)₄CH₃)₂, —CH((CH₂)₅CH₃)₂, —CH((CH₂)₆CH₃)₂, —CH((CH₂)₇CH₃)₂, —CH((CH₂)₈CH₃)₂, (CH₂)₂CH═CH(CH₂)₄CH₃, (CH₂)₃CH═CH(CH₂)₃CH₃, (CH₂)₄CH═CH(CH₂)₂CH₃, —(CH₂)₂CH═CH(CH₂)₃CH₃, —(CH₂)₃CH═CH(CH₂)₂CH₃, —(CH₂)₂CH═CH(CH₂)₅CH₃, —(CH₂)₄CH═CH(CH₂)₃CH₃, —(CH₂)₅CH═CH(CH₂)₂CH₃, —(CH₂)₃CH═CH(CH₂)₄CH₃, —(CH₂)₄CH═CH(CH₂)₄CH₃, —(CH₂)₃CH═CH(CH₂)₅CH₃, —(CH₂)₅CH═CH(CH₂)₃CH₃, —(CH₂)₂CH═CH(CH₂)₆CH₃, —(CH₂)₆CH═CH(CH₂)₂CH₃, (CH₂)₄CH═CH(CH₂)₅CH₃, —(CH₂)₅CH═CH(CH₂)₄CH₃, —(CH₂)₃CH═CH(CH₂)₆CH₃, —(CH₂)₆CH═CH(CH₂)₃CH₃, —(CH₂)₂CH═CH(CH₂)₈CH₃ or —(CH₂): CH═CH(CH₂)₂CH₃.

11. The compound according to claim 1, wherein R₁ and R₂ are independently selected from —(CH₂)₇CH₃, —(CH₂)₈CH₃, —(CH₂)₉CH₃, —(CH₂)₁₀CH₃, —(CH₂)₁₁CH₃, —(CH₂)₁₂CH₃, —CH((CH₂)₄CH₃)₂, —CH((CH₂)₅CH₃)₂, —CH((CH₂)₆CH₃)₂, —CH((CH₂)₇CH₃)₂, —CH((CH₂)₈CH₃)₂, —(CH₂)₂CH═CH(CH₂)₄CH₃, —(CH₂)₃CH═CH(CH₂)₃CH₃, —(CH₂)₄CH═CH(CH₂)₂CH₃, —(CH₂)₂CH═CH(CH₂)₃CH₃, —(CH₂)₃CH═CH(CH₂)₂CH₃, —(CH₂)₂CH═CH(CH₂)₅CH₃, —(CH₂)₄CH═CH(CH₂)₃CH₃, —(CH₂)₅CH═CH(CH₂)₂CH₃, —(CH₂)₃CH═CH(CH₂)₄CH₃, —(CH₂)₄CH═CH(CH₂)₄CH₃, —(CH₂)₃CH═CH(CH₂)₅CH₃, —(CH₂)₅CH═CH(CH₂)₃CH₃, —(CH₂)₂CH═CH(CH₂)₆CH₃, —(CH₂)₆CH═CH(CH₂)₂CH₃, —(CH₂)₄CH═CH(CH₂)₅CH₃, —(CH₂)₅CH═CH(CH₂)₄CH₃, —(CH₂)₃CH═CH(CH₂)₆CH₃, —(CH₂)₆CH═CH(CH₂)₃CH₃, —(CH₂)₂CH═CH(CH₂)₈CH₃ or —(CH₂): CH═CH(CH₂)₂CH₃.

12. The compound according to claim 1, wherein the compound is selected from the following compounds LipidA-1 to LipidA-14, LipidB22-1 to LipidB22-15, LipidB23-1 to LipidB23-15, or salts or isomers thereof.

13. A delivery carrier, comprising the compound according to claim 1 and an accessory molecule.

14. The delivery carrier according to claim 13, wherein the accessory molecule comprises: a phospholipid, a structural lipid and a PEGylation lipid.

15. The delivery carrier according to claim 13, wherein the delivery carrier further comprises an active ingredient, and the active ingredient is selected from at least one of DNA, RNA, a protein or a pharmaceutically active molecule.

16. The delivery carrier according to claim 15, wherein the protein is selected from at least one of an antibody, an enzyme, a recombinant protein, a polypeptide or a short peptide, and the RNA is selected from at least one of mRNA, siRNA, aiRNA, miRNA, dsRNA, aRNA or lncRNA.

17. The delivery carrier according to claim 13, wherein the delivery carrier is a lipid nanoparticle.

18. A process comprising preparing lipid nanoparticles using the compound of formula (I) according to claim 1.
